(19) United States
(12) Patent Application Publication
(10) Pub. No.: US 2020/0181708 A1
(43) Pub. Date: Jun. 11, 2020

(54) MICRO-RNAS THAT MODULATE LYMPHANGIOGENESIS AND INFLAMMATORY PATHWAYS IN LYMPHATIC VESSEL CELLS

(71) Applicant: THE TEXAS A&M UNIVERSITY SYSTEM, COLLEGE STATION, TX (US)

(72) Inventors: MARIAPPAN MUTHUCHAMY, COLLEGE STATION, TX (US); SANJUKTA CHAKRABORTY, COLLEGE STATION, TX (US)

(21) Appl. No.: 16/796,576

(22) Filed: Feb. 20, 2020

Related U.S. Application Data
(63) Continuation of application No. 15/035,488, filed on May 10, 2016.
(60) Provisional application No. 61/903,602, filed on Nov. 13, 2013.

(51) Int. Cl. C12Q 1/6883; C12N 15/113; A61K 31/713

(52) U.S. Cl. CPC C12Q 1/6883; C12N 15/113; A61K 31/713; C12Q 2600/158; C12Q 2600/106; C12N 2310/141; C12N 2310/113; C12Q 2600/178; C12Q 2600/136; C12N 2320/30

(57) ABSTRACT

The subject invention pertains to methods of identifying miRNAs that are differentially expressed in a lymphatic vessel cell under a proinflammatory stimulus. The invention also pertains to profiles of miRNAs that are differentially expressed in a lymphatic vessel cell under a proinflammatory stimulus and their use as biomarkers for diagnosis of inflammation-mediated diseases. The current invention also provides therapeutic agents for the treatment of inflammation-mediated lymphatic diseases wherein the therapeutic agents are capable of modulating the activity of the miRNAs differentially expressed in a lymphatic vessel cell under a proinflammatory stimulus.

### CROSS-REFERENCE TO RELATED APPLICATIONS

This application is a continuation of U.S. application Ser. No. 15/035,488, filed May 10, 2016, which is the U.S. national stage application of International Patent Application No. PCT/US2014/065210, filed Nov. 12, 2014, which claims the benefit of U.S. Provisional Application Ser. No. 61/903,602, filed Nov. 13, 2013, the disclosures of which are hereby incorporated by reference in their entirety, including all figures, tables and amino acid or nucleic acid sequences.

This invention was made with government support under KO2-HL 086650 awarded by the National Institutes of Health. The government has certain rights in the invention.

The Sequence Listing for this application is labeled “Seq-List.txt” which was created on Nov. 12, 2014 and is 38 KB. The entire content of the sequence listing is incorporated herein by reference in its entirety.

### BACKGROUND OF THE INVENTION

The lymphatic system is a network of nodes and interconnected vessels, which plays a vital role in body fluid homeostasis, transport of dietary fat and cancer metastasis. Its involvement in immune cell trafficking and sensitivity to inflammatory mediators makes it a pivotal player in inflammation (von der Weid and Muthuchamy 2010; Zgraggen, Ochsenbein et al. 2013).

Lymphatic endothelial cells (LECs) at a site of inflammation have been shown to both actively participate in and regulate the inflammatory processes and host immune responses, thereby emerging as major players in both progression and resolution of the inflammatory state (Randolph, Angeli et al. 2005; Ji 2007; Pober and Sessa 2007; Podgrabinska, Kamalu et al. 2009; Huggenberger, Siddiqui et al. 2011; Vigl, Aebischer et al. 2011). Since inflammation has been shown to act as a primary trigger for pathological lymphangiogenesis, a number of proinflammatory cytokines have also been shown to function as pro-lymphangiogenic factors (Flister, Wilber et al. 2010; Kim, Kataru et al. 2012; Ran and Montgomery 2012). However, it is unclear whether lymphangiogenesis is beneficial or detrimental for the resolution of inflammation (Alexander, Chaitanya et al. 2010; Huggenberger, Siddiqui et al. 2011). Inflamed lymphatic endothelium has been shown to promote the exit of leukocytes, from tissue to afferent lymphatics through newly induced expression of the adhesion molecules stimulated by the proinflammatory cytokine, tumor necrosis factor-α (TNF-α) (Johnson, Clasper et al. 2006). TNF-α rapidly up-regulates ICAM-1, VCAM-1, and E-selectin in LECs, together with synthesis and release of several chemotactic agents, including the key inflammatory CC chemokines CCL5, CCL2, CCL20 and CCL21 (Johnson, Clasper et al. 2006; Sawa, Sugimoto et al. 2007; Sawa and Tsuruga 2008; Johnson and Jackson 2010). The role of TNF-α in regulating endothelial responses and tissue remodeling is typically characterized at the cellular level by rapid activation of the transcription factor, NF-κB and its downstream regulation of proinflammatory genes including cytokines, chemokines, and adhesion molecules (Lawrence 2009).

LECs have also been shown to express a number of toll-like receptors (TLRs) including TLR1-6 and TLR9, stimulation of which induces expression of the inflammatory cytokines IL-1β, TNF-α, and IL-6 (Pegu, Qin et al. 2008). Thus, LECs have emerged as an important source of inflammatory cytokines during pathogen-driven inflammation or in response to other inflammatory stimuli. LECs in turn respond to inflammatory cytokines by up-regulating chemokines, adhesion molecules, and other cytokines, indicating that LECs are also affected by the local inflammatory milieu present at sites of infection or vaccination (Pegu, Qin et al. 2008). Although a large number of these molecules expressed by inflamed LECs have been described (Sawa, Sugimoto et al. 2007; Sawa and Tsuruga 2008; Chaitanya, Franks et al. 2010), a critical group of potential regulators of the inflammatory mechanisms, namely the microRNAs (miRNAs), remain completely unexplored in the lymphatics. miRNAs are a recently recognized class of highly conserved, noncoding short RNA molecules that regulate gene expression at the post-transcriptional level (Kim 2005). They have been widely implicated in the regulation of endothelial dysfunction and pathologies, and have assumed a particularly significant role in regulation of inflammatory mechanisms (Suarez and Sessa 2009; Wu, Yang et al. 2009; O'Connell, Rao et al. 2012). The knockdown of a key miRNA-processing enzyme, DICER, has been shown to severely abrogate angiogenesis during mouse development, thereby underscoring the importance of miRNAs in vascular endothelial cell biology (Kuehbacher, Urbich et a. 2007; Suarez, Fernandez-Hernando et a. 2007).

Moreover, several miRNAs have been associated with regulation of endothelial cell migration, proliferation, regulation of nitric oxide production, tumor angiogenesis, wound healing, and vascular inflammation, and directly contribute to vascular pathologies (Urbich, Kuehbacher et al. 2008). Only two studies have investigated the role of miRNAs in the lymphatic vasculature in the context of development and lineage specification of LECs. It has been shown that miR-31 functions as a negative regulator of lymphatic development (Pedrioli, Karpanen et al. 2010). Kazenwadel et al., (Kazenwadel, Michael et al. 2010) have shown that Prospero Homeobox 1 (Prox1) expression is negatively regulated by miR-181 in LECs, providing important evidence of mechanisms underlying lymphatic vessel cell programming during development and neolymphangiogenesis.

The only miRNA shown to have a role in lymphangiogenesis is miR-1236, which targets VEGFR3 to inhibit inflammatory lymphangiogenesis (Jones, Li et al. 2012). Hence, it is clear that our understanding of miRNAs regulating gene networks involved in various lymphatic endothelial functions is very scant.

Lymphatic endothelial cells (LECs) and lymphatic muscle cells (LMCs) at a site of inflammation actively participate in both progression and resolution of inflammation. Clinical and preclinical studies indicate a relation between growth of new lymphatic vessels or lymphangiogenesis, or lymphatic dysfunction and inflammatory disorders.

While lymphangiogenesis is necessary to relieve the severity of acute skin inflammation and reduce dermal edema, by improving lymph flow, thereby decreasing edema, increased lymphangiogenesis promotes cancer metastasis and graft rejection. Hence any therapeutic modulation of inflammatory lymphangiogenesis and/or lymphatic inflammation needs to be designed and refined according to the context of the inflammation and purpose of intervention.

The current invention provides methods of identifying targets, such as miRNAs, in lymphatic vessel cells that are involved in lymphangiogenesis and/or lymphatic inflammation; miRNA targets and methods of using those targets to modulate lymphangiogenesis and/or lymphatic inflammation in the lymphatic system to treat inflammation-mediated lymphatic diseases; methods of diagnosing inflammation-mediated lymphatic diseases using the miRNA; methods of treating inflammation-mediated lymphatic diseases using the miRNA; and kits, for example, microarray chips, that can be used in the diagnosis of inflammation-mediated lymphatic diseases.

### BRIEF SUMMARY OF THE INVENTION

Various embodiments of the current invention provide methods of identifying miRNAs that are differentially expressed in a lymphatic vessel cell under a proinflammatory stimulus, the method comprising determining the miRNA expression profile of a first lymphatic vessel cell under a proinflammatory stimulus, determining the miRNA expression profile of a second lymphatic vessel cell in the absence of the proinflammatory stimulus, comparing the miRNA expression profile of the first lymphatic vessel cell with the miRNA expression profile of the second lymphatic vessel cell, and identifying the miRNAs that are differentially expressed in the first lymphatic vessel cell as compared to the second lymphatic vessel cell.

Certain embodiments of the current invention provide profiles of miRNAs that are differentially expressed in a lymphatic vessel cell under a proinflammatory stimulus as compared to a lymphatic vessel cell in the absence of the proinflammatory stimulus. The miRNAs belonging to the profiles of miRNAs differentially expressed in a lymphatic vessel cell under a proinflammatory stimulus can be used as biomarkers for the diagnosis of inflammation-mediated lymphatic diseases. Certain embodiments of the current invention provide microarrays of oligonucleotides corresponding to miRNAs that are differentially expressed in a lymphatic vessel cell under a proinflammatory stimulus.

Further embodiments of the current invention provide methods of treating an inflammation-mediated lymphatic disease, the method comprising administering to a subject in need thereof a pharmaceutically effective amount of an agent that can activate or inhibit an miRNA, wherein the miRNA belongs to a profile of miRNAs differentially expressed miRNA in a lymphatic vessel cell under a proinflammatory stimulus. The agent can be an oligonucleotide that can inhibit the miRNA, an oligonucleotide that can mimic the miRNA, or the miRNA.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. Schematic representation of specific miRNAs that are regulated by TNF-α treatment and some of their key functions in endothelial cells. The specific targets of the miRNAs regulating a particular physiological response are shown below each miRNA.

FIGS. 2A-2B. TNF-α mediated signaling in the lymphatic endothelium. A. Representative Western blot of protein samples from LECs treated with TNF-α (20 ng/ml) for 2 hr, 24 hr and 96 hr probed with phosphorylated and total forms of AKT, ERK1/2 and I-κB. Levels of PECAM1, β-Catenin, p-NF-κB, VE-CAD, N-CAD and ZEB1 were also measured. Experiments were carried out in triplicates and mean±SEM was calculated and the fold change values are given below for each blot. p<0.05 was considered as significant. B. Immunofluorescent image of activated NF-κB translocation in LECs. Magnification is 20×.

FIGS. 3A-3B. miR-9 inhibits NF-κB expression in LECs. A. Top panel shows the representative Western blots of samples from LECs transfected with different concentrations of miR-9 mimics or inhibitors as well as a control mi-sequence. β-Actin was used as a loading control. All the blots were done in triplicate and the ratio of NFκB and β-actin was calculated; mean±SEM was calculated and plotted. * p<0.05 compared with control was considered significant. Bottom panel shows immunofluorescent images of NF-κB expression in LECs transfected with 200 nM of miR-9 mimic or inhibitors. Magnification is 20×.

FIGS. 4A-4D. miR-9 promotes LEC tube formation and migration. LEC tube formation assay was performed as described in the Methods section under three different conditions: A. Complete media, B. Media supplemented with 3% serum and C. Media supplemented with 3% serum and TNF-α, for 16 hours. Cells were then visualized by staining with Calcein AM for 20 minutes and imaged with an inverted fluorescent microscope (Zeiss). Representative images from 3 independent experiments are shown in each panel. D. Branch points and tube lengths as indicated by arrowheads were quantified. **P<0.05 was considered significant, n=3.

FIGS. 5A-5C. Effects of miR-9 and TNF-α on VEGFR3 expression in lymphatics. A. miR-9 increases VEGFR3 expression in LECs. Top panel shows a representative Western blot of VEGFR3 in LECs transfected with 200 nM of miR-9 mimic or inhibitor or a control miR-sequence. β-Actin was used as a loading control. B. TNF-α treatment decreases VEGFR3 expression in LECs. Top panel shows a representative Western blot of VEGFR3 in LECs treated with TNF-α (20 ng/ml). Bottom panels (FIG. 5C): All the blots were done in triplicate and mean±SEM was calculated and plotted. * p<0.05 compared with control was considered significant.

FIGS. 6A-6B. miR-9 mediated signaling in LECs. A. Representative Western blots show expression of VE-Cadherin, eNOS and p-eNOS in LECs transfected with miR-9 mimics. The quantitative values obtained from 3 independent experiments are given at the bottom of each blot. B. miR-9 increases expression of β-catenin and eNOS in LECs. Immunofluorescent staining of eNOS and β-catenin in LECs transfected with miR-9 mimic or inhibitor or with a control miRNA. Magnification was 20×.

FIG. 7. Schematic representation of miR-9 as a modulator of lymphatic inflammation and lymphangiogenesis. In LECs, inflammatory stimuli as TNF-α induces the expression of miR-9. miR-9 regulates lymphatic inflammation by repressing NF-κB. On the other hand miR-9 expression inhibits the expression of VE-Cadherin, and activates β-Catenin and e-NOS in LECs. It also acts as a pro-lymphangiogenic molecule and induces the expression of VEGFR3, thereby promoting lymphangiogenesis. Pathways activated by miR-9 are also stipulated to have a role in endothelial to mesenchymal transition. Thus, activation of miR-9 provides a critical level of regulation in maintaining the balance between inflammation and lymphangiogenesis in inflamed LECs. Solid arrows indicate mechanisms that have been directly assessed in this study. Dashed arrows indicate previously published findings.

FIG. 8. miRNA 9 increases LEC proliferation. Role of miR 9 on LEC proliferation was determined using BrdU assay. HDLECs were transfected with miR 9 mimics, miR 9 inhibitor, TNF-α (100 ng/ml) or LPS (100 ng/ml) for 48 hrs. BrdU labeling solution was added to the cells and incubated for 2 hrs. BrdU incorporation was measured with the Cell Proliferation ELISA BrdU (chemiluminescence) kit (Roche Diagnostics, Indianapolis, Ind.) according to the manufacturer's protocol. The relative absorbance was measured on a spectrophotometer at 370 nm (with a reference wavelength 492 nm). The values are presented as mean±SEM, * p<0.05, n=3.

FIG. 9. miRNA 9 increases LEC viability. Role of miR 9 on LEC viability was determined using XTT assay based on the principle that XTT is only taken up by metabolically active cells. HDLECs were transfected with miR 9 mimics, miR 9 inhibitor, TNF-α (100 ng/ml) or LPS (100 ng/ml) for 48 hrs. XTT was added to the cells and incubated for 4 hrs. XTT assay was carried out according to the manufacturer's protocol (R&D Systems, Minneapolis, Minn.). The relative absorbance was measured on a spectrophotometer at absorbance at 490 nm (with a reference wavelength 630 nm). The values are presented as mean±SEM, * p<0.05, n=3.

FIG. 10. Expression of miR 9 is increased in vivo in mesenteric lymphatic vessels during inflammation. To determine the expression of miR 9 in vivo, lymphatic mesenteric vessels were isolated from control (PBS-treated) and LPS-treated rats (10 mg/kg body weight for 24 hrs). RNA was isolated and quantification of miR 9 levels compared to the housekeeping control RNU6 was carried out by real-time PCR. miR 9 shows a significant increase in lymphatic vessels from LPS-treated rats. The values are presented as mean±SEM, * p<0.05, n=3.

BRIEF DESCRIPTION OF THE SEQUENCES

| | SEQ ID | | SEQ ID | |
|---|---|---|---|---|
| miRNA | NO: | Pre-miRNA | NO: | Mature miRNA |
| hsa-miR- | 1 | UGGGGCCCUGGCUGGGAUAUCAUCAUAUAC | 76 | GGAUAUCAUCAUAUACUGU |
| 144-5p | | UGUAAGUUUGCGAUGAGACACUACAGUAUA | | AAG |
| | | GAUGAUGUACUAGUCCGGGCACCCCC | | |
| hsa-miR- | 2 | UGGGGCCCUGGCUGGGAUAUCAUCAUAUAC | 77 | UACAGUAUAGAUGAUGUAC |
| 144-3p | | UGUAAGUUUGCGAUGAGACACUACAGUAUA | | U |
| | | GAUGAUGUACUAGUCCGGGCACCCCC | | |
| hsa-miR- | 3 | GUAGCACUAAAGUGCUUAUAGUGCAGGUAG | 78 | UAAAGUGCUUAUAGUGCAG |
| 20a-5p | | UGUUUAGUUAUCUACUGCAUUAUGAGCACU | | GUAG |
| | | UAAAGUACUGC | | |
| hsa-miR- | 4 | GUAGCACUAAAGUGCUUAUAGUGCAGGUAG | 79 | ACUGCAUUAUGAGCACUUA |
| 20a-3p | | UGUUUAGUUAUCUACUGCAUUAUGAGCACU | | AAG |
| | | UAAAGUACUGC | | |
| hsa-miR- | 5 | GCCGGGAGGUUGAACAUCCUGCAUAGUGCU | 80 | UUGCAUAUGUAGGAUGUCC |
| 448 | | GCCAGGAAAUCCCUAUUUCAUAUAAGAGGG | | CAU |
| | | GGCUGGCUGGUUGCAUAUGUAGGAUGUCCC | | |
| | | AUCUCCCAGCCCACUUCGUCA | | |
| hsa-miR- | 6 | CCCUCGUCUUACCCAGCAGUGUUUGGGUGC | 81 | CGUCUUACCCAGCAGUGUU |
| 200c-5p | | GGUUGGGAGUCUCUAAUACUGCCGGGUAAU | | UGG |
| | | GAUGGAGG | | |
| hsa-miR- | 7 | CCCUCGUCUUACCCAGCAGUGUUUGGGUGC | 82 | UAAUACUGCCGGGUAAUGA |
| 200c-3p | | GGUUGGGAGUCUCUAAUACUGCCGGGUAAU | | UGGA |
| | | GAUGGAGG | | |
| hsa-miR- | 8 | GGCGCGUCGCCCCCCUCAGUCCACCAGAGC | 83 | CGGCUCUGGGUCUGUGGGG |
| 760 | | CCGGAUACCUCAGAAAUUCGGCUCUGGGUC | | A |
| | | UGUGGGGAGCGAAAUGCAAC | | |
| hsa-miR- | 9 | UGAGCCCUCGGAGGACUCCAUUUGUUUUGA | 84 | ACUCCAUUUGUUUUGAUGA |
| 136-5p | | UGAUGGAUUCUUAUGCUCCAUCAUCGUCUC | | UGGA |
| | | AAAUGAGUCUUCAGAGGGUUCU | | |
| hsa-miR- | 10 | UGAGCCCUCGGAGGACUCCAUUUGUUUUGA | 85 | CAUCAUCGUCUCAAAUGAG |
| 136-3p | | UGAUGGAUUCUUAUGCUCCAUCAUCGUCUC | | UCU |
| | | AAAUGAGUCUUCAGAGGGUUCU | | |
| hsa-miR- | 11 | CGGCCGGCCCUGGGUCCAUCUUCCAGUACA | 86 | CAUCUUCCAGUACAGUGUU |
| 141-5p | | GUGUUGGAUGGUCUAAUUGUGAAGCUCCUA | | GGA |
| | | ACACUGUCUGGUAAAGAUGGCUCCCGGGUG | | |
| | | GGUUC | | |
| hsa-miR- | 12 | CGGCCGGCCCUGGGUCCAUCUUCCAGUACA | 87 | UAACACUGUCUGGUAAAGA |
| 141-3p | | GUGUUGGAUGGUCUAAUUGUGAAGCUCCUA | | UGG |
| | | ACACUGUCUGGUAAAGAUGGCUCCCGGGUG | | |
| | | GGUUC | | |
| hsa-miR- | 13 | GUCAGAAUAAUGUCAAAGUGCUUACAGUGC | 88 | CAAAGUGCUUACAGUGCAG |
| 17-5p | | AGGUAGUGAUAUGUGCAUCUACUGCAGUGA | | GUAG |
| | | AGGCACUUGUAGCAUUAUGGUGAC | | |
| hsa-miR- | 14 | GUCAGAAUAAUGUCAAAGUGCUUACAGUGC | 89 | ACUGCAGUGAAGGCACUUG |
| 17-3p | | AGGUAGUGAUAUGUGCAUCUACUGCAGUGA | | UAG |
| | | AGGCACUUGUAGCAUUAUGGUGAC | | |
| hsa-miR- | 15 | GUGUUGGGGACUCGCGCGCUGGGUCCAGUG | 90 | GUGAAAUGUUUAGGACCAC |
| 203a | | GUUCUUAACAGUUCAACAGUUCUGUAGCGC | | UAG |
| | | AAUUGUGAAAUGUUUAGGACCACUAGACCC | | |
| | | GGCGGGCGCGGCGACAGCGA | | |
| hsa-miR- | 16 | GCGCCCGCCGGGUCUAGUGGUCCUAAACAU | 91 | UAGUGGUCCUAAACAUUUC |
| 203b-5p | | UUCACAAUUGCGCUACAGAACUGUUGAACU | | ACA |
| | | GUUAAGAACCACUGGACCCAGCGCGC | | |
| hsa-miR- | 17 | GCGCCCGCCGGGUCUAGUGGUCCUAAACAU | 92 | UUGAACUGUUAAGAACCAC |
| 203b-3p | | UUCACAAUUGCGCUACAGAACUGUUGAACU | | UGGA |
| | | GUUAAGAACCACUGGACCCAGCGCGC | | |
| hsa-miR- | 18 | AGUACCAAAGUGCUCAUAGUGCAGGUAGUU | 93 | CAAAGUGCUCAUAGUGCAG |
| 20b-5p | | UUGGCAUGACUCUACUGUAGUAUGGGCACU | | GUAG |
| | | UCCAGUACU | | |
| hsa-miR- | 19 | AGUACCAAAGUGCUCAUAGUGCAGGUAGUU | 94 | ACUGUAGUAUGGGCACUUC |
| 20b-3p | | UUGGCAUGACUCUACUGUAGUAUGGGCACU | | CAG |
| | | UCCAGUACU | | |
| hsa-miR- | 20 | UGUCGGGUAGCUUAUCAGACUGAUGUUGAC | 95 | UAGCUUAUCAGACUGAUGU |
| 21-5p | | UGUUGAAUCUCAUGGCAACACCAGUCGAUG | | UGA |
| | | GGCUGUCUGACA | | |
| hsa-miR- | 21 | UGUCGGGUAGCUUAUCAGACUGAUGUUGAC | 96 | CAACACCAGUCGAUGGGCU |
| 21-3p | | UGUUGAAUCUCAUGGCAACACCAGUCGAUG | | GU |
| | | GGCUGUCUGACA | | |
| hsa-miR- | 22 | AUACAGUGCUUGGUUCCUAGUAGGUGUCCA | 97 | CCUAGUAGGUGUCCAGUAA |
| 325-3p | | GUAAGUGUUUGUGACAUAAUUUGUUUAUUG | | GUGU |
| | | AGGACCUCCUAUCAAUCAAGCACUGUGCUA | | |
| | | GGCUCUGG | | |
| hsa-miR-9- | 23 | CGGGGUUGGUUGUUAUCUUUGGUUAUCUAG | 98 | UCUUUGGUUAUCUAGCUGU |
| 1-5p | | CUGUAUGAGUGGUGUGGAGUCUUCAUAAAG | | AUGA |
| | | CUAGAUAACCGAAAGUAAAAAUAACCCCA | | |
| hsa-miR-9- | 24 | CGGGGUUGGUUGUUAUCUUUGGUUAUCUAG | 99 | AUAAAGCUAGAUAACCGAA |
| 1-3p | | CUGUAUGAGUGGUGUGGAGUCUUCAUAAAG | | AGU |
| | | CUAGAUAACCGAAAGUAAAAAUAACCCCA | | |
| hsa-miR-9- | 25 | GGAAGCGAGUUGUUAUCUUUGGUUAUCUAG | 100 | UCUUUGGUUAUCUAGCUGU |
| 2-5p | | CUGUAUGAGUGUAUUGGUCUUCAUAAAGCU | | AUGA |
| | | AGAUAACCGAAAGUAAAAACUCCUUCA | | |
| hsa-miR-9- | 26 | GGAAGCGAGUUGUUAUCUUUGGUUAUCUAG | 101 | AUAAAGCUAGAUAACCGAA |
| 2-3p | | CUGUAUGAGUGUAUUGGUCUUCAUAAAGCU | | AGU |
| | | AGAUAACCGAAAGUAAAAACUCCUUCA | | |
| hsa-miR-9- | 27 | GGAGGCCCGUUUCUCUCUUUGGUUAUCUAG | 102 | UCUUUGGUUAUCUAGCUGU |
| 3-5p | | CUGUAUGAGUGCCACAGAGCCGUCAUAAAG | | AUGA |
| | | CUAGAUAACCGAAAGUAGAAAUGAUUCUCA | | |
| hsa-miR-9- | 28 | GGAGGCCCGUUUCUCUCUUUGGUUAUCUAG | 103 | AUAAAGCUAGAUAACCGAA |
| 3-3p | | CUGUAUGAGUGCCACAGAGCCGUCAUAAAG | | AGU |
| | | CUAGAUAACCGAAAGUAGAAAUGAUUCUCA | | |
| hsa-miR- | 29 | CUGAGGAGCAGGGCUUAGCUGCUUGUGAGC | 104 | AGGGCUUAGCUGCUUGUGA |
| 27a-5p | | AGGGUCCACACCAAGUCGUGUUCACAGUGG | | GCA |
| | | CUAAGUUCCGCCCCCCAG | | |
| hsa-miR- | 30 | CUGAGGAGCAGGGCUUAGCUGCUUGUGAGC | 105 | UUCACAGUGGCUAAGUUCC |
| 27a-3p | | AGGGUCCACACCAAGUCGUGUUCACAGUGG | | GC |
| | | CUAAGUUCCGCCCCCCAG | | |
| rno-miR- | 31 | CCUCGCUGACUCCGAAGGGAUGCAGCAGCA | 106 | CAGCAGCAAUUCAUGUUUU |
| 322-5p | | AUUCAUGUUUUGGAGUAUUGCCAAGGUUCA | | GGA |
| | | AAACAUGAAGCGCUGCAACACCCCUUCGUG | | |
| | | GGAAA | | |
| rno-miR- | 32 | CCUCGCUGACUCCGAAGGGAUGCAGCAGCA | 107 | AAACAUGAAGCGCUGCAAC |
| 322-3p | | AUUCAUGUUUUGGAGUAUUGCCAAGGUUCA | | A |
| | | AAACAUGAAGCGCUGCAACACCCCUUCGUG | | |
| | | GGAAA | | |
| rno-miR- | 33 | UGCAGUGCUUUAUCUAGUUGGCUGUCAGUC | 108 | GCAUGACACCAUACUGGGU |
| 878-5p | | ACGUGAAACUCAAGUGCAUGACACCAUACU | | AGA |
| | | GGGUAGAGGAGGGCUCA | | |
| hsa-miR- | 34 | GCAGUCCUCUGUUAGUUUUGCAUAGUUGCA | 109 | AGUUUUGCAUAGUUGCACU |
| 19a-5p | | CUACAAGAAGAAUGUAGUUGUGCAAAUCUA | | ACA |
| | | UGCAAAACUGAUGGUGGCCUGC | | |
| hsa-miR- | 35 | GCAGUCCUCUGUUAGUUUUGCAUAGUUGCA | 110 | UGUGCAAAUCUAUGCAAAA |
| 19a-3p | | CUACAAGAAGAAUGUAGUUGUGCAAAUCUA | | CUGA |
| | | UGCAAAACUGAUGGUGGCCUGC | | |
| rno-miR- | 36 | CCGGUGUAGUAGCCAUCAAAGUGGAGGCCC | 111 | CAUCAAAGUGGAGGCCCUC |
| 291a-5p | | UCUCUUGGGCCCGAGCUAGAAAGUGCUUCC | | UCU |
| | | ACUUUGUGUGCCACUGCAUGGG | | |
| rno-miR- | 37 | CCGGUGUAGUAGCCAUCAAAGUGGAGGCCC | 112 | AAAGUGCUUCCACUUUGUG |
| 291a-3p | | UCUCUUGGGCCCGAGCUAGAAAGUGCUUCC | | UGCC |
| | | ACUUUGUGUGCCACUGCAUGGG | | |
| rno-miR- | 38 | GUCUGAUGCCCUCAUCCUUGAGGGGCAUGA | 113 | CCUUGAGGGGCAUGAGGGU |
| 327 | | GGGUAGUCAGUAGCCUGAUGUCCCUCUUGA | | |
| | | UGGCACUUCGGACAUGUUGGAAUGGCUUGU | | |
| | | GAGG | | |
| hsa-miR- | 39 | UGGUACCUGAAAAGAAGUUGCCCAUGUUAU | 114 | GAAGUUGCCCAUGUUAUUU |
| 495-5p | | UUUCGCUUUAUAUGUGACGAAACAAACAUG | | UCG |
| | | GUGCACUUCUUUUUCGGUAUCA | | |
| hsa-miR- | 40 | UGGUACCUGAAAAGAAGUUGCCCAUGUUAU | 115 | AAACAAACAUGGUGCACUU |
| 495-3p | | UUUCGCUUUAUAUGUGACGAAACAAACAUG | | CUU |
| | | GUGCACUUCUUUUUCGGUAUCA | | |
| hsa-miR- | 41 | CACCUUGUCCUCACGGUCCAGUUUUCCCAG | 116 | GUCCAGUUUUCCCAGGAAU |
| 145-5p | | GAAUCCCUUAGAUGCUAAGAUGGGGAUUCC | | CCCU |
| | | UGGAAAUACUGUUCUUGAGGUCAUGGUU | | |
| hsa-miR- | 42 | CACCUUGUCCUCACGGUCCAGUUUUCCCAG | 117 | GGAUUCCUGGAAAUACUGU |
| 145-3p | | GAAUCCCUUAGAUGCUAAGAUGGGGAUUCC | | UCU |
| | | UGGAAAUACUGUUCUUGAGGUCAUGGUU | | |
| hsa-miR- | 43 | AAAGAUCCUCAGACAAUCCAUGUGCUUCUC | 118 | UCCUUCAUUCCACCGGAGU |
| 205-5p | | UUGUCCUUCAUUCCACCGGAGUCUGUCUCA | | CUG |
| | | UACCCAACCAGAUUUCAGUGGAGUGAAGUU | | |
| | | CAGGAGGCAUGGAGCUGACA | | |
| hsa-miR- | 44 | AAAGAUCCUCAGACAAUCCAUGUGCUUCUC | 119 | GAUUUCAGUGGAGUGAAGU |
| 205-3p | | UUGUCCUUCAUUCCACCGGAGUCUGUCUCA | | UC |
| | | UACCCAACCAGAUUUCAGUGGAGUGAAGUU | | |
| | | CAGGAGGCAUGGAGCUGACA | | |
| hsa-miR- | 45 | GGCCAGCUGUGAGUGUUUCUUUGGCAGUGU | 120 | UGGCAGUGUCUUAGCUGGU |
| 34a-5p | | CUUAGCUGGUUGUUGUGAGCAAUAGUAAGG | | UGU |
| | | AAGCAAUCAGCAAGUAUACUGCCCUAGAAG | | |
| | | UGCUGCACGUUGUGGGGCCC | | |
| hsa-miR- | 46 | GGCCAGCUGUGAGUGUUUCUUUGGCAGUGU | 121 | CAAUCAGCAAGUAUACUGC |
| 34a-3p | | CUUAGCUGGUUGUUGUGAGCAAUAGUAAGG | | CCU |
| | | AAGCAAUCAGCAAGUAUACUGCCCUAGAAG | | |
| | | UGCUGCACGUUGUGGGGCCC | | |
| hsa-miR- | 47 | CCACCCCGGUCCUGCUCCCGCCCCAGCAGC | 122 | CAGCAGCACACUGUGGUUU |
| 497-5p | | ACACUGUGGUUUGUACGGCACUGUGGCCAC | | GU |
| | | GUCCAAACCACACUGUGGUGUUAGAGCGAG | | |
| | | GGUGGGGGAGGCACCGCCGAGG | | |
| hsa-miR- | 48 | CCACCCCGGUCCUGCUCCCGCCCCAGCAGC | 123 | CAAACCACACUGUGGUGUU |
| 497-3p | | ACACUGUGGUUUGUACGGCACUGUGGCCAC | | AGA |
| | | GUCCAAACCACACUGUGGUGUUAGAGCGAG | | |
| | | GGUGGGGGAGGCACCGCCGAGG | | |
| hsa-miR- | 49 | AGUCUAGUUACUAGGCAGUGUAGUUAGCUG | 124 | AGGCAGUGUAGUUAGCUGA |
| 34c-5p | | AUUGCUAAUAGUACCAAUCACUAACCACAC | | UUGC |
| | | GGCCAGGUAAAAAGAUU | | |
| hsa-miR- | 50 | AGUCUAGUUACUAGGCAGUGUAGUUAGCUG | 125 | AAUCACUAACCACACGGCC |
| 34c-3p | | AUUGCUAAUAGUACCAAUCACUAACCACAC | | AGG |
| | | GGCCAGGUAAAAAGAUU | | |
| hsa-miR- | 51 | UGUUAAAUCAGGAAUUUUAAACAAUUCCUA | 126 | AUUCCUAGAAAUUGUUCAU |
| 384-5p | | GACAAUAUGUAUAAUGUUCAUAAGUCAUUC | | A |
| | | CUAGAAAUUGUUCAUAAUGCCUGUAACA | | |
| hsa-miR- | 52 | CACUGUUCUAUGGUUAGUUUUGCAGGUUUG | 127 | AGUUUUGCAGGUUUGCAUC |
| 19b-1-5p | | CAUCCAGCUGUGUGAUAUUCUGCUGUGCAA | | CAGC |
| | | AUCCAUGCAAAACUGACUGUGGUAGUG | | |
| hsa-miR- | 53 | CACUGUUCUAUGGUUAGUUUUGCAGGUUUG | 128 | UGUGCAAAUCCAUGCAAAA |
| 19b-1-3p | | CAUCCAGCUGUGUGAUAUUCUGCUGUGCAA | | CUGA |
| | | AUCCAUGCAAAACUGACUGUGGUAGUG | | |
| hsa-miR- | 54 | ACAUUGCUACUUACAAUUAGUUUUGCAGGU | 129 | AGUUUUGCAGGUUUGCAUU |
| 19b-1-5p | | UUGCAUUUCAGCGUAUAUAUGUAUAUGUGG | | UCA |
| | | CUGUGCAAAUCCAUGCAAAACUGAUUGUGA | | |
| | | UAAUGU | | |
| hsa-miR- | 55 | ACAUUGCUACUUACAAUUAGUUUUGCAGGU | 130 | UGUGCAAAUCCAUGCAAAA |
| 19b-1-3p | | UUGCAUUUCAGCGUAUAUAUGUAUAUGUGG | | CUGA |
| | | CUGUGCAAAUCCAUGCAAAACUGAUUGUGA | | |
| | | UAAUGU | | |
| hsa-miR- | 56 | UGAGUUUUGAGGUUGCUUCAGUGAACAUUC | 131 | AACAUUCAACGCUGUCGGU |
| 181-a-1-5p | | AACGCUGUCGGUGAGUUUGGAAUUAAAAUC | | GAGU |
| | | AAAACCAUCGACCGUUGAUUGUACCCUAUG | | |
| | | GCUAACCAUCAUCUACUCCA | | |
| hsa-miR- | 57 | UGAGUUUUGAGGUUGCUUCAGUGAACAUUC | 132 | ACCAUCGACCGUUGAUUGU |
| 181-a-1-3p | | AACGCUGUCGGUGAGUUUGGAAUUAAAAUC | | ACC |
| | | AAAACCAUCGACCGUUGAUUGUACCCUAUG | | |
| | | GCUAACCAUCAUCUACUCCA | | |
| hsa-miR- | 58 | AGAAGGGCUAUCAGGCCAGCCUUCAGAGGA | 133 | AACAUUCAACGCUGUCGGU |
| 181-a-2-5p | | CUCCAAGGAACAUUCAACGCUGUCGGUGAG | | GAGU |
| | | UUUGGGAUUUGAAAAAACCACUGACCGUUG | | |
| | | ACUGUACCUUGGGGUCCUUA | | |
| hsa-miR- | 59 | AGAAGGGCUAUCAGGCCAGCCUUCAGAGGA | 134 | ACCACUGACCGUUGACUGU |
| 181-a-2-3p | | CUCCAAGGAACAUUCAACGCUGUCGGUGAG | | ACC |
| | | UUUGGGAUUUGAAAAAACCACUGACCGUUG | | |
| | | ACUGUACCUUGGGGUCCUUA | | |
| hsa-miR- | 60 | CCUGUGCAGAGAUUAUUUUUUAAAAGGUCA | 135 | AACAUUCAUUGCUGUCGGU |
| 181-b-1-5p | | CAAUCAACAUUCAUUGCUGUCGGUGGGUUG | | GGGU |
| | | AACUGUGUGGACAAGCUCACUGAACAAUGA | | |
| | | AUGCAACUGUGGCCCCGCUU | | |
| hsa-miR- | 61 | CCUGUGCAGAGAUUAUUUUUUAAAAGGUCA | 136 | CUCACUGAACAAUGAAUGC |
| 181-b-1-3p | | CAAUCAACAUUCAUUGCUGUCGGUGGGUUG | | AA |
| | | AACUGUGUGGACAAGCUCACUGAACAAUGA | | |
| | | AUGCAACUGUGGCCCCGCUU | | |
| hsa-miR- | 62 | CUGAUGGCUGCACUCAACAUUCAUUGCUGU | 137 | AACAUUCAUUGCUGUCGGU |
| 181-b-2-5p | | CGGUGGGUUUGAGUCUGAAUCAACUCACUG | | GGGU |
| | | AUCAAUGAAUGCAAACUGCGGACCAAACA | | |
| hsa-miR- | 63 | CGGAAAAUUUGCCAAGGGUUUGGGGGAACA | 138 | AACAUUCAACCUGUCGGUG |
| 181-c-5p | | UUCAACCUGUCGGUGAGUUUGGGCAGCUCA | | AGU |
| | | GGCAAACCAUCGACCGUUGAGUGGACCCUG | | |
| | | AGGCCUGGAAUUGCCAUCCU | | |
| hsa-miR- | 64 | CGGAAAAUUUGCCAAGGGUUUGGGGGAACA | 139 | AACCAUCGACCGUUGAGUG |
| 181-c-3p | | UUCAACCUGUCGGUGAGUUUGGGCAGCUCA | | GAC |
| | | GGCAAACCAUCGACCGUUGAGUGGACCCUG | | |
| | | AGGCCUGGAAUUGCCAUCCU | | |
| hsa-miR- | 65 | GUCCCCUCCCCUAGGCCACAGCCGAGGUCA | 140 | AACAUUCAUUGUUGUCGGU |
| 181-d-5p | | CAAUCAACAUUCAUUGUUGUCGGUGGGUUG | | GGGU |
| | | UGAGGACUGAGGCCAGACCCACCGGGGGAU | | |
| | | GAAUGUCACUGUGGCUGGGCCAGACACGGC | | |
| | | UUAAGGGGAAUGGGGAC | | |
| hsa-miR- | 66 | GUCCCCUCCCCUAGGCCACAGCCGAGGUCA | 141 | CCACCGGGGGAUGAAUGUC |
| 181-d-3p | | CAAUCAACAUUCAUUGUUGUCGGUGGGUUG | | AC |
| | | UGAGGACUGAGGCCAGACCCACCGGGGGAU | | |
| | | GAAUGUCACUGUGGCUGGGCCAGACACGGC | | |
| | | UUAAGGGGAAUGGGGAC | | |
| hsa-miR- | 67 | UGAACAUCCAGGUCUGGGGCAUGAACCUGG | 142 | ACCUGGCAUACAAUGUAGA |
| 221-5p | | CAUACAAUGUAGAUUUCUGUGUUCGUUAGG | | UUU |
| | | CAACAGCUACAUUGUCUGCUGGGUUUCAGG | | |
| | | CUACCUGGAAACAUGUUCUC | | |
| hsa-miR- | 68 | UGAACAUCCAGGUCUGGGGCAUGAACCUGG | 143 | AGCUACAUUGUCUGCUGGG |
| 221-3p | | CAUACAAUGUAGAUUUCUGUGUUCGUUAGG | | UUUC |
| | | CAACAGCUACAUUGUCUGCUGGGUUUCAGG | | |
| | | CUACCUGGAAACAUGUUCUC | | |
| hsa-miR- | 69 | GCUGCUGGAAGGUGUAGGUACCCUCAAUGG | 144 | CUCAGUAGCCAGUGUAGAU |
| 221-5p | | CUCAGUAGCCAGUGUAGAUCCUGUCUUUCG | | CCU |
| | | UAAUCAGCAGCUACAUCUGGCUACUGGGUC | | |
| | | UCUGAUGGCAUCUUCUAGCU | | |
| hsa-miR- | 70 | GCUGCUGGAAGGUGUAGGUACCCUCAAUGG | 145 | AGCUACAUCUGGCUACUGG |
| 221-3p | | CUCAGUAGCCAGUGUAGAUCCUGUCUUUCG | | GU |
| | | UAAUCAGCAGCUACAUCUGGCUACUGGGUC | | |
| | | UCUGAUGGCAUCUUCUAGCU | | |
| hsa-miR- | 71 | CUGGGGGCUCCAAAGUGCUGUUCGUGCAGG | 146 | CAAAGUGCUGUUCGUGCAG |
| 93-5p | | UAGUGUGAUUACCCAACCUACUGCUGAGCU | | GUAG |
| | | AGCACUUCCCGAGCCCCCGG | | |
| hsa-miR- | 72 | CUGGGGGCUCCAAAGUGCUGUUCGUGCAGG | 147 | ACUGCUGAGCUAGCACUUC |
| 93-3p | | UAGUGUGAUUACCCAACCUACUGCUGAGCU | | CCG |
| | | AGCACUUCCCGAGCCCCCGG | | |
| hsa-miR- | 73 | UGCCCUGGCUCAGUUAUCACAGUGCUGAUG | 148 | CAGUUAUCACAGUGCUGAU |
| 101-1-5p | | CUGUCUAUUCUAAAGGUACAGUACUGUGAU | | GCU |
| | | AACUGAAGGAUGGCA | | |
| hsa-miR- | 74 | UGCCCUGGCUCAGUUAUCACAGUGCUGAUG | 149 | UACAGUACUGUGAUAACUG |
| 101-1-3p | | CUGUCUAUUCUAAAGGUACAGUACUGUGAU | | AA |
| | | AACUGAAGGAUGGCA | | |
| hsa-miR- | 75 | ACUGUCCUUUUUCGGUUAUCAUGGUACCGA | 150 | UACAGUACUGUGAUAACUG |
| 101-2-3p | | UGCUGUAUAUCUGAAAGGUACAGUACUGUG | | AA |
| | | AUAACUGAAGAAUGGUGGU | | |

### DETAILED DISCLOSURE OF THE INVENTION

The term “about” is used in this patent application to describe some quantitative aspects of the invention, for example, length of a polynucleotide in terms of the number of nucleotides or base pairs. It should be understood that absolute accuracy is not required with respect to those aspects for the invention to operate. When the term “about” is used to describe a quantitative aspect of the invention the relevant aspect may be varied by +10%. For example, a miRNA about 20 nucleotides long means a polynucleotide between 18 to 22 nucleotides long.

The current invention provides methods of identifying targets, such as miRNAs, in lymphatic vessel cells that are involved in lymphangiogenesis, lymphatic inflammation, and inflammation-mediated lymphatic diseases; miRNA targets and methods of using those targets to modulate lymphangiogenesis and/or lymphatic inflammation; methods of diagnosing inflammation-mediated lymphatic diseases using the miRNA; methods of treating inflammation-mediated lymphatic diseases using the miRNA; and kits, for example, microarray chips, that can be used in the diagnosis of inflammation-mediated lymphatic diseases.

Lymphatic inflammation is one of the underlying mechanisms of a range of pathological conditions including, but not limited to, airway inflammation, rheumatoid arthritis, inflammatory bowel disease (IBD), atherosclerosis, metabolic syndrome, cancer metastasis, psoriasis, organ transplantation, lymphedema, arthritis, and cardiovascular diseases. However the exact mechanism of regulation and prevention of inflammation-mediated lymphatic diseases is not well understood and there are no pharmacological therapies for lymphatic pathologies.

An miRNA is a small non-coding RNA molecule of about 20-25 nucleotides found in plants and animals. An miRNA functions in transcriptional and post-transcriptional regulation of gene expression. Encoded by eukaryotic nuclear DNA, miRNA functions via base-pairing with complementary sequences within mRNA molecules, usually resulting in gene silencing via translational repression or target degradation. microRNAs are transcribed by RNA polymerase II as large RNA precursors called pri-miRNAs. The pri-miRNAs are processed further in the nucleus to produce pre-miRNAs. Pre-miRNAs are about 70 nucleotides in length and are folded into imperfect stem-loop structures. The pre-miRNAs are then exported into the cytoplasm and undergo additional processing to generate miRNA. An miRNA profile of a cell or a tissue indicates expression levels of various miRNAs in the cell or the tissue.

A differentially expressed miRNA is the miRNA which is either over-expressed/up-regulated or under-expressed/down-regulated in a sample cell compared to a control cell. An miRNA is identified as a “differentially expressed miRNA” if the miRNA is expressed in the sample cell at least about 1.8 fold higher or lower than the corresponding miRNA in the control cell or has statistical significance (p value) of less than 0.05 when compared to the corresponding miRNA expression in the control cell.

A profile of differentially expressed miRNAs represents a set of miRNAs that are differentially expressed in a test/sample cell or tissue compared to a control/reference cell or tissue. The profile of differentially expressed miRNAs comprises a profile of down-regulated/under-expressed miRNAs and a profile of up-regulated/over-expressed miRNAs.

A proinflammatory stimulus is a stimulus capable of inducing inflammation in a cell. Non-limiting examples of proinflammatory stimulus include inflammatory cytokines, allergens, antigens, and lymphocyte-mediated inflammation.

A profile of differentially expressed miRNAs in a lymphatic vessel cell in response to a proinflammatory stimulus represents a set of miRNAs that are differentially expressed in a lymphatic vessel cell compared to a lymphatic vessel cell in the absence of the proinflammatory stimulus. The differential expression of an miRNA can occur in about 2 hours, about 4 hours, about 16 hours, about 24 hours, about 48 hours, about 72 hours, or about 96 hours after exposure to a proinflammatory stimulus.

For the purposes of this invention, a small molecule compound is a compound having a molecular weight of less than about 1000 daltons.

An antagomir of an miRNA or an miRNA antagomir is a polynucleotide capable of hybridizing with pri-miRNA, pre-miRNA, or mature miRNA via a sequence which is complementary or substantially complementary to the sequence of the miRNA. Typically, a sequence which is about 70%, about 75%, about 80%, about 85%, about 90%, about 95%, or about 100% complementary to target sequence is capable of hybridizing with the target sequence.

Mimics of an miRNA or miRNA mimics are small, double-stranded RNAs that mimic an endogenous miRNA and up-regulate the miRNA activity. miRNA mimics can be chemically modified RNAs to increase the stability, half-life, and/or bioavailability of the miRNAs. Non-limiting examples of chemical modifications of miRNAs include phosphodiester modification, phosphorothionate modification, ribose 2′-OH modification (for example, 2′O-methyl, 2′-fluoro, or 2′-methoxyethyl modification), ribose sugar modification (for example, Unlocked Nucleic acid (UNA)), modification of the nucleotide bases (for example, 5-bromo-, 5-iodo-, 2-thio-, 4-thio, dihydro, and pseudo-uracil), adenylation at the 3′ end, and locked nucleic acid modification. Additional non-limiting examples of modifications that can be performed on the agonists, antagomirs, or miRNA mimics of the current invention are provided by Bramsen et al. in “Chemical Modification of Small Interfering RNA”, Methods in Molecular Biology, Volume 721, pp. 77-103 (2011).

The aforementioned modifications can be used alone or in combination with each other. For example, a phosphate modification can be combined with a ribose sugar modification in the same miRNA. Additional examples of nucleotide modifications that increase stability, half-life, and/or bioavailability of the miRNAs are well-known to a person of ordinary skill in the art and such modifications are within the purview of the current invention.

Various embodiments of the current invention provide a method of identifying miRNAs that are differentially expressed in a lymphatic vessel cell under a proinflammatory stimulus, the method comprising:

a) culturing a first lymphatic vessel cell in the presence of the proinflammatory stimulus,

b) culturing a second lymphatic vessel cell in the absence of the proinflammatory stimulus,

c) isolating the miRNAs from the first lymphatic vessel cell and the second lymphatic vessel cell,

d) determining the expression profile of the miRNAs in the first lymphatic vessel cell and the second lymphatic vessel cell,

e) comparing the expression profile of miRNAs in the first lymphatic vessel cell with the expression profile of miRNAs in the second lymphatic vessel cell, and

f) identifying the miRNAs that are differentially expressed in the first lymphatic vessel cell when compared to the second lymphatic vessel cell.

An example of the technique to determining the miRNA expression profile in a cell is an miRNA microarray assay. Additional techniques of determining miRNA expression profiles, for example, PCR based techniques, are well-known to a person of ordinary skill in the art and such techniques are within the purview of this invention.

It is to be understood that diagnosis or the detection of the differential expression of the miRNA identified in lymphatic vessel cells under a proinflammatory stimulus with or associated with lymphatic inflammatory diseases. In the context of the present invention, these methods may be carried out by determining the amount of an miRNA molecule or a precursor molecule thereof by any method deemed appropriate. For example, the amount of an miRNA or a precursor molecule thereof may be determined by using a probe oligonucleotide that specifically detects the miRNA or precursor molecule to be analyzed or an amplification product of said miRNA or precursor.

The determination of the amount of an miRNA or a precursor molecule thereof, preferably by specific probe oligonucleotides, comprises the step of hybridizing an miRNA or a precursor molecule thereof or an amplification product thereof with a probe oligonucleotide that specifically binds to the transcript or the amplification product thereof. A probe oligonucleotide in the context of the present invention, is preferably a single-stranded nucleic acid molecule that is specific for said miRNA or a precursor molecule thereof and, preferably, comprises a stretch of nucleotides that specifically hybridizes with the target and, thus, is complementary to the target polynucleotide. Said stretch of nucleotides is, preferably, 85%, 90%, 95%, 99% or more preferably 100% identical to a sequence region comprised by a target polynucleotide (i.e., the miRNA disclosed herein). The degree of identity (percentage, %) between two or more nucleic acid sequences is, preferably, determined by the algorithms of Needleman and Wunsch or Smith and Waterman. To carry out the sequence alignments, the program PileUp (J. Mol. Evolution, 25, 351-360, 1987, Higgins 1989, CABIOS, 5: 151-153) or the programs Gap and BestFit (Needleman 1970, J. Mol. Biol. 48; 443-453 and Smith 1981, Adv. Appl. Math. 2; 482-489), which are part of the GCG software packet (Genetics Computer Group, 575 Science Drive, Madison, Wis., USA 53711, vers. 1991), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

The probe oligonucleotide may be labeled or contain other modifications including enzymes which allow a determination of the amount of an miRNA (quantification of the amount of miRNA) or precursor molecule thereof. Labeling can be done by various techniques well-known in the art depending on the label to be used.

The term “amount” as used herein encompasses the absolute amount of an miRNA or a precursor molecule (or an amplification product thereof), the relative amount or concentration thereof as well as any value or parameter which correlates thereto. Such values or parameters comprise intensity signal values from all specific physical or chemical properties obtained therefrom by direct measurements, e.g., intensity values or indirect measurements, e.g., expression levels determined from biological readout systems.

The term “comparing” as used herein encompasses comparing the amount of the miRNA or the precursor molecule thereof comprised by the sample to be analyzed (or of an amplification product of said miRNA or precursor molecule) with an amount of a suitable reference source. It is to be understood that comparing as used herein refers to a comparison of corresponding parameters or values, e.g., an absolute amount is compared to an absolute reference amount while a concentration is compared to a reference concentration, or an intensity signal obtained from a test sample is compared to the same type of intensity signal of a reference sample. The comparison referred to in step (b) of the method of the present invention may be carried out manually or may be, preferably, computer-assisted. For a computer-assisted comparison, the value of the determined amount may be compared to values corresponding to suitable references, which are stored in a database by a computer program. The computer program may further evaluate the result of the comparison, i.e., automatically provide the desired assessment in a suitable output format. Based on the comparison of the amount determined in step a) and the reference amount, it is possible to identify lymphatic inflammation in a sample from a subject. The terms “reference amount” or “reference sample(s)” refer to an amount of miRNA found in a biological sample obtained from one or more subjects not having lymphatic inflammation.

Comparing the expression profiles of miRNAs from two or more different cells can be performed using computer-assisted methods, for example, bioinformatics-based methods. Manual methods can also be used for comparing the expression profiles of miRNAs from two or more cells. Additional methods of comparing the expression profiles of miRNAs from two or more cells are well-known to a person of ordinary skill in the art and such methods are within the purview of this invention.

For example, the present invention provides devices adapted to carry out the various methods disclosed herein. For example, one aspect of the invention provides a device adapted for detecting the differential expression of the disclosed miRNA, or precursors thereof, that comprises: a) an analyzing unit comprising a detection agent for identifying up-regulated/over-expression of selected from one or more of miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p, and miR-19b, and down-regulated/under-expression of miRNAs selected from one or more of miR-101, miR-144, miR-20a, miR448, miR-760-5p, miR-136, miR-141, miR-291a-3p, miR-327, miR-495, miR-136, miR-144, miR-145, and miR-205, or of a precursor molecules thereof, wherein said analyzing unit is adapted for determining the amount(s) of at said at least one miRNA molecule or a precursor molecule thereof in a sample, and b) an evaluation unit comprising a computer comprising a tangibly embedded computer program code for carrying out a comparison of the determined amount(s) obtained from the analyzing unit with a reference amount (or reference amounts).

The term “device” as used herein relates to a computer system for automatically determining the amount of the disclosed miRNA within a sample and a reference sample. The data obtained by the computer system can be processed by, e.g., a computer program in order to diagnose or distinguish between the diseases/conditions disclosed herein and, in some cases, is a single device. The device may, accordingly, include an analyzing unit for the measurement of the amount of the miRNA in a sample and a computer unit for processing the resulting data for the quantification of the amounts of miRNA found in a sample and/or reference sample diagnosis.

In certain embodiments of the invention, the proinflammatory stimulus is mediated by a proinflammatory cytokine. Non-limiting examples of proinflammatory cytokines include interferons such as INF-γ; tumor necrosis factors such as TNF-α; interleukins such as IL-1, IL-2, IL-8, or IL-6; lipopolysaccharides; and neurogenic substance-p. Additional examples of proinflammatory cytokines are well-known to a person of ordinary skill in the art and such cytokines are within the purview of this invention.

Certain embodiments of the current invention provide profiles of differentially expressed miRNAs in a lymphatic vessel cell in the presence of a proinflammatory stimulus as compared to a lymphatic vessel cell in the absence of the proinflammatory stimulus. For example, a profile of differentially expressed miRNAs in a lymphatic vessel cell under a proinflammatory stimulus comprises one or more of miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p & miR-19b, miR-101, miR-144, miR-20a, miR448, miR-760-5p, miR-136, miR-141, miR-291a-3p, miR-327, miR-495, miR-136, miR-144, miR-145, and miR-205.

Various embodiments provide for a profile of differentially expressed miRNAs in a lymphatic vessel cell under a proinflammatory stimulus that comprises a profile of up-regulated/over-expressed miRNAs, the profile of over-expressed miRNAs comprising one or more of miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p, and miR-19b, and a profile of down-regulated/under-expressed miRNAs, the profile of down-regulated miRNAs comprises of one or more of miR-101, miR-144, miR-20a, miR448, miR-760-5p, miR-136, miR-141, miR-291a-3p, miR-327, miR-495, miR-136, miR-144, miR-145, and miR-205.

Additional embodiments of the current invention provide microarray chips consisting essentially of oligonucleotides corresponding to miRNAs belonging to a profile of differentially expressed miRNAs in a lymphatic vessel cell under a proinflammatory stimulus. For the purposes of this invention, a microarray chip “consisting essentially of oligonucleotides corresponding to miRNAs belonging to a profile of differentially expressed miRNAs in a lymphatic vessel cell under a proinflammatory stimulus indicates that the microarray chip contains only those miRNAs that are differentially expressed in a lymphatic vessel cell under a proinflammatory stimulus and does not contain miRNA whose expression remains unchanged in a lymphatic vessel cell under a proinflammatory stimulus. For example, the microarray chip of the current invention does not contain oligonucleotide probes corresponding to one or more (i.e., any combination) of the following miRNAs: let-7b, let-7c, let-7d, let-7e, let-7f, let-7i, miR-23a-3p, miR-23b-3p, miR-26a-5p, miR-26b-5p, miR-29a-3p, miR-29b-3p, miR-29c-3p, miR-30a-5p, miR-30b-5p, miR-30c-5p, miR-30d-5p, miR-30e-5p, miR-320-3p, miR-34a-5p, miR-351-5p, miR-369-3p, miR-374-5p, miR-381-3p, miR-410-3p, miR-429, miR-449a-5p, miR-539-5p, miR-664-3p, miR-673-5p, miR-743b-3p, or miR-98-5p.

For example, a microarray chip can consist essentially of oligonucleotides corresponding to: miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p & miR-19b, miR-101, miR-144, miR448, miR-760-5p, miR-136, miR-141, miR-291a-3p, miR-327, miR-495, miR-136, miR-145, miR-205, or a combination thereof. In another example, a microarray chip can consist essentially of oligonucleotides corresponding to: miR-181, miR-221, miR-222, miR-93, miR-200c, miR-17-5p, miR-203, miR-20a, miR-20b-5p, miR-21, miR-325-3p, miR-9, miR-27a, miR-19a, miR-497, miR-34c, miR-384-5p & miR-19b, miR-101, miR-144, miR448, miR-760-5p, miR-136, miR-141, miR-495, miR-136, miR-145, miR-205, or a combination thereof.

Further embodiments of the current invention provide microarray chips consisting essentially of oligonucleotides corresponding to miRNAs belonging to a profile of up-regulated/over-expressed miRNAs in a lymphatic vessel cell under a proinflammatory stimulus. For example, a microarray chip can consist essentially of oligonucleotides corresponding to one or more of miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p, and miR-19b. In another example, a microarray chip can consist essentially of oligonucleotides corresponding to one or more of miR-181, miR-221, miR-222, miR-93, miR-200c, miR-17-5p, miR-203, miR-20a, miR-20b-5p, miR-21, miR-325-3p, miR-9, miR-27a, miR-19a, miR-497, miR-34c, miR-384-5p, and miR-19b.

Even further embodiments of the current invention provide microarray chips consisting essentially of oligonucleotides corresponding to miRNAs belonging to a profile of down-regulated/under-expressed miRNAs in a lymphatic vessel cell under a proinflammatory stimulus. For example, a microarray chip can consist essentially of oligonucleotides corresponding to one or more of miR-101, miR-144, miR-20a, miR448, miR-760-5p, miR-136, miR-141, miR-291a-3p, miR-327, miR-495, miR-136, miR-145 and miR-205. In another example, a microarray chip can consist essentially of oligonucleotides corresponding to one or more of miR-101, miR-144, miR-20a, miR448, miR-760-5p, miR-136, miR-141, miR-495, miR-136, miR-145 and miR-205.

A lymphatic vessel cell expresses a specific set of miRNAs that regulate several critical pathways underlying inflammation, angiogenesis, epithelial to mesenchymal transition (EMT), endothelial to mesenchymal transition (EndMT), cell proliferation, and cellular senescence. Certain embodiments of the current invention provide microarray chips consisting essentially of oligonucleotides corresponding to miRNAs belonging to a set of differentially expressed miRNAs in a lymphatic vessel cell under a proinflammatory stimulus, wherein the differentially expressed miRNAs are involved in a particular response, for example, angiogenesis, epithelial to mesenchymal transition, endothelial to mesenchymal transition, cell proliferation, cellular senescence, cell proliferation, vascular remodeling, adipose metabolism, and inflammatory signaling. For example, a microarray chip can consist essentially of oligonucleotides corresponding to a set of miRNAs involved in inflammation (miR-9, miR-21), angiogenesis (miR-20a, miR-20b-5p, miR-21, miR-9, miR-145, miR-27a, miR-17-5p, miR-322, miR-19b), EMT/EndMT (miR-141, miR-200c, miR-136, miR-21, miR-9), cellular senescence (miR-34a, miR-34c) and cell proliferation (miR-203, miR-141, miR-17-5p). miRNAs in the profile of differentially expressed miRNAs in a lymphatic vessel cell under a proinflammatory stimulus target a distinct group of genes involved in diverse cellular processes including endothelial cellular senescence, endothelial mesenchymal transition, cell proliferation, vascular remodeling, adipose metabolism, and inflammatory signaling.

Therefore, miRNAs in these profiles can be used as biomarkers for diagnosis of inflammation-mediated lymphatic diseases. For example, alterations in the expression of miRNAs that belong to the profile of miRNAs differentially expressed in a lymphatic vessel cell under a proinflammatory stimulus can be indicative of inflammatory diseases in lymphatic tissues of a subject. Further, the alterations in expression of miRNAs that regulate a particular pathway involved in inflammation, angiogenesis, epithelial to mesenchymal transition (EMT), endothelial to mesenchymal transition (EndMT), cell proliferation, or cellular senescence would indicate the activation of these pathways.

Certain embodiments of the current invention provide a method of screening a subject for an inflammation-mediated lymphatic disease, the method comprising:

a) obtaining a tissue sample from the subject,

b) obtaining a reference sample,

c) determining the expression of an miRNA in the tissue sample and the reference sample, wherein the miRNA belongs to a profile of differentially expressed miRNAs in a lymphatic vessel cell under a proinflammatory stimulus,

d) comparing the expression of the miRNA in the tissue sample with the expression of the miRNA in the reference sample, and

e) determining the presence of the inflammation-mediated lymphatic disease in the subject if the miRNA is differentially expressed in the tissue sample as compared to the reference sample.

The reference sample can be obtained from an organism not having the inflammation-mediated lymphatic disease. The reference sample can also be obtained from the subject at a time point when the subject was known to be free from the inflammation-mediated lymphatic disease. The organism and the subject can be a mammal, for example, a human, an ape, a pig, a bovine, or a feline.

The lymphatic vessel cell can be a lymphatic endothelial cell or a lymphatic muscle cell.

The miRNA that can be tested according to the methods of the current invention can be miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p and miR-19b, miR-101, miR-144, miR-20a, miR448, miR-760-5p, miR-136, miR-141, miR-291a-3p, miR-327, miR-495, miR-136, miR-144, miR-145, and miR-205.

In an embodiment, the method of screening a subject for an inflammation-mediated lymphatic disease comprises determining the expression of a plurality of miRNAs, wherein each miRNA belongs to the profile of differentially expressed miRNAs in a lymphatic vessel cell under a proinflammatory stimulus. For example, a plurality of miRNAs can be selected from miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p and miR-19b, miR-101, miR-144, miR-20a, miR448, miR-760-5p, miR-136, miR-141, miR-291a-3p, miR-327, miR-495, miR-136, miR-144, miR-145, and miR-205.

Certain embodiments of the current invention provide methods of screening a subject for activation of specific pathway in a lymphatic vessel cell in response to a proinflammatory stimulus, the method comprising:

a) obtaining a tissue sample from the subject,

b) obtaining a reference sample,

c) determining the expression of an miRNA in the tissue sample and the reference sample, wherein the miRNA is involved in the activation of the pathway in the lymphatic vessel cell,

d) comparing the expression of the miRNA in the tissue sample with the expression of the miRNA in the reference sample, and

e) determining the activation of the pathway in the subject if the miRNA is differentially expressed in the tissue sample as compared to the reference sample.

The pathway can be angiogenesis, epithelial to mesenchymal transition, endothelial to mesenchymal transition, cell proliferation, cellular senescence, cell proliferation, vascular remodeling, adipose metabolism, or inflammation.

The reference sample can be obtained from an organism not having a particular pathway activated. The reference sample can also be obtained from the subject at a time point when the subject was known to be free from the activation of the particular pathway.

The organism and the subject can be a mammal, for example, a human, an ape, a pig, a bovine, or a feline.

As mentioned above, differential expression of miRNAs in lymphatic vessel cells in response to a proinflammatory stimulus is associated with development of inflammation-mediated lymphatic diseases. Consequently, these miRNAs provide ideal drug targets for treating the inflammation-mediated lymphatic diseases. The drugs can be oligonucleotides.

Additional embodiments of the current invention provide methods of treating inflammation-mediated lymphatic diseases by modulating the expression or activity of an miRNA differentially expressed in a lymphatic vessel cell under a proinflammatory stimulus expression profile.

Treating an inflammation-mediated lymphatic disease by modulating the expression or activity of a differentially expressed miRNA can be achieved by administering to a subject in need thereof a pharmaceutically effective amount of an agent capable of activating or inhibiting the expression and/or activity of the miRNA. The agent can be an antagomir of the miRNA, a mimic of the miRNA, or the miRNA.

The miRNA antagomirs, miRNA mimics, or miRNAs for treatment of inflammation-mediated lymphatic diseases can be directed to one or more of miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p and miR-19b, miR-101, miR-144, miR-20a, miR448, miR-760-5p, miR-136, miR-141, miR-291a-3p, miR-327, miR-495, miR-136, miR-144, miR-145, and miR-205.

An embodiment of the invention provides a method of treating inflammation-mediated lymphatic diseases by modulating the expression or activity of miR-9 in a subject, the method comprising administering to the subject a pharmaceutically effective amount of an agent capable of activating and/or inhibiting the expression and/or activity of miR-9. The agent can be an miR-9 antagomir, a miR-9 mimic, or miR-9 itself.

The agent capable of modulating the expression and/or activity of an miRNA can be administered to the subject as a pharmaceutical composition comprising the agent and a pharmaceutically acceptable carrier. If the agent is an oligonucleotide, it can also be administered in the form of an expression vector that encodes the oligonucleotide upon entry into the cells of the subject. Various techniques of preparing vectors expressing an oligonucleotide or miRNA and their administration to a subject in need thereof are well known to a person of ordinary skill in the art and such techniques are within the purview of this invention.

Further embodiments of the current invention provide a composition comprising an agent and a pharmaceutically acceptable carrier, wherein the agent is capable of modulating expression/activity of an miRNA which belongs to a profile of miRNAs differentially expressed in a lymphatic vessel cell under a proinflammatory stimulus. The agent can be an miRNA antagomir, an miRNA mimic, or miRNA itself.

The agent can be directed to one or more of miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p and miR-19b, miR-101, miR-144, miR-20a, miR448, miR-760-5p, miR-136, miR-141, miR-291a-3p, miR-327, miR-495, miR-136, miR-144, miR-145, and miR-205.

An embodiment of the invention provides a composition comprising an agent and a pharmaceutically acceptable carrier, wherein the agent modulates the activity/expression of miR-9. The agent can be an miR-9 antagomir, an miR-9 mimic, or miR-9 itself.

The miRNAs in the profile of differentially expressed miRNAs in a lymphatic vessel cell under a proinflammatory stimulus target a distinct gene or genes. A target gene for a particular miRNA is a gene whose expression is directly or indirectly affected by a particular miRNA. For example, if miRNA-X changes the expression of gene-A, and the change in the expression of gene-A changes the expression of gene-B, then both gene-A and gene-B are target genes of miRNA-X. Table 1 provides a list of several miRNAs that belong to a profile of miRNAs differentially expressed in a lymphatic vessel cell under a proinflammatory stimulus and their corresponding predicted target genes identified by bioinformatics analysis using the TARGETSCAN, miRANDA, miRWALK, PICTAR5, and miRDB databases.

TABLE 1
Bioinformatic analysis of miRNAs and their predicted target genes

| miRNA | Target | Unigene ID | Name |
|---|---|---|---|
| miR-9 | BCL2L11 | Hs.469658 | BCL2-like 11 (apoptosis facilitator) |
| miR-9 | SH2B3 | Hs.506784 | SH2B adaptor protein 3 |
| miR-9 | RASGRP1 | Hs.591127 | RAS guanyl releasing protein 1 (calcium and DAG- |
| | | | regulated) |
| miR-9 | LYVE1 | Hs.655332 | lymphatic vessel endothelial hyaluronan receptor 1 |
| miR-9 | MDGA2 | Hs.436380 | MAM domain containing |
| | | | glycosylphosphatidylinositol anchor 2 |
| miR-9 | CSDA | Hs.221889 | cold shock domain protein A |
| miR-9 | SLC50A1 | Hs.292154 | solute carrier family 50, member 1 |
| miR-9 | CTNNA1 | Hs.534797 | catenin (cadherin-associated protein), alpha 1 |
| miR-9 | FBN1 | Hs.591133 | fibrillin 1 |
| miR-9 | PRDM6 | Hs.135118 | PR domain containing 6 |
| miR-9 | TGFB1 | Hs.645227 | transforming growth factor, beta-induced, 68 kDa |
| miR-9 | NOX4 | Hs.371036 | NADPH oxidase 4 |
| miR-9 | MARCH6 | Hs.432862 | membrane-associated ring finger (C3HC4) 6, E3 |
| | | | ubiquitin protein ligase |
| miR-9 | TIMM23 | Hs.524308 | translocase of inner mitochondrial membrane 23 |
| | | | homolog |
| miR-9 | ERLIN2 | Hs.705490 | ER lipid raft associated 2 |
| miR-9 | FSTL1 | Hs.269512 | follistatin-like 1 |
| miR-9 | SOCS5 | Hs.468426 | suppressor of cytokine signaling 5 |
| miR-9 | NEDD4 | Hs.1565 | neural precursor cell expressed, developmentally |
| | | | down-regulated 4, E3 ubiquitin protein ligase |
| miR-9 | OINECUT2 | Hs.194725 | one cut homeobox 2 |
| miR-9 | TGFBI | Hs.369397 | transforming growth factor, beta-induced, 68 kDa |
| miR-9 | ZAK | Hs.444451 | sterile alpha motif and leucine zipper containing |
| | | | kinase AZK |
| miR-9 | ONECUT1 | Hs.658573 | one cut homeobox 1 |
| miR-9 | MAGT1 | Hs.323562 | magnesium transporter 1 |
| miR-9 | NID2 | Hs.369840 | nidogen 2 (osteonidogen) |
| miR-9 | SLC25A43 | Hs.496658 | solute carrier family 25, member 43 |
| miR-9 | SNX25 | Hs.369091 | sorting nexin 25 |
| miR-9 | ENPEP | Hs.435765 | glutamyl aminopeptidase (aminopeptidase A) |
| miR-9 | TNC | Hs.143250 | tenascin C |
| miR-9 | FOXN2 | Hs.468478 | forkhead box N2 |
| miR-9 | SFXN2 | Hs.44070 | sideroflexin 2 |
| miR-9 | BEND3 | Hs.418045 | BEN domain containing 3 |
| miR-9 | PIGM | Hs.552810 | phosphatidylinositol glycan anchor biosynthesis, |
| | | | class M |
| miR-9 | TNFAIP8 | Hs.656274 | tumor necrosis factor, alpha-induced protein 8 |
| miR-9 | PXDN | Hs.332197 | peroxidasin homolog (Drosophila) |
| miR-9 | LEPRE1 | Hs.437656 | leucine proline-enriched proteoglycan (leprecan) 1 |
| miR-9 | MTHFD2 | Hs.469030 | methylenetetrahydrofolate dehydrogenase |
| miR-9 | KLHL18 | Hs.517946 | kelch-like 18 (Drosophila) |
| miR-9 | C2orf15 | Hs.352211 | chromosome 2 open reading frame 15 |
| miR-9 | PPM1F | Hs.112728 | protein phosphatase, Mg2+/Mn2+ dependent, 1F |
| miR-9 | DDHD2 | Hs.434966 | DDHD domain containing 2 |
| miR-9 | SGMS2 | Hs.595423 | sphingomyelin synthase 2 |
| miR-9 | KLF5 | Hs.508234 | Kruppel-like factor 5 (intestinal) |
| miR-9 | GALNT3 | Hs.170986 | UDP-N-acetyl-alpha-D-galactosamine:polypeptide |
| | | | N-acetylgalactosaminyltransferase 3 (GalNAc-T3) |
| miR-9 | PDK4 | Hs.8364 | pyruvate dehydrogenase kinase, isozyme 4 |
| miR-9 | TINAGL1 | Hs.199368 | tubulointerstitial nephritis antigen-like 1 |
| miR-9 | CCNDBP1 | Hs.36794 | cyclin D-type binding-protein 1 |
| miR-9 | VRTN | | vertebrae development homolog (pig) |
| miR-9 | CALB2 | Hs.106857 | calbindin 2 |
| miR-9 | VAV3 | Hs.267659 | vav 3 guanine nucleotide exchange factor |
| miR-9 | LEP | Hs.194236 | leptin |
| miR-9 | SLC6A2 | Hs.78036 | solute carrier family 6 (neurotransmitter transporter, |
| | | | noradrenalin), member 2 |
| miR-9 | TESK2 | Hs.591499 | testis-specific kinase 2 |
| miR-9 | CLDN14 | Hs.660278 | claudin 14 |
| miR-9 | VCAN | Hs.695930 | versican |
| miR-9 | SHC1 | Hs.433795 | SHC (Src homology 2 domain containing) |
| | | | transforming protein 1 |
| miR-9 | SHROOM4 | Hs.420541 | shroom family member 4 |
| miR-9 | SLC14A1 | Hs.101307 | solute carrier family 14 (urea transporter), member 1 |
| | | | (Kidd blood group) |
| miR-9 | PRDM1 | Hs.436023 | PR domain containing 1, with ZNF domain |
| miR-9 | DSE | Hs.458358 | dermatan sulfate epimerase |
| miR-9 | MESDC1 | Hs.513071 | mesoderm development candidate 1 |
| miR-9 | LMNA | Hs.594444 | lamin A/C |
| miR-9 | ARFGEF2 | Hs.62578 | ADP-ribosylation factor guanine nucleotide- |
| | | | exchange factor 2 (brefeldin A-inhibited) |
| miR-9 | COL9A1 | Hs.590892 | collagen, type IX, alpha 1 |
| miR-9 | ENTPD1 | Hs.576612 | ectonucleoside triphosphate diphosphohydrolase 1 |
| miR-9 | COL15A1 | Hs.409034 | collagen, type XV, alpha 1 |
| miR-9 | CCNG1 | Hs.79101 | cyclin G1 |
| miR-9 | BRAF | Hs.550061 | v-raf murine sarcoma viral oncogene homolog B1 |
| miR-9 | AP1S2 | Hs.653504 | adaptor-related protein complex 1, sigma 2 subunit |
| miR-9 | SCRIB | Hs.436329 | scribbled homolog (Drosophila) |
| miR-9 | STARD13 | Hs.507704 | StAR-related lipid transfer (START) domain |
| | | | containing 13 |
| miR-9 | C2orf88 | Hs.389311 | chromosome 2 open reading frame 88 |
| miR-9 | BAG4 | Hs.194726 | BCL2-associated athanogene 4 |
| miR-9 | FBN2 | Hs.519294 | fibrillin 2 |
| miR-9 | ZBED3 | Hs.584988 | zinc finger, BED-type containing 3 |
| miR-9 | ProSAPiP1 | Hs.90232 | ProSAPiPl protein |
| miR-9 | CXCL11 | Hs.632592 | chemokine (C-X-C motif) ligand 11 |
| miR-9 | ECHDC1 | Hs.486410 | enoyl CoA hydratase domain containing 1 |
| miR-9 | FKTN | Hs.55777 | fukutin |
| miR-9 | CEP63 | Hs.443301 | centrosomal protein 63 kDa |
| miR-9 | EMB | Hs.645309 | embigin |
| miR-9 | LECT1 | Hs.421391 | leukocyte cell derived chemotaxin 1 |
| miR-9 | BEST3 | Hs.280782 | bestrophin 3 |
| miR-9 | ITGA6 | Hs.133397 | integrin, alpha 6 |
| miR-9 | MCMBP | Hs.124246 | minichromosome maintenance complex binding |
| | | | protein |
| miR-9 | UBE3C | Hs.118351 | ubiquitin protein ligase E3C |
| miR-9 | ADAMTS3 | Hs.590919 | ADAM metallopeptidase with thrombospondin type |
| | | | 1 motif, 3 |
| miR-9 | ATP8B2 | Hs.435700 | ATPase, class I, type 8B, member 2 |
| miR-9 | AP3B1 | Hs.532091 | adaptor-related protein complex 3, beta 1 subunit |
| miR-9 | OPN3 | Hs.409081 | opsin 3 |
| miR-9 | SAMD8 | Hs.663616 | sterile alpha motif domain containing 8 |
| miR-9 | PDGFRB | Hs.509067 | platelet-derived growth factor receptor, beta |
| | | | polypeptide |
| miR-9 | RHOJ | Hs.656339 | ras homolog gene family, member J |
| miR-9 | SIRT1 | Hs.369779 | sirtuin 1 |
| miR-9 | C21orf91 | Hs.293811 | chromosome 21 open reading frame 91 |
| miR-9 | ALCAM | Hs.591293 | activated leukocyte cell adhesion molecule |
| miR-9 | ULK2 | Hs.168762 | unc-51-like kinase 2 (C. elegans) |
| miR-9 | SOCS5 | Hs.468426 | suppressor of cytokine signaling 5 |
| miR-9 | UBE3C | Hs.118351 | ubiquitin protein ligase E3C |
| miR-9 | ADAMTS3 | Hs.590919 | ADAM metallopeptidase with thrombospondin type |
| | | | 1 motif, 3 |
| miR-9 | ATP8B2 | Hs.435700 | ATPase, class I, type 8B, member 2 |
| miR-9 | AP3B1 | Hs.532091 | adaptor-related protein complex 3, beta 1 subunit |
| miR-9 | OPN3 | Hs.409081 | opsin 3 |
| miR-9 | SAMD8 | Hs.663616 | sterile alpha motif domain containing 8 |
| miR-141 | HMG20A | Hs.69594 | high mobility group 20A |
| miR-141 | DLC1 | Hs.134296 | deleted in liver cancer 1 |
| miR-141 | AKAP11 | Hs.105105 | A kinase (PRKA) anchor protein 11 |
| miR-141 | DCUN1D3 | Hs.101007 | DCN1, defective in cullin neddylation 1, domain |
| | | | containing 3 |
| miR-141 | CCDC128 | Hs.654619 | PPP1R21 protein phosphatase 1, regulatory subunit 21 |
| miR-141 | COX11 | Hs.591171 | cytochrome c oxidase assembly homolog 11 |
| miR-141 | CTBP2 | Hs.501345 | C-terminal binding protein 2 |
| miR-141 | RNF145 | FIs.349306 | ring finger protein 145 |
| miR-141 | SLC26A2 | Hs.302738 | solute carrier family 26 (anion exchanger), member 2 |
| miR-141 | E2F3 | Hs.269408 | E2F transcription factor 3 |
| miR-141 | JAZF1 | Hs.368944 | JAZF zinc finger 1 |
| miR-141 | ITGA11 | Hs.436416 | integrin, alpha 11 |
| miR-141 | DNAJC8 | Hs.433540 | DnaJ (Hsp40) homolog, subfamily C, member 8 |
| miR-141 | MON2 | Hs.389378 | MON2 homolog (S. cerevisiae) |
| miR-141 | CLASP2 | Hs.696092 | cytoplasmic linker associated protein 2 |
| miR-141 | ZFR | Hs.696117 | zinc finger RNA binding protein |
| miR-141 | RANBP6 | Hs.167496 | RAN binding protein 6 |
| miR-141 | ZEB2 | Hs.34871 | zinc finger E-box binding homeobox 2 |
| miR-141 | ABL2 | Hs.159472 | v-abl Abelson murine leukemia viral oncogene |
| | | | homolog 2 |
| miR-141 | ARPC5 | Hs.518609 | actin related protein 2/3 complex, subunit 5, 16 kDa |
| miR-141 | TMEM170B | Hs.146317 | transmembrane protein 170B |
| miR-141 | SLC35D1 | Hs.213642 | solute carrier family 35 (UDP-glucuronic acid/UDP- |
| | | | N-acetylgalactosamine dual transporter), member D1 |
| miR-141 | PRKACB | Hs.487325 | protein kinase, cAMP-dependent, catalytic, beta |
| miR-141 | FBXW2 | Hs.494985 | F-box and WD repeat domain containing 2 |
| miR-141 | PPM1E | Hs.245044 | protein phosphatase, Mg2+/Mn2+ dependent, 1E |
| miR-141 | CXCL12 | Hs.522891 | chemokine (C-X-C motif) ligand 12 |
| miR-141 | MACC1 | Hs.598388 | metastasis associated in colon cancer 1 |
| miR-141 | GNL1 | Hs.83147 | guanine nucleotide binding protein-like 1 |
| miR-141 | MYH10 | Hs.16355 | myosin, heavy chain 10, non-muscle |
| miR-141 | TGFB2 | Hs.133379 | transforming growth factor, beta 2 |
| miR-141 | SCD5 | Hs.379191 | stearoyl-CoA desaturase 5 |
| miR-141 | ELMOD1 | Hs.495779 | ELMO/CED-12 domain containing 1 |
| miR-141 | DSTYK | Hs.6874 | dual serine/threonine and tyrosine protein kinase |
| miR-141 | MAP2K4 | Hs.514681 | mitogen-activated protein kinase kinase 4 |
| miR-141 | HSPA13 | Hs.352341 | heat shock protein 70 kDa family, member 13 |
| miR-141 | KLF12 | Hs.373857 | Kruppel-like factor 12 |
| miR-141 | ATP8A1 | Hs.435052 | ATPase, aminophospholipid transporter (APLT), |
| | | | class I, type 8A, member 1 |
| miR-141 | ELAVL2 | Hs.166109 | ELAV (embryonic lethal, abnormal vision, |
| | | | Drosophila)-like 2 |
| miR-141 | EDEM1 | Hs.224616 | ER degradation enhancer, mannosidase alpha-like 1 |
| miR-141 | PLAG1 | Hs.14968 | pleiomorphic adenoma gene 1 |
| miR-141 | ACOT7 | Hs.126137 | acyl-CoA thioesterase 7 |
| miR-141 | TSC1 | Hs.370854 | tuberous sclerosis 1 |
| miR-141 | TRHDE | Hs.199814 | thyrotropin-releasing hormone degrading enzyme |
| miR-141 | LMO3 | Hs.504908 | LIM domain only 3 (rhombotin-like 2) |
| miR-141 | STRN | Hs.656726 | striatin, calmodulin binding protein |
| miR-141 | DUSP3 | Hs.695925 | dual specificity phosphatase 3 |
| miR-141 | YWHAG | Hs.520974 | tyrosine 3-monooxygenase/tryptophan 5- |
| | | | monooxygenase activation protein, gamma |
| | | | polypeptide |
| miR-141 | STAT4 | Hs.80642 | signal transducer and activator of transcription 4 |
| miR-141 | MN1 | Hs.268515 | meningioma (disrupted in balanced translocation) 1 |
| miR-141 | PGRMC2 | Hs.507910 | progesterone receptor membrane component 2 |
| miR-141 | NDFIP2 | Hs.525093 | Nedd4 family interacting protein 2 |
| miR-141 | FAM168B | Hs.534679 | family with sequence similarity 168, member B |
| miR-141 | PLEK | Hs.468840 | pleckstrin |
| miR-141 | MYBL1 | Hs.654538 | v-myb myeloblastosis viral oncogene homolog |
| | | | (avian)-like 1 |
| miR-141 | ASTN1 | Hs.495897 | astrotactin 1 |
| miR-141 | PTPRD | Hs.446083 | protein tyrosine phosphatase, receptor type, D |
| miR-141 | LSAMP | Hs.657246 | limbic system-associated membrane protein |
| miR-141 | ZFR | Hs.696117 | zinc finger RNA binding protein |
| miR-141 | RANBP6 | Hs.167496 | RAN binding protein 6 |
| miR-141 | ZEB2 | Hs.34871 | zinc finger E-box binding homeobox 2 |
| miR-141 | ABL2 | Hs.159472 | v-abl Abelson murine leukemia viral oncogene |
| | | | homolog 2 |
| miR-141 | ARPC5 | Hs.518609 | actin related protein 2/3 complex, subunit 5, 16 kDa |
| miR-322 | ZBTB34 | Hs.177633 | zinc finger and BTB domain containing 34 |
| miR-322 | PRKAR2A | Hs.631923 | protein kinase, cAMP-dependent, regulatory, type II, |
| | | | alpha |
| miR-322 | MGAT4A | Hs.177576 | mannosyl (alpha-1,3-)-glycoprotein beta-1,4-N- |
| | | | acetylglucosaminyltransferase, isozyme A |
| miR-322 | PAPPA | Hs.643599 | pregnancy-associated plasma protein A, pappalysin 1 |
| miR-322 | C20orf46 | Hs.516834 | chromosome 20 open reading frame 46 |
| miR-322 | CPEB2 | Hs.656937 | cytoplasmic polyadenylation element binding |
| | | | protein 2 |
| miR-322 | SPTBN2 | Hs.26915 | spectrin, beta, non-erythrocytic 2 |
| miR-322 | N4BP1 | Hs.511839 | NEDD4 binding protein 1 |
| miR-322 | CAPZA2 | Hs.695918 | capping protein (actin filament) muscle Z-line, alpha 2 |
| miR-322 | HTR4 | Hs.483773 | 5-hydroxytryptamine (serotonin) receptor 4 |
| miR-322 | EYA1 | Hs.491997 | eyes absent homolog 1 (Drosophila) |
| miR-322 | PWWP2B | Hs.527751 | PWWP domain containing 2B |
| miR-322 | C12orf51 | Hs.379848 | chromosome 12 open reading frame 51 |
| miR-322 | DCLK1 | Hs.507755 | doublecortin-like kinase 1 |
| miR-322 | FGF2 | Hs.284244 | fibroblast grow th factor 2 (basic) |
| miR-322 | YTHDC1 | Hs.175955 | YTH domain containing 1 |
| miR-322 | DPY19L4 | Hs.567828 | dpy-19-like 4 (C. elegans) |
| miR-322 | TRAF3 | Hs.510528 | TNF receptor-associated factor 3 |
| miR-322 | UBE2Q1 | Hs.607928 | ubiquitin-conjugating enzyme E2Q family member 1 |
| miR-322 | LUZP1 | Hs.257900 | leucine zipper protein 1 |
| miR-322 | RSBN1 | Hs.486285 | round spermatid basic protein 1 |
| miR-322 | WNT3A | Hs.336930 | wingless-type MMTV integration site family, |
| | | | member 3A |
| miR-322 | C10orf46 | Hs.420024 | chromosome 10 open reading frame 46 |
| miR-322 | CDK17 | Hs.506415 | cyclin-dependent kinase 17 |
| miR-322 | EPT1 | Hs.189073 | ethanolaminephosphotransferase 1 (CDP- |
| | | | ethanolamine-specific) |
| miR-322 | CTTNBP2NL | Hs.485899 | CTTNBP2 N-terminal like |
| miR-322 | SLC12A6 | Hs.510939 | solute carrier family 12 (potassium/chloride |
| | | | transporters), member 6 |
| miR-322 | USP31 | Hs.183817 | ubiquitin specific peptidase 31 |
| miR-322 | USP15 | Hs.434951 | ubiquitin specific peptidase 15 |
| miR-322 | OTX1 | Hs.445340 | orthodenticle homeobox 1 |
| miR-322 | HMGA1 | Hs.518805 | high mobility group AT-hook 1 |
| miR-322 | ZBTB46 | Hs.585028 | zinc finger and BTB domain containing 46 |
| miR-322 | NYNRIN | Hs.288348 | NYN domain and retroviral integrase containing |
| miR-322 | ASH1L | Hs.491060 | ash1 (absent, small, or homeotic)-like (Drosophila) |
| miR-322 | CCNE1 | Hs.244723 | cyclin E1 |
| miR-322 | ATP1B4 | Hs.662608 | ATPase, Na+/K+ transporting, beta 4 polypeptide |
| miR-322 | KIF23 | Hs.270845 | kinesin family member 23 |
| miR-322 | PHF19 | Hs.460124 | PHD finger protein 19 |
| miR-322 | PCMT1 | Hs.279257 | protein-L-isoaspartate (D-aspartate) O- |
| | | | methyltransferase |
| miR-322 | CDAN1 | Hs.599232 | congenital dyserythropoietic anemia, type I |
| miR-322 | ENTPD7 | Hs.744948 | ectonucleoside triphosphate diphosphohydrolase 7 |
| miR-322 | NUP210 | Hs.475525 | nucleoporin 210 kDa |
| miR-322 | ODZ2 | Hs.654631 | odz, odd Oz/ten-m homolog 2 (Drosophila) |
| miR-322 | ZYX | Hs.490415 | zyxin |
| miR-322 | CACNB1 | Hs.635 | calcium channel, voltage-dependent, beta 1 subunit |
| miR-322 | FAM126A | Hs.85603 | family with sequence similarity 126, member A |
| miR-322 | ELL | Hs.515260 | elongation factor RNA polymerase II |
| miR-322 | EIF4G2 | Hs.183684 | eukaryotic translation initiation factor 4 gamma, 2 |
| miR-322 | SLC7A2 | Hs.448520 | |
| miR-322 | ZBTB34 | Hs.177633 | zinc finger and BTB domain containing 34 |
| miR-322 | PRKAR2A | Hs.631923 | protein kinase, cAMP-dependent, regulatory, type II, |
| | | | alpha |
| miR-322 | MGAT4A | Hs.177576 | mannosyl (alpha-1,3-)-glycoprotein beta-1,4-N- |
| | | | acetylglucosaminvltransferase, isozyme A |
| miR-322 | PAPPA | Hs.643599 | pregnancy-associated plasma protein A, pappalysin 1 |
| miR-322 | C20orf46 | Hs.516834 | chromosome 20 open reading frame 46 |
| miR-322 | CPEB2 | Hs.656937 | cytoplasmic poly adenylation element binding |
| | | | protein 2 |
| miR-322 | SPTBN2 | Hs.26915 | spectrin, beta, non-erythrocytic 2 |
| miR-322 | N4BP1 | Hs.511839 | NEDD4 binding protein 1 |
| miR-322 | CAPZA2 | Hs.695918 | capping protein (actin filament) muscle Z-line, alpha 2 |
| miR-322 | HTR4 | Hs.483773 | 5-hydroxytryptamine (serotonin) receptor 4 |
| miR-322 | EYA1 | Hs.491997 | eyes absent homolog 1 (Drosophila) |
| miR-322 | PWWP2B | Hs.527751 | PWWP domain containing 2B |
| miR-322 | C12orf51 | Hs.379848 | chromosome 12 open reading frame 51 |
| miR-322 | DCLK1 | Hs.507755 | doublecortin-like kinase 1 |
| miR-322 | FGF2 | Hs.284244 | fibroblast growth factor 2 (basic) |
| miR-322 | YTHDC1 | Hs.175955 | YTH domain containing 1 |
| miR-322 | DPY19L4 | Hs.567828 | dpy-19-like 4 (C. elegans) |
| miR-322 | TRAF3 | Hs.510528 | TNF receptor-associated factor 3 |
| miR-322 | UBE2Q1 | Hs.607928 | ubiquitin-conjugating enzyme E2Q family member 1 |
| miR-322 | LUZP1 | Hs.257900 | leucine zipper protein 1 |
| miR-203 | MBNL2 | Hs.657347 | muscleblind-like splicing regulator 2 |
| miR-203 | IL24 | Hs.58831 | interleukin 24 |
| miR-203 | GLCCI1 | Hs.131673 | glucocorticoid induced transcript 1 |
| miR-203 | CD109 | Hs.399891 | CD109 molecule |
| miR-203 | COX15 | Hs.591916 | cytochrome c oxidase assembly homolog 15 (yeast) |
| miR-203 | DAZL | Hs.131179 | deleted in azoospermia-like |
| miR-203 | EPYC | Hs.435680 | epiphycan |
| miR-203 | RTKN2 | Hs.58559 | rhotekin 2 |
| miR-203 | MS4A2 | Hs.386748 | membrane-spanning 4-domains, subfamily A, |
| | | | member 2 |
| miR-203 | AAK1 | Hs.468878 | AP2 associated kinase 1 |
| miR-203 | PDXDC1 | Hs.370781 | pyridoxal-dependent decarboxylase domain |
| | | | containing 1 |
| miR-203 | GABRA4 | Hs.248112 | gamma-aminobutyric acid (GABA) A receptor, |
| | | | alpha 4 |
| miR-203 | RBPMS2 | Hs.436518 | RNA binding protein with multiple splicing 2 |
| miR-203 | RBJ | Hs.434993 | DnaJ (Hsp40) homolog, subfamily C, member 27 |
| miR-203 | PIK3C2A | Hs.175343 | phosphatidylinositol-4-phosphate 3-kinase, catalytic |
| | | | subunit type 2 alpha |
| miR-203 | PLD2 | Hs.104519 | phospholipase D2 |
| miR-203 | ZNF281 | Hs.59757 | zinc finger protein 281 |
| miR-203 | CAMTA1 | Hs.397705 | calmodulin binding transcription activator 1 |
| miR-203 | B3GNT5 | Hs.208267 | UDP-GlcNAc:betaGal beta-1,3-N- |
| | | | acetylglucosaminyltransferase 5 |
| miR-203 | LIFR | Hs.133421 | leukemia inhibitory factor receptor alpha |
| miR-203 | ABCE1 | Hs.12013 | ATP-binding cassette, sub-family E (OABP), |
| | | | member 1 |
| miR-203 | NUDT21 | Hs.528834 | nudix (nucleoside diphosphate linked moiety X)- |
| | | | type motif 21 |
| miR-203 | AFF4 | Hs.519313 | AF4/FMR2 family, member 4 |
| miR-203 | PRPS2 | Hs.654581 | phosphoribosyl pyrophosphate synthetase 2 |
| miR-203 | SEMA5A | Hs.27621 | sema domain, seven thrombospondin repeats (type 1 |
| | | | and type 1-like), transmembrane domain (TM) and |
| | | | short cytoplasmic domain (semaphorin) 5A |
| miR-203 | SMAD9 | Hs.123119 | SMAD family member 9 |
| miR-203 | SLC4A4 | Hs.5462 | solute carrier family 4, sodium bicarbonate |
| | | | cotransporter, member 4 |
| miR-203 | PHLDA1 | Hs.602085 | pleckstrin homology-like domain, family A, member 1 |
| miR-203 | UBR1 | Hs.591121 | ubiquitin protein ligase E3 component n-recognin 1 |
| miR-203 | CASK | Hs.495984 | calcium/calmodulin-dependent serine protein kinase |
| | | | (MAGUK family) |
| miR-203 | COL4A4 | Hs.591645 | collagen, type IV, alpha 4 |
| miR-203 | KCNQ5 | Hs.675919 | potassium voltage-gated channel, KQT-like |
| | | | subfamily, member 5 |
| miR-203 | TTC39A | Hs.112949 | tetratricopeptide repeat domain 39A |
| miR-203 | FAM116A | Hs.91085 | family with sequence similarity 116, member A |
| miR-203 | KCNK10 | Hs.592299 | potassium channel, subfamily K, member 10 |
| miR-203 | ADPGK | Hs.654636 | ADP-dependent glucokinase |
| miR-203 | C4orf33 | Hs.567679 | chromosome 4 open reading frame 33 |
| miR-203 | MORF4L1 | Hs.374503 | mortality factor 4 like 1 |
| miR-203 | EIF5A2 | Hs.164144 | eukaryotic translation initiation factor 5A2 |
| miR-203 | WDFY3 | Hs.480116 | WD repeat and FYVE domain containing 3 |
| miR-203 | CCDC50 | Hs.478682 | coiled-coil domain containing 50 |
| miR-203 | SEC62 | Hs.744859 | SEC62 homolog (S. cerevisiae) |
| miR-203 | CSN2 | Hs.2242 | casein beta |
| miR-203 | COX15 | Hs.591916 | COX15 homolog, cytochrome c oxidase assembly |
| | | | protein |
| miR-203 | GRHL3 | Hs.657920 | grainyhead-like 3 (Drosophila) |
| miR-203 | ELL2 | Hs.592742 | elongation factor, RNA polymerase II, 2 |
| miR-203 | HCCS | Hs.211571 | holocytochrome c synthase |
| miR-203 | RAPGEF1 | Hs.127897 | Rap guanine nucleotide exchange factor (GEF) 1 |
| miR-203 | MORF4L2 | Hs.326387 | mortality factor 4 like 2 |
| miR-203 | ROBO2 | Hs.13305 | roundabout, axon guidance receptor, homolog 2 |
| | | | (Drosophila) |
| miR-203 | ADAMTS6 | Hs.482291 | ADAM metallopeptidase with thrombospondin type |
| | | | 1 motif, 6 |
| miR-203 | TXNDC16 | Hs.532609 | thioredoxin domain containing 16 |
| miR-203 | IL24 | Hs.58831 | interleukin 24 |
| miR-203 | PRICKLE2 | Hs.699317 | prickle homolog 2 (Drosophila) |
| miR-203 | VWA3B | Hs.269977 | von Willebrand factor A domain containing 3B |
| miR-203 | COPS7B | Hs.335061 | COP9 constitutive photomorphogenic homolog |
| | | | subunit 7B (Arabidopsis) |
| miR-203 | STEAP1 | Hs.61635 | six transmembrane epithelial antigen of the prostate 1 |
| miR-203 | RAP2A | Hs.508480 | RAP2A, member of RAS oncogene family |
| miR-203 | DPY19L4 | Hs.567828 | dpy-19-like 4 (C. elegans) |
| miR-203 | HDX | Hs.559546 | highly divergent homeobox |
| miR-203 | BCL7A | Hs.530970 | B-cell CLL/lymphoma 7A |
| miR-203 | ZNF281 | Hs.59757 | zinc finger protein 281 |
| miR-203 | CAMTA1 | Hs.397705 | calmodulin binding transcription activator 1 |
| miR-203 | B3GNT5 | Hs.208267 | UDP-GlcNAc:betaGal beta-1,3-N- |
| | | | acetylglucosaminyltransferase 5 |
| miR-203 | LIFR | Hs.133421 | leukemia inhibitory factor receptor alpha |
| miR-203 | ABCE1 | Hs.12013 | ATP-binding cassette, sub-family E (OABP), |
| | | | member 1 |
| miR-203 | NUDT21 | Hs.528834 | nudix (nucleoside diphosphate linked moiety X)- |
| | | | type motif 21 |
| miR-203 | AFF4 | Hs.519313 | AF4/FMR2 family, member 4 |
| miR-203 | PRPS2 | Hs.654581 | phosphoribosyl pyrophosphate synthetase 2 |
| miR-203 | SEMA5A | Hs.27621 | sema domain, seven thrombospondin repeats (type 1 |
| | | | and type 1-like), transmembrane domain (TM) and |
| | | | short cytoplasmic domain (semaphorin) 5A |
| miR-203 | SMAD9 | Hs.123119 | SMAD family member 9 |
| miR-203 | SLC4A4 | Hs.5462 | solute carrier family 4, sodium bicarbonate |
| | | | cotransporter, member 4 |
| miR-203 | PHLDA1 | Hs.602085 | pleckstrin homology-like domain, family A, member 1 |
| miR-203 | UBR1 | Hs.591121 | ubiquitin protein ligase E3 component n-recognin 1 |
| miR-203 | CASK | Hs.495984 | calcium/calmodulin-dependent serine protein kinase |
| miR-203 | COL4A4 | Hs.591645 | collagen, type IV, alpha 4 |
| miR-20a | GNB5 | Hs.155090 | guanine nucleotide binding protein (G protein), beta 5 |
| miR-20a | POLQ | Hs.241517 | polymerase (DNA directed), theta |
| miR-20a | PAPOLA | Hs.253726 | poly(A) polymerase alpha |
| miR-20a | PTPN21 | Hs.437040 | protein tyrosine phosphatase, non-receptor type 21 |
| miR-20a | BTN3A1 | Hs.191510 | butyrophilin, subfamily 3, member A1 |
| miR-20a | GALNT6 | Hs.505575 | |
| miR-20a | KLF12 | Hs.373857 | Kruppel-like factor 12 |
| miR-20a | STK38 | Hs.409578 | serine/threonine kinase 38 |
| miR-20a | CENTD1 | Hs.479451 | ArfGAP with RhoGAP domain, ankyrin repeat and |
| | | | PH domain 2 |
| miR-20a | IKIP | Hs.252543 | IKBKB interacting protein |
| miR-20a | NIPA1 | Hs.511797 | non imprinted in Prader-Willi/Angelman syndrome 1 |
| miR-20a | NUP35 | Hs.180591 | nucleoporin 35 kDa |
| miR-20a | GNPDA2 | Hs.21398 | glucosamine-6-phosphate deaminase 2 |
| miR-20a | MUM1L1 | Hs.592221 | melanoma associated antigen (mutated) 1-like 1 |
| miR-20a | RUNDC1 | Hs.632255 | RUN domain containing 1 |
| miR 20a/20b | FGD4 | Hs.117835 | FYVE, RhoGEF and PH domain containing 4 |
| miR 20a/20b | PKD2 | Hs.181272 | polycystic kidney disease 2 (autosomal dominant) |
| miR 20a/20b | MAP3K2 | Hs.145605 | mitogen-activated protein kinase kinase kinase 2 |
| miR 20a/20b | ZNFX1 | Hs.371794 | zinc finger, NFX1-type containing 1 |
| miR 20a/20b | PDCD1LG2 | Hs.532279 | programmed cell death 1 ligand 2 |
| miR 20a/20b | PLEKHA3 | Hs.41086 | pleckstrin homology domain containing, family A |
| | | | (phosphoinositide binding specific) member 3 |
| miR 20a/20b | EIF5A2 | Hs.164144 | eukaryotic translation initiation factor 5A2 |
| miR 20a/20b | FYCO1 | Hs.200227 | FYVE and coiled-coil domain containing 1 |
| miR 20a/20b | GPR6 | Hs.46332 | G protein-coupled receptor 6 |
| miR 20a/20b | ENPP5 | Hs.35198 | ectonucleotide pyrophosphatase/phosphodiesterase 5 |
| | | | (putative) |
| miR 20a/20b | EPELA4 | Hs.371218 | EPH receptor A4 |
| miR 20a/20b | VSX1 | Hs.274264 | visual system homeobox 1 |
| miR 20a/20b | STK17B | Hs.88297 | serine/threonine kinase 17b |
| miR 20a/20b | SACS | Hs.159492 | spastic ataxia of Charlevoix-Saguenay (sacsin) |
| miR 20a/20b | C14orf28 | Hs.82098 | chromosome 14 open reading frame 28 |
| miR 20a/20b | ZFYVE26 | Hs.98041 | zinc finger, FYVE domain containing 26 |
| miR 20a/20b | RPS6KA5 | Hs.510225 | ribosomal protein S6 kinase, 90 kDa, polypeptide 5 |
| miR 20a/20b | C11orf30 | Hs.352588 | chromosome 11 open reading frame 30 |
| miR 20a/20b | XRN1 | Hs.435103 | 5′-3′ exoribonuclease 1 |
| miR 20a/20b | FBXL5 | Hs.705407 | F-box and leucine-rich repeat protein 5 |
| miR 20a/20b | CAMTA1 | Hs.397705 | calmodulin binding transcription activator 1 |
| miR 20a/20b | ITPRIPL2 | Hs.530899 | inositol 1,4,5-trisphosphate receptor interacting |
| | | | protein-like 2 |
| miR 20a/20b | GPR137C | Hs.416214 | G protein-coupled receptor 137C |
| miR 20a/20b | FTSJD1 | Hs.72782 | FtsJ methyltransferase domain containing 1 |
| miR 20a/20b | EPHA5 | Hs.654492 | EPH receptor A5 |
| miR 20a/20b | GUCY1A3 | Hs.24258 | guanylate cyclase 1, soluble, alpha 3 |
| miR 20a/20b | RRAGD | Hs.485938 | Ras-related GTP binding D |
| miR 20a/20b | GNPDA2 | Hs.21398 | glucosamine-6-phosphate deaminase 2 |
| miR 20a/20b | FBXO48 | Hs.164117 | F-box protein 48 |
| miR 20a/20b | DYNC1LI2 | Hs.369068 | dynein, cytoplasmic 1, light intermediate chain 2 |
| miR 20a/20b | FAM129A | Hs.518662 | family with sequence similarity 129, member A |
| miR 20a/20b | FIBIN | Hs.705612 | fin bud initiation factor homolog (zebrafish) |
| miR 20a/20b | EZH1 | Hs.194669 | enhancer of zeste homolog 1 (Drosophila) |
| miR 20a/20b | RNF128 | Hs.496542 | ring finger protein 128 |
| miR 20a/20b | IRF9 | Hs.1706 | interferon regulatory factor 9 |
| miR 20a/20b | DDHD1 | Hs.513260 | DDHD domain containing 1 |
| miR 20a/20b | ANKRD29 | Hs.374774 | ankyrin repeat domain 29 |
| miR 20a/20b | REST | Hs.631513 | RE1-silencing transcription factor |
| miR 20a/20b | FAM40B | Hs.489988 | family with sequence similarity 40, member B |
| miR 20a/20b | PPP1R3B | Hs.458513 | protein phosphatase 1, regulatory (inhibitor) subunit |
| | | | 3B |
| miR 20a/20b | RAB11FIPS | Hs.24557 | RAB11 family interacting protein 5 (class I) |
| miR 20a/20b | ARID4B | Hs.533633 | AT rich interactive domain 4B (RBPl-like) |
| miR 20a/20b | C2CD2 | Hs.473894 | C2 calcium-dependent domain containing 2 |
| miR 20a/20b | PRRG1 | Hs.190341 | proline rich Gla (G-carboxyglutamic acid) 1 |
| miR 20a/20b | TNFRSF21 | Hs.443577 | tumor necrosis factor receptor superfamily, member |
| | | | 21 |
| miR 20a/20b | SGTB | Hs.482301 | small glutamine-rich tetratricopeptide repeat (TPR)- |
| | | | containing, beta |
| miR 20a/20b | SEMA4B | Hs.474935 | sema domain, immunoglobulin domain (Ig), |
| | | | transmembrane domain (TM) and short cytoplasmic |
| | | | domain, (semaphorin) 4B |
| miR 20a/20b | LAMA3 | Hs.436367 | laminin, alpha 3 |
| miR 20a/20b | PTPN4 | Hs.469809 | protein tyrosine phosphatase, non-receptor type 4 |
| | | | (megakaryocyte) |
| miR 20a/20b | FGD4 | Hs.117835 | FYVE, RhoGEF and PH domain containing 4 |
| miR 20a/20b | PKD2 | Hs.181272 | polycystic kidney disease 2 (autosomal dominant) |
| miR 20a/20b | MAP3K2 | Hs.145605 | mitogen-activated protein kinase kinase kinase 2 |
| miR 20a/20b | ZNFX1 | Hs.371794 | zinc finger, NFX1-type containing 1 |
| miR 20a/20b | PDCD1LG2 | Hs.532279 | programmed cell death 1 ligand 2 |
| miR 20a/20b | PLEKHA3 | Hs.41086 | pleckstrin homology domain containing, family A |
| | | | (phosphoinositide binding specific) member 3 |
| miR 20a/20b | EIF5A2 | Hs.164144 | eukaryotic translation initiation factor 5A2 |
| miR 20a/20b | FYCO1 | Hs.200227 | FYVE and coiled-coil domain containing 1 |
| miR 20a/20b | GPR6 | Hs.46332 | G protein-coupled receptor 6 |
| miR 20a/20b | ENPP5 | Hs.35198 | ectonucleotide pyrophosphatase/phosphodiesterase 5 |
| | | | (putative) |
| miR 20a/20b | EPHA4 | Hs.371218 | EPH receptor A4 |
| miR 20a/20b | VSX1 | Hs.274264 | visual system homeobox 1 |
| miR 20a/20b | STK17B | Hs.88297 | serine/threonine kinase 17b |
| miR 20a/20b | SACS | Hs.159492 | spastic ataxia of Charlevoix-Saguenay (sacsin) |
| miR-19ab | ZMYND11 | Hs.292265 | zinc finger, MYND-type containing 11 |
| miR-19ab | LRP2 | Hs.657729 | low density lipoprotein receptor-related protein 2 |
| miR-19ab | ADRB1 | Hs.695932 | adrenergic, beta-1-, receptor |
| miR-19ab | LONRF1 | Hs.180178 | LON peptidase N-terminal domain and ring finger 1 |
| miR-19ab | DSEL | Hs.124673 | dermatan sulfate epimerase-like |
| miR-19ab | CDS1 | Hs.654899 | CDP-diacylglycerol synthase (phosphatidate |
| | | | cytidylyltransferase) 1 |
| miR-19ab | PPP1R12A | Hs.49582 | protein phosphatase 1, regulatory (inhibitor) subunit |
| | | | 12A |
| miR-19ab | TRIM23 | Hs.792 | tripartite motif containing 23 |
| miR-19ab | KCNA4 | Hs.592002 | potassium voltage-gated channel, shaker-related |
| | | | subfamily, member 4 |
| miR-19ab | CHIC1 | Hs.496323 | cysteine-rich hydrophobic domain 1 |
| miR-19ab | RAB8B | Hs.389733 | RAB8B, member RAS oncogene family |
| miR-19ab | PMEPA1 | Hs.517155 | prostate transmembrane protein, androgen induced 1 |
| miR-19ab | S1PR1 | Hs.154210 | sphingosine-1-phosphate receptor 1 |
| miR-19ab | HSPC159 | Hs.372208 | galectin-related protein |
| miR-19ab | ACBD5 | Hs.530597 | acyl-CoA binding domain containing 5 |
| miR-19ab | SEMA4C | Hs.516220 | sema domain, immunoglobulin domain (Ig), |
| | | | transmembrane domain (TM) and short cytoplasmic |
| | | | domain, (semaphorin) 4C |
| miR-19ab | TXK | Hs.479669 | TXK tyrosine kinase |
| miR-19ab | SECISBP2L | Hs.9997 | SECIS binding protein 2-like |
| miR-19ab | CLIP4 | Hs.122927 | CAP-GLY domain containing linker protein family, |
| | | | member 4 |
| miR-19ab | HBP1 | Hs.162032 | HMG-box transcription factor 1 |
| miR-19ab | MDFIC | Hs.427236 | MyoD family inhibitor domain containing |
| miR-19ab | ARHGEF26 | Hs.240845 | Rho guanine nucleotide exchange factor (GEF) 26 |
| miR-19ab | LDLR | Hs.213289 | low density lipoprotein receptor |
| miR-19ab | ZNF831 | Hs.473204 | zinc finger protein 831 |
| miR-19ab | MKL2 | Hs.592047 | MKL/myocardin-like 2 |
| miR-19ab | KPNA6 | Hs.470588 | karyopherin alpha 6 (importin alpha 7) |
| miR-19ab | SHCBP1 | Hs.123253 | SHC SH2-domain binding protein 1 |
| miR-19ab | C10orf140 | Hs.350848 | chromosome 10 open reading frame 140 |
| miR-19ab | PAK6 | Hs.513645 | p21 protein (Cdc42/Rac)-activated kinase 6 |
| miR-19ab | CAB39L | Hs.87159 | calcium binding protein 39-like |
| miR-19ab | SPOCK1 | Hs.654695 | sparc/osteonectin, cwcv and kazal-like domains |
| | | | proteoglycan (testican) 1 |
| miR-19ab | SLC24A3 | Hs.654790 | solute carrier family 24 (sodium/potassium/calcium |
| | | | exchanger), member 3 |
| miR-19ab | ZNF238 | Hs.69997 | zinc finger protein 238 |
| miR-19ab | KIAA1211 | Hs.596667 | KIAA1211 |
| miR-19ab | SYT1 | Hs.310545 | synaptotagmin I |
| miR-19ab | QKI | Hs.510324 | quaking homolog, KH domain RNA binding |
| | | | (mouse) |
| miR-19ab | LRIG3 | Hs.253736 | leucine-rich repeats and immunoglobulin-like |
| | | | domains 3 |
| miR-19ab | ZPLD1 | Hs.352213 | zona pellucida-like domain containing 1 |
| miR-19ab | BMPR2 | Hs.471119 | bone morphogenetic protein receptor, type II |
| | | | (serine/threonine kinase) |
| miR-19ab | HCFC2 | Hs.506558 | host cell factor C2 |
| miR-19ab | MDM4 | Hs.702385 | Mdm4 p53 binding protein homolog (mouse) |
| miR-19ab | LIMCH1 | Hs.335163 | LIM and calponin homology domains 1 |
| miR-19ab | SIN3B | Hs.13999 | SINS homolog B, transcription regulator (yeast) |
| miR-19ab | RIN2 | Hs.472270 | Ras and Rab interactor 2 |
| miR-19ab | SGK1 | Hs.510078 | serum/glucocorticoid regulated kinase 1 |
| miR-19ab | ATG14 | Hs.414809 | ATG14 autophagy related 14 homolog (S. |
| | | | cerevisiae) |
| miR-19ab | NEUROD1 | Hs.440955 | neurogenic differentiation 1 |
| miR-19ab | RAP2C | Hs.119889 | RAP2C, member of RAS oncogene family |
| miR-19ab | SLC26A4 | Hs.571246 | solute carrier family 26, member 4 |
| miR-19ab | GJA1 | Hs.74471 | gap junction protein, alpha 1, 43 kDa |
| miR-19ab | AFF1 | Hs.480190 | AF4/FMR2 family, member 1 |
| miR-19ab | FAM116A | Hs.91085 | family with sequence similarity 116, member A |
| miR-19ab | PATL1 | Hs.591960 | protein associated with topoisomerase II homolog 1 |
| | | | (yeast) |
| miR-19ab | SYT6 | Hs.370963 | synaptotagmin VI |
| miR-19ab | KCNJ2 | Hs.1547 | potassium inwardly-rectifying channel, subfamily I, |
| | | | member 2 |
| miR-19ab | PRUNE2 | Hs.262857 | prune homolog 2 (Drosophila) |
| miR-19ab | RORA | Hs.695914 | RAR-related orphan receptor A |
| miR-19ab | SLC9A6 | Hs.62185 | solute carrier family 9 (sodium/hydrogen |
| | | | exchanger), member 6 |
| miR-19ab | SIVA1 | Hs.l 12058 | SIVA1, apoptosis-inducing factor |
| miR-19ab | ZBTB11 | Hs.655286 | zinc finger and BTB domain containing 11 |
| miR-19ab | TNFAIP3 | Hs.591338 | tumor necrosis factor, alpha-induced protein 3 |
| miR-19ab | CNGA3 | Hs.234785 | cyclic nucleotide gated channel alpha 3 |
| miR-19ab | MON2 | Hs.389378 | MON2 homolog (S. cerevisiae) |
| miR-19ab | TSC1 | Hs.370854 | tuberous sclerosis 1 |
| miR-19ab | SLC35F1 | Hs.654841 | solute carrier family 35, member F1 |
| miR-19ab | ZMYND11 | Hs.292265 | zinc finger, MYND-type containing 11 |
| miR-17-5p | ZNFX1 | Hs.371794 | zinc finger, NFX1-type containing 1 |
| miR-17-5p | ZFYVE9 | Hs.532345 | zinc finger, FYVE domain containing 9 |
| miR-17-5p | EPHA4 | Hs.371218 | EPH receptor A4 |
| miR-17-5p | TBC1D8B | Hs.351798 | TBC1 domain family, member 8B (with GRAM |
| | | | domain) |
| miR-17-5p | IL25 | Hs.302036 | interleukin 25 |
| miR-17-5p | MAP3K2 | Hs.145605 | mitogen-activated protein kinase kinase kinase 2 |
| miR-17-5p | PLEKHA3 | Hs.41086 | pleckstrin homology domain containing, family A |
| | | | (phosphoinositide binding specific) member 3 |
| miR-17-5p | SLC40A1 | Hs.643005 | solute carrier family 40 (iron-regulated transporter), |
| | | | member 1 |
| miR-17-5p | FYCO1 | Hs.200227 | FYVE and coiled-coil domain containing 1 |
| miR-17-5p | ZFYVE26 | Hs.98041 | zinc finger, FYVE domain containing 26 |
| miR-17-5p | AMPD3 | Hs.501890 | adenosine monophosphate deaminase 3 |
| miR-17-5p | GPR137C | Hs.416214 | G protein-coupled receptor 137C |
| miR-17-5p | ZNF238 | Hs.69997 | zinc finger protein 238 |
| miR-17-5p | C11orf30 | Hs.352588 | chromosome 11 open reading frame 30 |
| miR-17-5p | DDHD1 | Hs.513260 | DDHD domain containing 1 |
| miR-17-5p | MFAP3L | Hs.593942 | microfibrillar-associated protein 3-like |
| miR-17-5p | FTSJD1 | Hs.72782 | FtsJ methyltransferase domain containing 1 |
| miR-17-5p | SLITRK3 | Hs.101745 | SLIT and NTRK-like family, member 3 |
| miR-17-5p | TRIP11 | Hs.632339 | thyroid hormone receptor interactor 11 |
| miR-17-5p | CLIP4 | Hs.122927 | CAP-GLY domain containing linker protein family, |
| | | | member 4 |
| miR-17-5p | PRRG1 | Hs.190341 | proline rich Gla (G-carboxyglutamic acid) 1 |
| miR-17-5p | SSH2 | Hs.654754 | slingshot homolog 2 (Drosophila) |
| miR-17-5p | KLHL28 | Hs.653206 | kelch-like 28 (Drosophila) |
| miR-17-5p | ARID4B | Hs.533633 | AT rich interactive domain 4B (RBP1-like) |
| miR-17-5p | MFN2 | Hs.695980 | mitofusin 2 |
| miR-17-5p | RPS6KA5 | Hs.510225 | ribosomal protein S6 kinase, 90 kDa, polypeptide 5 |
| miR-17-5p | BTG3 | Hs.473420 | BTG family, member 3 |
| miR-17-5p | ZNF367 | Hs.494557 | zinc finger protein 367 |
| miR-17-5p | SEMA7A | Hs.24640 | semaphorin 7A, GPI membrane anchor |
| miR-17-5p | SEMA4B | Hs.474935 | semaphorin) 4B |
| miR-17-5p | PLS1 | Hs.203637 | plastin 1 |
| miR-17-5p | FZD3 | Hs.40735 | frizzled family receptor 3 |
| miR-17-5p | ARHGAP12 | Hs.499264 | Rho GTPase activating protein 12 |
| miR-17-5p | KIF23 | Hs.270845 | kinesin family member 23 |
| miR-17-5p | VLDLR | Hs.370422 | very low density lipoprotein receptor |
| miR-17-5p | FBXO48 | Hs.164117 | F-box protein 48 |
| miR-17-5p | ZNF652 | Hs.463375 | zinc finger protein 652 |
| miR-17-5p | RASD1 | Hs.25829 | RAS, dexamethasone-induced 1 |
| miR-17-5p | TNFRSF21 | Hs.443577 | tumor necrosis factor receptor superfamily, member |
| | | | 21 |
| miR-17-5p | PTPN4 | Hs.469809 | protein tyrosine phosphatase, non-receptor type 4 |
| miR-17-5p | NANOS1 | Hs.591918 | nanos homolog 1 (Drosophila) |
| miR-17-5p | FJX1 | Hs.39384 | four jointed box 1 (Drosophila) |
| miR-17-5p | EZH1 | Hs.194669 | enhancer of zeste homolog 1 (Drosophila) |
| miR-17-5p | E2F5 | Hs.445758 | E2F transcription factor 5, p130-binding |
| miR-17-5p | PGM2L1 | Hs.26612 | phosphoglucomutase 2-like 1 |
| miR-17-5p | MAP3K8 | Hs.432453 | mitogen-activated protein kinase kinase kinase 8 |
| miR-17-5p | MASTL | Hs.276905 | microtubule associated serine/threonine kinase-like |
| miR-17-5p | VSX1 | Hs.274264 | visual system homeobox 1 |
| miR-17-5p | ANO6 | Hs.505339 | anoctamin 6 |
| miR-17-5p | FRMD6 | Hs.434914 | FERM domain containing 6 |
| miR-17-5p | UNC80 | Hs.396201 | unc-80 homolog (C. elegans) |
| miR-17-5p | NKIRAS1 | Hs.173202 | NFKB inhibitor interacting Ras-like 1 |
| miR-145 | TPM3 | Hs.535581 | tropomyosin 3 |
| | FSCN1 | Hs.118400 | fascin homolog 1, actin-bundling protein |
| | | | (Strongylocentrotus purpuratus) |
| miR-145 | SRGAP2 | Hs.497575 | SLIT-ROBO Rho GTPase activating protein 2 |
| miR-145 | FAM108C1 | Hs.459072 | family with sequence similarity 108, member C1 |
| miR-145 | NAV3 | Hs.655301 | neuron navigator 3 |
| miR-145 | ABCE1 | Hs.12013 | ATP-binding cassette, sub-family E, member 1 |
| miR-145 | KCNA4 | Hs.592002 | potassium voltage-gated channel, shaker-related |
| | | | subfamily, member 4 |
| miR-145 | PLDN | Hs.7037 | pallidin homolog (mouse) |
| miR-145 | FLI1 | Hs.504281 | Friend leukemia virus integration 1 |
| miR-145 | SEMA3A | Hs.252451 | sema domain, immunoglobulin domain (Ig), short |
| | | | basic domain, secreted, (semaphorin) 3A |
| miR-145 | CSRNP2 | Hs.524425 | cysteine-serine-rich nuclear protein 2 |
| miR-145 | YTHDF2 | Hs.532286 | YTH domain family, member 2 |
| miR-145 | DAB2 | Hs.481980 | disabled homolog 2, mitogen-responsive |
| | | | phosphoprotein (Drosophila) |
| miR-145 | TMEM9B | Hs.501853 | TMEM9 domain family, member B |
| miR-145 | MPZL2 | Hs.116651 | myelin protein zero-like 2 |
| miR-145 | ADD3 | Hs.501012 | adducin 3 (gamma) |
| miR-145 | ST6GALNAC3 | Hs.337040 | ST6 (alpha-N-acetyl-neuraminyl-2,3-beta- |
| | | | galactosyl-1,3)-N-acetylgalactosaminide alpha-2,6- |
| | | | sialyltransferase 3 |
| miR-145 | SRGAP1 | Hs.450763 | SLIT-ROBO Rho GTPase activating protein 1 |
| miR-145 | EXOC8 | Hs.356198 | exocyst complex component 8 |
| miR-145 | TRIM2 | Hs.435711 | tripartite motif containing 2 |
| miR-145 | MYO5A | Hs.21213 | myosin VA (heavy chain 12, myoxin) |
| miR-145 | ITGB8 | Hs.592171 | integrity beta 8 |
| miR-145 | CAMSAP1L1 | Hs.23585 | calmodulin regulated spectrin-associated protein 1- |
| | | | like 1 |
| miR-145 | ATXN2 | Hs.76253 | ataxin 2 |
| miR-145 | HHEX | Hs.118651 | hematopoietically expressed homeobox |
| miR-145 | ZBTB33 | Hs.143604 | zinc finger and BTB domain containing 33 |
| miR-145 | ARL11 | Hs.558599 | ADP-ribosylation factor-like 11 |
| miR-145 | SLC24A4 | Hs.510281 | solute carrier family 24 |
| miR-145 | FNDC3A | Hs.508010 | fibronectin type III domain containing 3A |
| miR-145 | GLCE | Hs.183006 | glucuronic acid epimerase |
| miR-145 | MYLK4 | Hs.127830 | myosin light chain kinase family, member 4 |
| miR-145 | RBPMS2 | Hs.436518 | RNA binding protein with multiple splicing 2 |
| miR-145 | IYD | Hs.310225 | iodotvrosine deiodinase |
| miR-145 | MFAP3 | Hs.432818 | microfibrillar-associated protein 3 |
| miR-145 | CLK4 | Hs.406557 | CDC-like kinase 4 |
| miR -878-3p | SLC16A1 | Hs.75231 | solute carrier family 16, member 1 |
| miR -878-3p | AKAP6 | Hs.509083 | A kinase (PRKA) anchor protein 6 |
| miR -878-3p | PAQR9 | Hs.408385 | progestin and adipoQ receptor family member IX |
| miR -878-3p | TOP2B | Hs.475733 | topoisomerase (DNA) II beta 180 kDa |
| miR -878-3p | LIFR | Hs.133421 | leukemia inhibitory factor receptor alpha |
| miR -878-3p | NRIP1 | Hs.155017 | nuclear receptor interacting protein 1 |
| miR -878-3p | COPS2 | Hs.369614 | COP9 constitutive photomorphogenic homolog |
| | | | subunit 2 (Arabidopsis) |
| miR -878-3p | PAFAH1B1 | Hs.77318 | platelet-activating factor acetylhydrolase 1b, |
| | | | regulatory subunit 1 (45 kDa) |
| miR -878-3p | CXCL6 | Hs.164021 | chemokine (C-X-C motif) ligand 6 (granulocyte |
| | | | chemotactic protein 2) |
| miR -878-3p | LRRC49 | Hs.12692 | leucine rich repeat containing 49 |
| miR -878-3p | FLG2 | Hs.156124 | filaggrin family member 2 |
| miR -878-3p | CMTM4 | Hs.699299 | CKLF-like MARVEL transmembrane domain |
| | | | containing 4 |
| miR -878-3p | TOX3 | Hs.460789 | TOX high mobility group box family member 3 |
| miR -878-3p | KRAS | Hs.505033 | v-Ki-ras2 Kirsten rat sarcoma viral oncogene |
| | | | homolog |
| miR -878-3p | ARL8B | Hs.250009 | ADP-ribosylation factor-like 8B |
| miR -878-3p | PABPN1 | Hs.117176 | poly(A) binding protein, nuclear 1 |
| miR -878-3p | PABPN1 | Hs.707712 | BCL2L2-PABPN1 readthrough |
| miR -878-3p | CXCL5 | Hs.89714 | chemokine (C-X-C motif) ligand 5 |
| miR -878-3p | WWC3 | Hs.527524 | WWC family member 3 |
| miR -878-3p | ZNF292 | Hs.590890 | zinc finger protein 292 |
| miR -878-3p | C11orf73 | Hs.283322 | chromosome 11 open reading frame 73 |
| miR -878-3p | YWHAG | Hs.520974 | tyrosine 3-monooxygenase/tryptophan 5- |
| | | | monooxygenase activation protein, gamma |
| | | | polypeptide |
| miR -878-3p | PDK3 | Hs.658190 | pyruvate dehydrogenase kinase, isozyme 3 |
| miR -878-3p | ZNF608 | Hs.266616 | zinc finger protein 608 |
| miR -878-3p | MPP7 | Hs.499159 | membrane protein, palmitoylated 7 |
| miRNA | Target | Unigene Id | Name (Predicted from Rat) |
| miR-448 | KCNQ4 | Hs.473058 | potassium voltage-gated channel, member 4 |
| miR-448 | LHFP | Hs.507798 | lipoma HMGIC fusion partner |
| miR-448 | NBEA | Hs.491172 | neurobeachin |
| miR-448 | KLF5 | Hs.508234 | Kmppel-like factor 5 (intestinal) |
| miR-448 | BCL2 | Hs.150749 | B-cell CLL/lymphoma 2 |
| miR-448 | FBXO33 | Hs.324342 | F-box protein 33 |
| miR-448 | DENND1B | Hs.657779 | DENN/MADD domain containing 1B |
| miR-448 | KCNMB2 | Hs.478368 | potassium large conductance calcium-activated |
| | | | channel, subfamily M, beta member 2 |
| miR-448 | IRS2 | Hs.442344 | insulin receptor substrate 2 |
| miR-448 | CPEB2 | Hs.656937 | cytoplasmic polyadenylation element binding |
| | | | protein 2 |
| miR-448 | ZNF711 | Hs.326801 | zinc finger protein 711 |
| miR-448 | KCTD9 | Hs.72071 | potassium channel tetramerisation domain |
| | | | containing 9 |
| miR-448 | AZIN1 | Hs.459106 | antizyme inhibitor 1 |
| miR-448 | TMEM55A | Hs.202517 | transmembrane protein 55A |
| miR-448 | C21orf91 | Hs.293811 | chromosome 21 open reading frame 91 |
| miR-448 | DOC2A | Hs.355281 | double C2-like domains, alpha |
| miR-448 | SRSF10 | Hs.3530 | serine/arginine-rich splicing factor 10 |
| miR-448 | GEM | Hs.654463 | GTP binding protein overexpressed in skeletal |
| | | | muscle |
| miR-448 | RAB2B | Hs.22399 | RAB2B, member RAS oncogene family |
| miR-448 | WRNIP1 | Hs.236828 | Werner helicase interacting protein 1 |
| miR-448 | C5orf13 | Hs.36053 | chromosome 5 open reading frame 13 |
| miR-448 | CLCN5 | Hs.166486 | chloride channel 5 |
| miR-448 | DTX3 | Hs.32374 | deltex homolog 3 (Drosophila) |
| miR-448 | NRG3 | Hs.125119 | neuregulin 3 |
| miR-448 | GRHL3 | Hs.657920 | grainy head-like 3 (Drosophila) |
| miR-448 | HECW2 | Hs.654742 | HECT, C2 and WW domain containing E3 ubiquitin |
| | | | protein ligase 2 |
| miR-448 | TCEAL1 | Hs.95243 | transcription elongation factor A (SII)-like 1 |
| miR-448 | PHF3 | Hs.348921 | PHD finger protein 3 |
| miR-448 | DNAJB11 | Hs.317192 | DnaJ (Hsp40) homolog, subfamily B, member 11 |
| miR-448 | DCAF5 | Hs.509780 | DDB 1 and CUL4 associated factor 5 |
| miR-448 | PHTF1 | Hs.655824 | putative homeodomain transcription factor 1 |
| miR-448 | WWP1 | Hs.655189 | WW domain containing E3 ubiquitin protein ligase 1 |
| miR-448 | PCDH8 | Hs.19492 | protocadherin 8 |
| miR-448 | FOXN2 | Hs.468478 | forkhcad box N2 |
| miR-448 | ZZEF1 | Hs.277624 | zinc finger, ZZ-type with EF-hand domain 1 |
| miR-448 | SLAIN2 | Hs.479677 | SLAIN motif family, member 2 |
| miR-448 | SMURF1 | Hs.189329 | SMAD specific E3 ubiquitin protein ligase 1 |
| miR-448 | KCNH7 | Hs.657413 | potassium voltage-gated channel, subfamily H, |
| | | | member 7 |
| miR-448 | SOCS5 | Hs.468426 | suppressor of cytokine signaling 5 |
| miR-448 | HDLBP | Hs.471851 | high density lipoprotein binding protein |
| miR-448 | MFHAS1 | Hs.379414 | malignant fibrous histiocytoma amplified sequence 1 |
| miR-448 | SESTD1 | Hs.591613 | SEC 14 and spectrin domains 1 |
| miR-448 | TPCN1 | Hs.524763 | two pore segment channel 1 |
| miR-448 | BCL11A | Hs.370549 | B-cell CLL/lymphoma 11A |
| miR-448 | ARNTL | Hs.65734 | aryl hydrocarbon receptor nuclear translocator-like |
| miR-448 | GLCE | Hs.183006 | glucuronic acid epimerase |
| miR-448 | XYLT2 | Hs.699363 | xylosyltransferase II |
| miR-448 | LYST | Hs.532411 | lysosomal trafficking regulator |
| miR-448 | SLC26A7 | Hs.354013 | solute carrier family 26, member 7 |
| miR-448 | BACH2 | Hs.269764 | BTB and CNC homology 1, basic leucine zipper |
| | | | transcription factor 2 |
| miR-448 | RANBP2 | Hs.199561 | RAN binding protein 2 |
| miR-448 | VEZF1 | Hs.705368 | vascular endothelial zinc finger 1 |
| miR-448 | CAP1 | Hs.370581 | CAP, adenylate cyclase-associated protein 1 |
| miR-448 | YAF2 | Hs.699313 | YY1 associated factor 2 |
| miR-448 | GAN | Hs.112569 | gigaxonin |
| miR-448 | GNAI1 | Hs.134587 | guanine nucleotide binding protein (G protein), |
| | | | alpha inhibiting activity polypeptide 1 |
| miR-448 | PPAPDC3 | Hs.134292 | phosphatidic acid phosphatase type 2 domain |
| | | | containing 3 |
| miR-448 | LRRC57 | Hs.234681 | leucine rich repeat containing 57 |
| miR-448 | PFN2 | Hs.91747 | profilin 2 |
| miR-448 | HEY2 | Hs.144287 | hairy/enhancer-of-split related with YRPW motif 2 |
| miR-448 | AUTS2 | Hs.700600 | autism susceptibility candidate 2 |
| miR-448 | OPTX2 | Hs.288655 | orthodenticle homeobox 2 |
| miR-448 | KANK4 | Hs.283398 | KN motif and ankyrin repeat domains 4 |
| miR-448 | ROBO2 | Hs.13305 | roundabout, axon guidance receptor, homolog 2 |
| | | | (Drosophila) |
| miR-448 | DPP6 | Hs.490684 | dipeptidyl-peptidase 6 |
| miR-448 | RBM26 | Hs.558528 | RNA binding motif protein 26 |
| miR-448 | PHKB | Hs.78060 | phosphorylase kinase, beta |
| miR-448 | ADD1 | Hs.183706 | adducin 1 (alpha) |
| miR-448 | PDP1 | Hs.22265 | pyruvate dehyrogenase phosphatase catalytic subunit 1 |
| miR-448 | PRKAB2 | Hs.50732 | protein kinase, AMP-activated, beta 2 non-catalytic |
| | | | subunit |
| miR-448 | SYN1 | Hs.225936 | synapsin I |
| miR-448 | PACRG | Hs.25791 | PARK2 co-regulated |
| miR-448 | SERTAD2 | Hs.693696 | SERTA domain containing 2 |
| miR-448 | GPHN | Hs.208765 | gephyrin |
| miR-448 | OPN5 | Hs.213717 | opsin 5 |
| miR-448 | EPHA4 | Hs.371218 | EPH receptor A4 |
| miRNA | Target * | | Name |
| miR-384-5p | MKRN3 | Hs.72964 | makorin ring finger protein 3 |
| miR-384-5p | CELSR3 | Hs.631926 | cadherin, EGF LAG seven-pass G-type receptor 3 |
| | | | (flamingo homolog, Drosophila) |
| miR-384-5p | CYP24A1 | Hs.89663 | cytochrome P450, family 24, subfamily A, |
| | | | polypeptide 1 |
| miR-384-5p | NAALADL2 | Hs.565848 | N-acetvlated alpha-linked acidic dipeptidase-like 2 |
| miR-384-5p | EED | Hs.503510 | embryonic ectoderm development |
| miR-384-5p | ACTC1 | Hs.705440 | actin, alpha, cardiac muscle 1 |
| miR-384-5p | TWF1 | Hs.189075 | twinfilin, actin-binding protein, homolog 1 |
| | | | (Drosophila) |
| miR-384-5p | FOXG1 | Hs.695962 | forkhead box G1 |
| miR-384-5p | POLR3G | Hs.282387 | polymerase (RNA) III (DNA directed) polypeptide |
| | | | G (32 kD) |
| miR-384-5p | KLHL20 | Hs.495035 | kelch-like 20 (Drosophila) |
| miR-384-5p | PDE7A | Hs.584788 | phosphodiesterase 7A |
| miR-384-5p | PPARGC1B | Hs.591261 | peroxisome proliferator-activated receptor gamma, |
| | | | coactivator 1 beta |
| miR-384-5p | RUNX2 | Hs.535845 | runt-related transcription factor 2 |
| miR-384-5p | GALNT7 | Hs.548088 | UDP-N-acetyl-alpha-D-galactosamine:polypeptide |
| | | | N-acetylgalactosaminyltransferase 7 |
| miR-384-5p | MTDH | Hs.377155 | metadherin |
| miR-384-5p | CPSF6 | Hs.369606 | cleavage and polyadenylation specific factor 6, |
| | | | 68 kDa |
| miR-384-5p | PTP4A1 | Hs.227777 | protein tyrosine phosphatase type IVA, member 1 |
| miR-384-5p | NT5E | Hs.153952 | 5′-nuclcotidase, ecto (CD73) |
| miR-384-5p | GABRB1 | Hs.27283 | gamma-aminobutyric acid (GABA) A receptor, beta 1 |
| miR-384-5p | SGCB | Hs.438953 | sarcoglycan, beta |
| miR-384-5p | TNRC6A | Hs.655057 | trinucleotide repeat containing 6A |
| miR-384-5p | CCNE2 | Hs.567387 | cyclin E2 |
| miR-384-5p | ZDHHC21 | Hs.649522 | zinc finger, DHHC-type containing 21 |
| miR-384-5p | CADPS | Hs.654933 | Ca++-dependent secretion activator |
| miR-384-5p | RFX6 | Hs.352276 | regulatory factor X, 6 |
| miR-384-5p | PTPN13 | Hs.436142 | protein tyrosine phosphatase, non-receptor type 13 |
| | | | (APO-1/CD95 (Fas)-associated phosphatase) |
| miR-384-5p | LARP1B | Hs.657067 | La ribonucleoprotein domain family, member 1B |
| miR-384-5p | SOX9 | Hs.700579 | SRY (sex determining region Y)-box 9 |
| miR-384-5p | ALS2CR8 | Hs.444982 | amyotrophic lateral sclerosis 2 (juvenile) |
| | | | chromosome region, candidate 8 |
| miR-384-5p | FAM46A | Hs.10784 | family with sequence similarity 46, member A |
| miR-384-5p | KLHL28 | Hs.653206 | kelch-like 28 (Drosophila) |
| miR-384-5p | NAP1L5 | Hs.12554 | nucleosome assembly protein 1-like 5 |
| miR-384-5p | KIAA1715 | Hs.209561 | KIAA1715 |
| miR-384-5p | VAT1L | Hs.461405 | vesicle amine transport protein 1 homolog (T. |
| | | | califomica)-like |
| miR-384-5p | RAPGEF4 | Hs.470646 | Rap guanine nucleotide exchange factor (GEF) 4 |
| miR-384-5p | TMEM181 | Hs.99145 | transmembrane protein 181 |
| miR-384-5p | R3HDM1 | Hs.412462 | R3H domain containing 1 |
| miR-384-5p | ZNRF1 | Hs.427284 | zinc and ring finger 1 |
| miR-384-5p | FAM49A | Hs.467769 | family with sequence similarity 49, member A |
| miR-384-5p | HNRNPUL2 | Hs.657058 | heterogeneous nuclear ribonucleoprotein U-like 2 |
| miR-384-5p | ANKHD1 | Hs.653135 | ankyrin repeat and KH domain containing 1 |
| miR-384-5p | SGMS2 | Hs.595423 | sphingomyelin synthase 2 |
| miR-384-5p | CPNE8 | Hs.40910 | copine VIII |
| miR-384-5p | KIAA2026 | Hs.535060 | KIAA2026 |
| miR-384-5p | JOSD1 | Hs.3094 | Josephin domain containing 1 |
| miR-384-5p | RAB15 | Hs.512492 | RAB15, member RAS onocogene family |
| miR-384-5p | LHX8 | Hs.403934 | LIM homeobox 8 |
| miR-384-5p | ABL1 | Hs.431048 | c-abl oncogene 1, non-receptor tyrosine kinase |
| miR-384-5p | SRSF7 | Hs.309090 | serine/arginine-rich splicing factor 7 |
| miR-384-5p | SYNGR3 | Hs.435277 | synaptogyrin 3 |
| miR-384-5p | IDH1 | Hs.593422 | isocitrate dehydrogenase 1 (NADP+), soluble |
| miR-384-5p | CAPN7 | Hs.631920 | calpain 7 |
| miR-384-5p | USP44 | Hs.646421 | ubiquitin specific peptidase 44 |
| miR-384-5p | RARG | Hs.1497 | retinoic acid receptor, gamma |
| miR-384-5p | GALNT3 | Hs.170986 | UDP-N-acetyl-alpha-D-galactosamine:polypeptide |
| | | | N-acetylgalactosaminyltransferase 3 |
| miR-384-5p | ABI3BP | Hs.477015 | ABI family, member 3 (NESH) binding protein |
| miR-384-5p | SCN2A | Hs.93485 | sodium channel, voltage-gated, type II, alpha subunit |
| miR-384-5p | NR4A2 | Hs.563344 | nuclear receptor subfamily 4, group A, member 2 |
| miR-384-5p | OMG | Hs.113874 | oligodendrocyte myelin glycoprotein |
| miR-384-5p | PCSK1 | Hs.78977 | proprotein convertase subtilisin/kexin type 1 |
| miR-384-5p | CRHBP | Hs.115617 | corticotropin releasing hormone binding protein |
| miR-384-5p | ANKRA2 | Hs.239154 | ankyrin repeat, family A (RFXANK-like), 2 |
| miR-384-5p | CADPS | Hs.654933 | Ca++-dependent secretion activator |
| miR-384-5p | PDCL | Hs.271749 | phosducin-like |
| miR-325-3p | ZCCHC5 | Hs.134873 | zinc finger, CCHC domain containing 5 |
| miR-325-3p | PNPT1 | Hs.388733 | polyribonucleotide nucleotidyltransferase 1 |
| miR-325-3p | SNN | Hs.700592 | stannin |
| miR-325-3p | KIF20B | Hs.240 | kinesin family member 20B |
| miR-325-3p | LMCD1 | Hs.475353 | LIM and cysteine-rich domains 1 |
| miR-325-3p | VSIG1 | Hs.177164 | V-set and immunoglobulin domain containing 1 |
| miR-325-3p | LRRC19 | Hs.128071 | leucine rich repeat containing 19 |
| miR-325-3p | GNB4 | Hs.173030 | guanine nucleotide binding protein (G protein), beta |
| | | | polypeptide 4 |
| miR-325-3p | LRRTM4 | Hs.285782 | leucine rich repeat transmembrane neuronal 4 |
| miR-325-3p | STXBP5L | Hs.477315 | syntaxin binding protein 5-like |
| miR-325-3p | LARP IB | Hs.657067 | La ribonucleoprotein domain family, member 1B |
| miR-325-3p | POSTN | Hs.136348 | periostin, osteoblast specific factor |
| miR-325-3p | CSN2 | Hs.2242 | casein beta |
| miR-325-3p | SATB2 | Hs.516617 | SATB homeobox 2 |
| miR-325-3p | KY | Hs.146730 | kyphoscoliosis peptidase |
| miR-325-3p | CTBP2 | Hs.501345 | C-terminal binding protein 2 |
| miR-325-3p | TNKS | Hs.370267 | tankyrase, TRF1-interacting ankyrin-related ADP- |
| | | | ribose polymerase |
| miR-325-3p | ERBB4 | Hs.390729 | v-erb-a erythroblastic leukemia viral oncogene |
| | | | homolog 4 (avian) |
| miR-325-3p | FOXK2 | Hs.696000 | forkhead box K2 |
| miR-325-3p | FOXO4 | Hs.584654 | forkhead box O4 |
| miR-325-3p | PDGFRA | Hs.74615 | platelet-derived growth factor receptor, alpha |
| | | | polypeptide |
| miR-325-3p | IGF2R | Hs.487062 | insulin-like growth factor 2 receptor |
| miR-325-3p | RBPMS | Hs.334587 | RNA binding protein with multiple splicing |
| miR-325-3p | ARHGEF9 | Hs.54697 | Cdc42 guanine nucleotide exchange factor (GEF) 9 |
| miR-325-3p | SEC24A | Hs.595540 | SEC24 family, member A (S. cerevisiae) |
| miR-325-3p | ZNF654 | Hs.591650 | zinc finger protein 654 |
| miR-325-3p | ADCY6 | Hs.525401 | adenylate cyclase 6 |
| miR-325-3p | NRBF2 | Hs.449628 | nuclear receptor binding factor 2 |
| miR-325-3p | RUNX2 | Hs.535845 | runt-related transcription factor 2 |
| miR-325-3p | ARPP21 | Hs.475902 | cAMP-regulated phosphoprotein, 21 kDa |
| miR-325-3p | UCK1 | Hs.9597 | uridine-cytidine kinase 1 |
| miR-325-3p | OIT3 | Hs.8366 | oncoprotein induced transcript 3 |
| miR-325-3p | AKAP6 | Hs.509083 | A kinase (PRKA) anchor protein 6 |
| miR-325-3p | TCEA1 | Hs.491745 | transcription elongation factor A (SII), 1 |
| miR-325-3p | INPP5E | Hs.120998 | inositol polyphosphate-5-phosphatase, 72 kDa |
| miR-325-3p | RBMS3 | Hs.696468 | RNA binding motif, single stranded interacting |
| | | | protein 3 |
| miR-325-3p | ANKHD1 | Hs.653135 | ankyrin repeat and KH domain containing 1 |
| miR-325-3p | SLC10A3 | Hs.522826 | solute carrier family 10, member 3 |
| miR-325-3p | IFT74 | Hs.145402 | intraflagellar transport 74 homolog |
| | | | (Chlamydomonas) |
| miR-325-3p | CCDC77 | Hs.631656 | coiled-coil domain containing 77 |
| miR-325-3p | NCKAP5 | | NCK-associated protein 5 |
| miR-325-3p | TBX3 | Hs.129895 | T-box 3 |
| miR-325-3p | TMEM199 | | transmembrane protein 199 |
| miR-325-3p | SERPINB4 | Hs.123035 | serpin peptidase inhibitor, clade B (ovalbumin), |
| | | | member 4 |
| miR-325-3p | PHLPP2 | | PH domain and leucine rich repeat protein |
| | | | phosphatase 2 |
| miR-325-3p | COL11A1 | Hs.523446 | collagen, type XI, alpha 1 |
| miR-325-3p | RLF | Hs.205627 | rearranged L-myc fusion |
| miR-325-3p | MBTPS1 | Hs.75890 | membrane-bound transcription factor peptidase, site 1 |
| miR-325-3p | COL12A1 | Hs.101302 | collagen, type XII, alpha 1 |
| miR-325-3p | EHMT1 | Hs.495511 | euchromatic histone-lysine N-methyltransferase 1 |
| miR-325-3p | PRRX1 | Hs.702224 | paired related homeobox 1 |
| miR-325-3p | HLTF | Hs.3068 | helicase-like transcription factor |
| miR-325-3p | NADK | Hs.654792 | NAD kinase |
| miR-325-3p | LPGAT1 | Hs.654626 | lysophosphatidylglycerol acyltransferase 1 |
| miR-325-3p | NUDT21 | Hs.528834 | nudix (nucleoside diphosphate linked moiety X)- |
| | | | type motif 21 |
| miR-325-3p | PDCD10 | Hs.478150 | programmed cell death 10 |
| miR-325-3p | PRSS22 | Hs.459709 | protease, serine, 22 |
| miR-325-3p | DIDO1 | Hs.517172 | death inducer-obliterator 1 |
| miR-325-3p | PKP1 | Hs.497350 | plakophilin 1 (ectodennal dysplasia/skin fragility |
| | | | syndrome) |
| miR-325-3p | IMPDH2 | Hs.654400 | IMP (inosine 5′-monophosphate) dehydrogenase 2 |
| miR-325-3p | SBDS | Hs.110445 | Shwachman-Bodian-Diamond syndrome |
| miR-325-3p | IGSF11 | Hs.112873 | immunoglobulin superfamily, member 11 |
| miR-325-3p | ZNF275 | Hs.348963 | zinc finger protein 275 |
| miR-325-3p | HYOU1 | Hs.277704 | hypoxia up-regulated 1 |
| miR-325-3p | ZCCHC5 | Hs.134873 | zinc finger, CCHC domain containing 5 |
| miR-325-3p | PNPT1 | Hs.388733 | polyribonucleotide nucleotidyltransferase 1 |
| miR-325-3p | SNN | Hs.700592 | stannin |
| miR-325-3p | KIF20B | | kinesin family member 20B |
| miR-325-3p | LMCD1 | Hs.475353 | LIM and cysteine-rich domains 1 |
| miR-325-3p | VSIG1 | Hs.177164 | V-set and immunoglobulin domain containing 1 |
| miR-325-3p | LRRC19 | Hs.128071 | leucine rich repeat containing 19 |
| miR-325-3p | GNB4 | Hs.173030 | guanine nucleotide binding protein (G protein), beta |
| | | | polypeptide 4 |
| miR-325-3p | LRRTM4 | Hs.285782 | leucine rich repeat transmembrane neuronal 4 |
| miR-325-3p | STXBP5L | Hs.477315 | syntaxin binding protein 5-like |
| miR-325-3p | LARP1B | | La ribonucleoprotein domain family, member 1B |
| miR-325-3p | POSTN | Hs.136348 | periostin, osteoblast specific factor |
| miR-325-3p | CSN2 | Hs.2242 | casein beta |
| miR-325-3p | SATB2 | Hs.516617 | SATB homeobox 2 |
| miR-325-3p | KY | Hs.146730 | kyphoscoliosis peptidase |
| miR-325-3p | CTBP2 | Hs.501345 | C-terminal binding protein 2 |
| miR-325-3p | TNKS | Hs.370267 | tankyrase, TRF1-interacting ankyrin-related ADP- |
| | | | ribose polymerase |
| miR-325-3p | ERBB4 | Hs.390729 | v-erb-a erythroblastic leukemia viral oncogene |
| | | | homolog 4 (avian) |
| miR-325-3p | FOXK2 | Hs.696000 | forkhead box K2 |
| miR-325-3p | FOXO4 | Hs.584654 | forkhead box O4 |
| miR-325-3p | PDGFRA | Hs.74615 | platelet-derived growth factor receptor, alpha |
| | | | polypeptide |
| miRNA | Target | | Name |
| miR 221/222 | SNX4 | Hs.507243 | sorting nexin 4 |
| miR 221/222 | RGS6 | Hs.509872 | regulator of G-protein signaling 6 |
| miR 221/222 | CDKN1B | Hs.238990 | cyclin-dependent kinase inhibitor 1B (p27, Kip1) |
| miR 221/222 | CXCL12 | Hs.522891 | chemokine (C-X-C motif) ligand 12 |
| miR 221/222 | TCF12 | Hs.511504 | transcription factor 12 |
| miR 221/222 | RFX7 | | regulatory factor X, 7 |
| miR 221/222 | OSTM1 | Hs.226780 | osteopetrosis associated transmembrane protein 1 |
| miR 221/222 | SEC62 | | SEC62 homolog (S. cerevisiae) |
| miR 221/222 | ZNF181 | Hs.659191 | zinc finger protein 181 |
| miR 221/222 | MYBL1 | Hs.654538 | v-myb myeloblastosis viral oncogene homolog |
| | | | (avian)-like 1 |
| miR 221/222 | GABRA1 | Hs.175934 | gamma-aminobutyric acid (GABA) A receptor, |
| | | | alpha 1 |
| miR 221/222 | BEAN1 | Hs.97805 | brain expressed, associated with NEDD4, 1 |
| miR 221/222 | HECTD2 | Hs.656960 | HECT domain containing 2 |
| miR 221/222 | PHACTR4 | Hs.225641 | phosphatase and actin regulator 4 |
| miR 221/222 | KIT | Hs.479754 | v-kit Hardy-Zuckerman 4 feline sarcoma viral |
| | | | oncogene homolog |
| miR 221/222 | VGLL4 | Hs.373959 | vestigial like 4 (Drosophila) |
| miR 221/222 | KCNQ3 | Hs.374023 | potassium voltage-gated channel, KQT-like |
| | | | subfamily, member 3 |
| miR 221/222 | WSB2 | Hs.506985 | WD repeat and SOCS box containing 2 |
| miR 221/222 | WDR35 | Hs.205427 | WD repeat domain 35 |
| miR 221/222 | PAIP1 | Hs.482038 | poly(A) binding protein interacting protein 1 |
| miR 221/222 | CPEB3 | Hs.131683 | cytoplasmic polyadenylation element binding |
| | | | protein 3 |
| miR 221/222 | TP53BP2 | Hs.523968 | tumor protein p53 binding protein, 2 |
| miR 221/222 | NAP1L5 | Hs.12554 | nucleosome assembly protein 1-like 5 |
| miR 221/222 | ZNF25 | Hs.499429 | zinc finger protein 25 |
| miR 221/222 | ZFAND5 | Hs.406096 | zinc finger, AN1-type domain 5 |
| miR 221/222 | NRK | Hs.209527 | Nik related kinase |
| miR 221/222 | IRX5 | Hs.435730 | iroquois homeobox 5 |
| miR 221/222 | PANK3 | Hs.591729 | pantothenate kinase 3 |
| miR 221/222 | PLXNC1 | Hs.584845 | plexin C1 |
| miR 221/222 | PCMTD1 | Hs.308480 | protein-L-isoaspartate (D-aspartate) O- |
| | | | methyltransferase domain containing 1 |
| miR 221/222 | DCUN1D1 | Hs.104613 | DCN1, defective in cullin neddylation 1, domain |
| | | | containing 1 (S. cerevisiae) |
| miR 221/222 | CLVS2 | Hs.486361 | clavesin 2 |
| miR 221/222 | LRRTM2 | Hs.656653 | leucine rich repeat transmembrane neuronal 2 |
| miR 221/222 | ETV3 | Hs.352672 | ets variant 3 |
| miR 221/222 | AP3B2 | Hs.199593 | adaptor-related protein complex 3, beta 2 subunit |
| miR 221/222 | PPP3R1 | Hs.280604 | protein phosphatase 3, regulatory subunit B, alpha |
| miR 221/222 | RIMS3 | Hs.654808 | regulating synaptic membrane exocytosis 3 |
| miR 221/222 | DCAF12 | Hs.493750 | DDB1 and CUL4 associated factor 12 |
| miR 221/222 | TMSB15B | Hs.675540 | thymosin beta 15B |
| miR 221/222 | C11orf41 | Hs.502266 | chromosome 11 open reading frame 41 |
| miR 221/222 | ZNF652 | Hs.463375 | zinc finger protein 652 |
| miR 221/222 | DMRT3 | Hs.189174 | doublesex and mab-3 related transcription factor 3 |
| miR 221/222 | CCDC64 | Hs.369763 | coiled-coil domain containing 64 |
| miR 221/222 | EML6 | Hs.656692 | echinoderm microtubule associated protein like 6 |
| miR 221/222 | CACNB4 | Hs.614033 | calcium channel, voltage-dependent, beta 4 subunit |
| miR 221/222 | PLEKHA2 | Hs.369123 | pleckstrin homology domain containing, family A |
| | | | (phosphoinositide binding specific) member 2 |
| miR 221/222 | FUSIP1 | Hs.3530 | serine/arginine-rich splicing factor 10 |
| miR 221/222 | ZNF572 | Hs.175350 | zinc finger protein 572 |
| miR 221/222 | CXADR | Hs.705503 | coxsackie virus and adenovirus receptor |
| miR 221/222 | AMZ1 | Hs.42221 | archaelysin family metallopeptidase 1 |
| miR 221/222 | HIPK1 | Hs.532363 | homeodomain interacting protein kinase 1 |
| miR 221/222 | RSBN1L | Hs.72451 | round spermatid basic protein 1-like |
| miR 221/222 | CHSY1 | Hs.110488 | chondroitin sulfate synthase 1 |
| miR 221/222 | RAB18 | Hs.406799 | member RAS oncogene family |
| miR 221/222 | DNM3 | Hs.654775 | dynamin 3 |
| miR 221/222 | MYLIP | Hs.484738 | myosin regulatory light chain interacting protein |
| miR 221/222 | MAT2A | Hs.516157 | methionine adenosyltransferase II, alpha |
| miR 221/222 | NFYB | Hs.84928 | nuclear transcription factor Y, beta |
| miR 221/222 | TAF9B | Hs.592248 | TAF9B RNA polymerase II, TATA box binding |
| | | | protein (TBP)-associated factor |

* Targets predicted from rat database.

The effect of an miRNA on the target genes can be used to identify agents that modulate the activity and/or expression of the miRNA.

Additional embodiments of the current invention provide a method of identifying an agent as an activator or inhibitor of an miRNA, wherein the miRNA belongs to a profile of miRNAs differentially expressed in a lymphatic vessel cell under a proinflammatory stimulus, the method comprising:

a) culturing a first lymphatic vessel cell in the presence of the miRNA and in the absence of the agent,

b) culturing a second lymphatic vessel cell in the presence of the miRNA and in the presence of the agent,

c) determining the expression and/or activity of a target gene in the first and the second lymphatic vessel cell,

d) comparing the expression and/or activity of the target gene in the first and the second lymphatic vessel cell, and

e) identifying the agent as the inhibitor or the activator of the miRNA, wherein the inhibitor of the miRNA negates the effect of the miRNA on the expression and/or activity of the target gene and the activator of miRNA enhances the effect of the miRNA on the expression and/or activity of target gene.

The agent can be a small molecule compound, miRNA antagomir, miRNA mimic, or miRNA itself. The target gene can be one or more of ZEB1, ZEB2, BCL2, ERB3, VEGF, PTEN, NF-κB1, E-CAD, STAT3, PDCD4, SPROUTY2, SEMA6, EPH2, EPHB4, E2F1, TIMP1, MAPK9, SOCS1, RNF11, p70S6K1, CUL2, FGF2, NRF2, SIRT1, Notch1, and PIK3CD.

In an embodiment of identifying an agent as an activator or inhibitor of an miRNA, the miRNA is miR-9 and the target genes are one or more of NF-κB, 13-Catenin, e-NOS, VE-Cadherin, and VEGFR3.

### Materials and Methods

Cell Culture and TNF Alpha Treatments

Rat lymphatic endothelial cells (RLECs) were isolated from rat mesenteric explants and their phenotype was verified by Prox 1, VEGFR3 and other lymphatic endothelial specific markers as described earlier (Hayes, Kossmann et al. 2003). Human lymphatic endothelial cells (HLEC) were purchased from Lonza (Basel, Switzerland). Cultures were grown in EGM2.MV media (Lonza) as described earlier (Dellinger and Brekken 2011). The endothelial cell cultures were grown to confluence and then maintained in low serum (1%) media prior to treatment. The cells were either treated with TNF-α (20 ng/ml) for 2 hrs, 24 hrs and 96 hrs or maintained for corresponding time points without treatment. The LECs were between passages 3-6 at the time of the experiments. TNF-α was purchased from R&D Systems, Inc. (Minneapolis, Minn.).

MicroRNA and Total RNA Preparation

Enriched Small RNA fractions were extracted from the LECs using miRNeasy and minElute kits (Qiagen, Valencia, Calif.). Quality and quantity of RNA were determined using a NanoDrop ND-1000 spectrophotometer (NanoDrop Technologies, Inc., Wilmington, Del.) and an Agilent Bioanalyzer system (Agilent Technologies, Inc., Santa Clara, Calif.).

MicroRNA Expression Profiling by Real-Time PCR Arrays

100 ng of miRNAs were reverse-transcribed using a specific RT2 miRNA First Strand cDNA Synthesis Kit (SABiosciences, Frederick, Md.). The cDNA was mixed with RT2 SYBR Green/ROX qPCR Master Mix and the mixture was added into a 384-well RT2 miRNA PCR Array (SABiosciences) that included pre-defined primer pairs for a set of 88 human miRNAs involved in regulation of immunity and inflammatory responses, and a panel of 8 housekeeping genes and controls. Experiments were performed in triplicate using biological replicates. RT2-PCR array was performed using an ABI Prism 7900 HT sequence detection system (Applied Biosystems, Foster City, Calif.) as per manufacturers' instructions. The threshold value was kept constant across arrays. Gene profiling and data analysis of miRNA expression was performed using web-based data analysis software (SABiosciences, Frederick, Md.). For normalization, miRNA expressions were compared between the treatment group at each time point and the control group. The ΔΔCt method was utilized to calculate the fold change. As normalization of the miRNA data is critical to eliminate the bulk of false positives the most stably expressed genes across the arrays were used to normalize the expression data including 3 of the manufacturer-provided housekeeping genes and miR152 whose expression was found to be unchanged across the arrays and time points determined by Normfinder (Chen, Wang et al. 2008; Hu, Dong et al. 2012).

Analysis and Target Prediction of microRNA

Those miRNA that showed more than 1.8-fold difference in fold change or had a significant p value, p<0.05 when compared with their corresponding controls were identified as differentially expressed. The list of differentially expressed genes were compared to various databases that predict targets for microRNAs: MirWalk (Dweep, H. et al., 2011, TargetScan (see Worldwide Website: targetscan.org), MIRANDA (see Worldwide Website: ebi.ac.uk), and PicTar-Vert (see Worldwide Website: pictar.mdc-berlin.de/).

miRNA Mimic and Inhibitor Transfection

HDLECs were grown to about 70% confluence and then transfected with 100-500 nM of miR-9 mimic or inhibitor (Life Technologies, Carlsbad, Calif.) using lipofectamine 2000 (Invitrogen, Gaithersburg, Md.) for 24-48 hrs as per manufacturer's instructions. For controls, cells were mock treated or transfected with control mimic or inhibitor sequences. Transfections were performed using OptiMem media (Invitrogen, Gaithersburg, Md.). The cells were grown to 70% confluence and then transfected in 500 μl antibiotic-free medium. The transfection medium was replaced after 6 hrs and the cells were then allowed to recover and were maintained in complete EGM2.MV medium (Lonza). Cells were closely monitored for cell death or toxicity.

LEC Tube Formation Assay

The ability of miR-9 mimic and miR-9 inhibitor-transfected LECs to form capillary networks was evaluated by endothelial tube formation assay in the absence or presence of TNF-α (20 ng/ml) as described earlier with some modifications (Luo, Zhou et al. 2011). 96-well plates were pre-coated with 40p Matrigel per well and allowed to polymerize for 1 hr at 370° C. LECs were trypsinized 48 hr post-transfection and 2×10⁴ cells were seeded into each well in 250 μl EGM2.MV (Lonza). The cells were allowed to form networks for about 16-24 hrs. In order to fluorescently visualize the cells, they were incubated with Calcein-AM (2 mM) (Invitrogen) for 30 mins at 370° C. As the fluorescent dye Calcein AM easily permeates live intact cells, this allows easier visualization of tube formation. Scrambled miRNA transfected cells were used as a control. Images were acquired by an inverted Olympus fluorescence microscope (Olympus). Quantitative analysis of network structure was performed with NIH-ImageJ software (see Worldwide Website: rsbweb.nih.gov/ij/) by counting the number of intersections in the network and measuring the total length of the structures. Results were plotted as mean±SEM.

Western Blots and Immunofluorescence

Western blot analysis was carried out as previously described (Chakraborty et al., 2011). Briefly, LECs were directly lyzed by 1×SDS buffer and proteins were separated on a 4-20% SDS-PAGE. Proteins were transferred into a nylon membrane and then probed with corresponding primary antibodies. The antibodies used were p-AKT (1:1000), total AKT (1:1000), VE-Cadherin (1:1000), N-Cadherin (1:1000), p-IκB (1:2000), total p-IκB (1:1000), p-NF-κB (1:1000), β-Catenin (1:1000), VEGFR3 (1:1000); eNOS (1:1000) and p-eNOS (1:1000). The blots were then probed with the corresponding secondary antibodies and developed with West Dura Extended duration Substrate. For loading control, we probed the blots with β-actin (1:1000) antibody.

Densitometry analyses on the resulting bands were performed using Quantity One Multi-Analyst Software (BioRad). For quantification experiments were repeated for 3 or 4 times for each sample and the resulting mean±SEM was calculated.

Immunofluorescence experiments were carried out using cultured LECs. Briefly, the HDLECs transfected with miR-9 mimics or inhibitors or control miRNA sequences were plated onto coverslips and grown to about 70% confluence. Cells were then fixed with 2% paraformaldehyde, permeabilized with ice-cold methanol and subjected to different primary antibodies for 1 hr. Normal mouse or rabbit serum was used in place of corresponding primary antibody as a control. After incubation with secondary antibody (conjugated to a fluorescent dye) for 1 hr in the dark followed by several stringent washes, the coverslips were mounted onto glass slides and allowed to partially air dry. Coverslips were mounted using Prolong antifade solution and allowed to cure overnight. The secondary antibody used in these experiments was Goat anti-Rabbit OG488. Maximum projections of series sections with step size of 0.5 micron thickness were imaged using the Leica AOBS SP2 Confocal microscope with an N-PLAN 20× dry objective of NA 1.15.

Statistical Analysis

Data analysis of miRNA expression across the miRNA arrays was performed using SABiosciences Online PCR Array Data Analysis Web Portal (see Worldwide Website: pcrdataanalysis.sabiosciences.com/pcr/arrayanalysis.php). All data are expressed as mean±SEM. Statistical analyses were done using a Student's t-test or one-way ANOVA as was appropriate. p<0.05 was regarded as statistically significant.

All patents, patent applications, provisional applications, and publications referred to or cited herein are incorporated by reference in their entirety, including all figures and tables, to the extent that they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—TNF-α Mediates Differential and Temporal Expression of miRNAs Associated with the Inflammatory Response in Lymphatic Endothelium

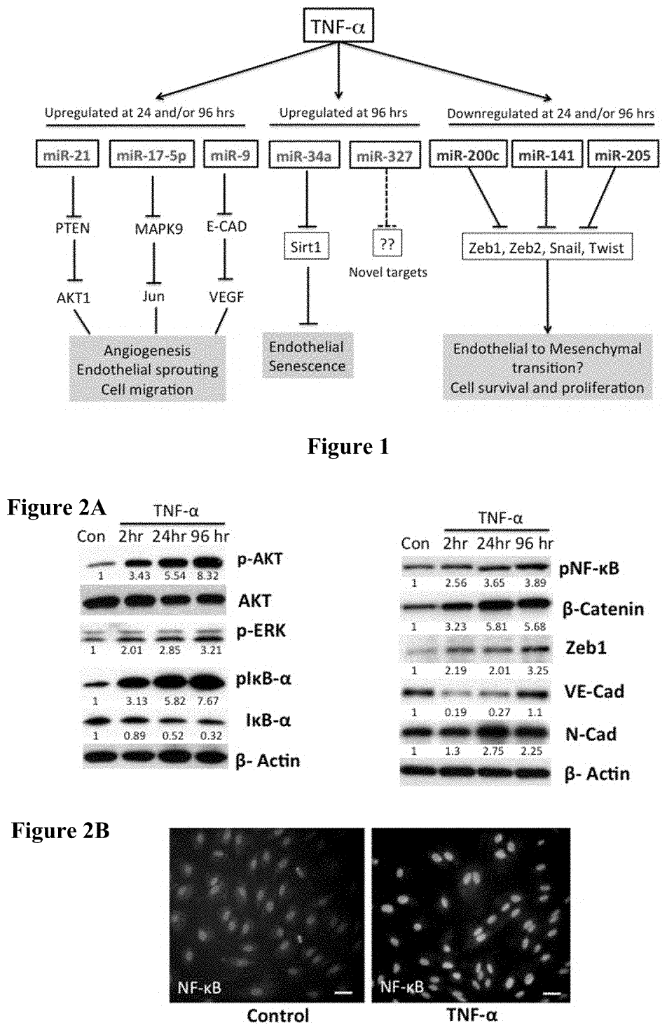

We analyzed the expression patterns of 88 different miRNAs in LECs after stimulation with TNF-α for 2 hr, 24 hr or 96 hr by using an Inflammatory and Autoimmune Response Real-Time-RT/PCR miRNA array. Upon quantification, out of 88 miRNAs, overall only 30 miRNAs showed a 1.8-fold difference or higher and/or had a significant p value (p<0.05) over the different time points analyzed (Table 2). Of these, only 1 miRNA was up-regulated at 2 hrs of TNF-α treatment while 3 miRNAs were down-regulated. At 24 hrs, the number of induced miRNAs increased to 11, whereas 3 miRNAs were down-regulated. Several miRNAs showed differential expression at 96 hr, which had a total of 12 up-regulated and 7 down-regulated miRNAs (Table 2). We found several overlapping miRNAs at each of the time points analyzed whereas a number of them were unique to a specific time point. Of these miR-136 was down-regulated at both 24 hr and 96 hrs while miR-20a, miR-203, miR-20b-5p, miR-21, miR-325-3p, miR-9 and miR-19a were up-regulated at both the time points. The miRNA target analysis described in the Methods section shows that the identified miRNAs' target genes could be broadly classified as involved in angiogenesis, inflammation, EMT/EndMT, cell proliferation and cellular senescence (Table 3 and FIG. 1). In addition, no information was available for miR-878, miR-327, miR-760-5p, miR-325-3p and miR-448 with respect to their roles in endothelial cell function.

miR-9 has been shown to promote endothelial cell motility and angiogenesis in HUVECs as well as to repress NF-κB1 in response to inflammatory stimuli (Bazzoni, Rossato et al. 2009; Zhuang, Wu et al. 2012). miR-9 was found to be induced in LECs by TNF-α at both 24 hr and 96 hr. Hence miR-9 was selected for further functional analysis as it potentially plays an important role in fine-tuning the TNF-α induced inflammatory reaction in LECs and plays a role in lymphangiogenesis.

TABLE 2
miRNAs in inflamed LECs at various time points

| miRNA | (2 hr) | p-Value | miRNA | 24(hr) | p-Value | miRNA | (96 hr) | p-Value |
|---|---|---|---|---|---|---|---|---|
| miR-144 | −1.98 | 0.0208 | miR-760-5p | −1.98 | 0.1438 | miR-291a-3p | −1.84 | 0.5236 |
| miR-20a | −3.41 | 0.9809 | miR-136 | −3.41 | 0.0810 | miR-327 | −1.99 | 0.7706 |
| miR-448 | −2.56 | 0.8966 | miR-141 | −2.56 | 0.2903 | miR-495 | −1.91 | 0.4155 |
| miR-200c | 1.98 | 0.7734 | miR-17-5p | 1.98 | 0.0260 | miR-136 | −3.47 | 0.1238 |
| | | | miR-203 | 2.8 | 0.0161 | miR-144 | −2.06 | 0.3750 |
| | | | miR-20a | 2.03 | 0.0188 | miR-145 | −2.19 | 0.4110 |
| | | | miR-20b-5p | 2.38 | 0.0248 | miR-205 | −2.20 | 0.0008 |
| | | | miR-21 | 1.95 | 0.0070 | miR-203 | 2.08 | 0.1141 |
| | | | miR-325-3p | 2.79 | 0.0113 | miR-34a | 2.27 | 0.0044 |
| | | | miR-9 | 1.89 | 0.0240 | miR-20a | 1.80 | 0.3234 |
| | | | miR-27a | 1.35 | 0.0400 | miR-20b-5p | 1.80 | 0.3193 |
| | | | miR-322 | 1.63 | 0.0129 | miR-21 | 1.81 | 0.4062 |
| | | | miR-878 | 1.89 | 0.1755 | miR-325-3p | 2.49 | 0.1674 |
| | | | miR-19a | 1.87 | 0.1256 | miR-497 | 3.19 | 0.0803 |
| | | | | | | miR-9 | 2.04 | 0.1287 |
| | | | | | | miR-34c | 1.79 | 0.2294 |
| | | | | | | miR-384-5p | 2.69 | 0.1393 |
| | | | | | | miR-19a | 1.86 | 0.4900 |
| | | | | | | miR-19b | 1.91 | 0.4372 |

TABLE 3
Pathways activated by the differentially expressed miRNAs and validated targets

| | Time point | Involvement in Pathways | Specific Validated |
|---|---|---|---|
| miRNA | expressed | (Relevant to ECs/LECs) | targets |
| miR-200c | 2 hr | EMT/EndMT, Apoptosis, | ZEB1, ZEB2 |
| | | Senescence | |
| miR-136 | 24 hr, 96 hr | EMT/EndMT, Apotosis | BCL2 |
| miR-205 | 96 hr | EMT/EndMT | ZEB1/2, ERB3, VEGFA |
| miR-141 | 24 hr | EMT/EndMT, Cell | PTEN, ZEB1, ZEB2 |
| | | proliferation, migration | |
| miR-9 | 24 hr, 96 hr | EMT/EndMT, Inflammation, | NF-KB1, E-CAD, |
| | | Angiogenesis | STAT3 |
| miR-21 | 24 hr, 96 hr | EMT/EndMT, Angiogenesis | PTEN, PDCD4 |
| miR-27a | 24 hr | Angiogenesis | SPROUTY2, SEMA6 |
| miR-20a | 2 hr, 24 hr, 96 hr | Angiogenesis | EPH2, EPHB4, VEGF |
| miR-20b-5p | 24 hr, 96 hr | Angiogenesis | VEGF |
| miR-17-5p | 24 hr | Angiogenesis, Cell | E2F1, TIMP1, MAPK9 |
| | | proliferation, Apoptosis | |
| miR-19b | 96 hr | Angiogenesis, Inflammation | SOCS1, RNF11 |
| miR-145 | 96 hr | Proliferation, differentiation, | p70S6K1, VEGF |
| | | angiogenesis and Apoptosis | |
| miR-322 | 24 hr | Angiogenesis, Oxidative | CUL2, FGF2 |
| | | Stress | |
| miR-144 | 2 hr, 96 hr | Oxidative Stress Response | NRF2 |
| miR-34a | 96 hr | Cellular senescence, | SIRT1 |
| | | proliferation | |
| miR-34c | 96 hr | Cellular senescence | Notch1, BCL2 |
| miR-497 | 96 hr | Apotosis | BCL2 |
| miR-384-5p | 96 hr | Apoptosis | PIK3CD |
| miR-291a-3p | 96 hr | Embryonic stem cell cycle | Unknown |
| | | regulator | |
| miR 397 | 96 hr | Unknown | Unknown |
| miR-325-3p | 24 hr, 96 hr | Unknown | Unknown |
| miR-327 | 96 hr | Unknown | Unknown |
| miR-760-5p | 24 hr | Unknown | Unknown |
| miR 448 | 2 hr | Unknown | Unknown |
| miR-878 | 24 hr | Unknown | Unknown |

### Example 2—TNF-α Promotes an Inflammatory Pathway in the Lymphatics and Also Increases Expression of Molecular Markers Associated with EMT/EndMT

We evaluated the role of TNF-α in mediating inflammation in the LECs. As shown in FIG. 2, TNF-α increased the phosphorylated levels of IκB at 2 hr, which was accompanied by a corresponding decrease in the levels of total IκB. Phospho NF-κB is also increased in the TNF-α treated cells and immunofluorescence data show that there is a marked nuclear translocation of NF-κB in LECs. TNF-α also modulated the activation of p-AKT and p-ERK in a time-dependent manner with maximum activation at 96 hr post treatment (FIG. 2). In addition, TNF-α also increased expression of Zeb1, β-Catenin and N-Cad, while decreasing levels of VE-Cad. The levels of the housekeeping control β-actin showed no change in expression (FIG. 2).

### Example 3—while miR-9 Inhibits NF-κB Signaling in LECs, it Promotes Lymphatic Tube Formation and Lymphangiogenesis Pathway as Well as EndMT

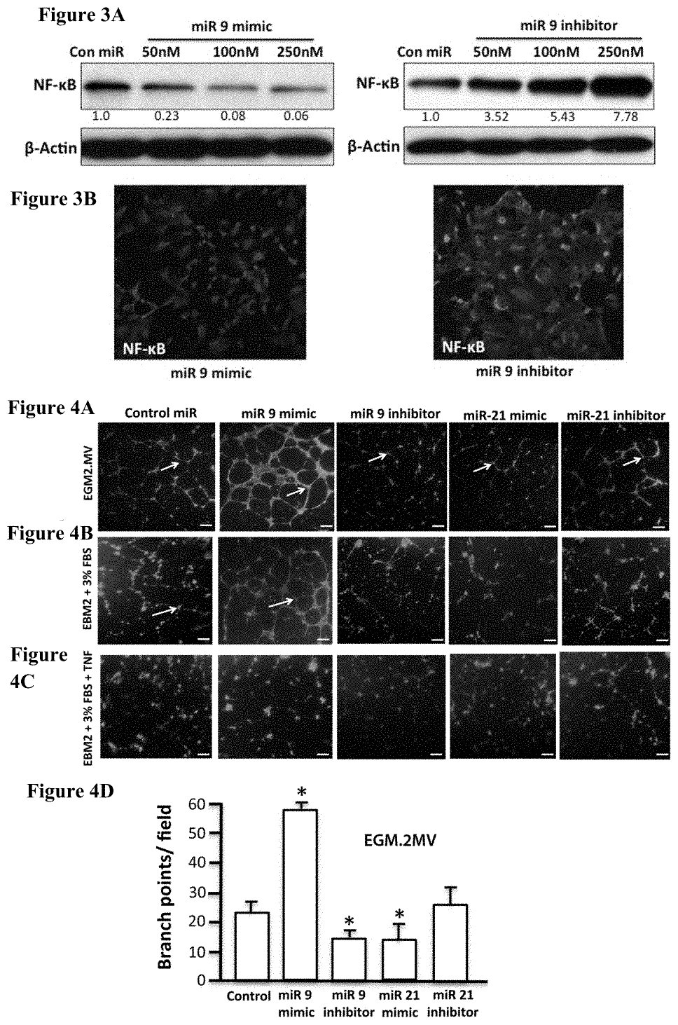

Since NF-κB has been previously shown to be a target for miR-9 (Bazzoni et al. 2009) and miRNA target analysis databases also showed a conserved miR-9 3′ UTR binding seed sequence in the NF-κB gene, we checked the NF-κB protein levels in LECs transfected with increasing concentrations of miR-9 mimic and miR-9 inhibitor. miR-9 overexpression significantly inhibited NF-κB in a concentration-dependent manner, while inhibition of endogenous miR-9 increased the relative levels of NF-κB (FIG. 3).

Though several studies have shown the close association of inflammation and lymphangiogenesis (Ji 2007; Pober and Sessa 2007; Podgrabinska, Kamalu et al. 2009; Vigl, Aebischer et al. 2011), the role of the proinflammatory cytokine TNF-α in modulating lymphangiogenesis remains controversial (Polzer, Baeten et al. 2008; Baluk, Yao et al. 2009; Chaitanya, Franks et al. 2010; Jones, Li et al. 2012). Since miR-9 was up-regulated in the TNF-α treated LECs and it also directly targeted NF-κB, we assessed the effects of miR-9 in LECs on lymphangiogenesis in the absence or presence of TNF-α. LECs transfected with either a control miRNA oligonucleotide or miR-9 mimic or inhibitor were subjected to matrigel assay as described in the Methods section. Compared to the control miR, overexpression of miR-9 in LECs led to a significant increase in the ability of LECs to form tube-like networks with uninterrupted branch points (FIGS. 4A and 4B). miR-9 inhibitor significantly reduced tube formation.

LEC tube formation assays were also carried out by modulating the levels of miR-21, another miRNA that showed an increase in the TNF-α treated LECs in our analysis. miR-21 mimics caused a significant reduction in LEC tube formation, whereas the miR-21 antagomirs showed an increase compared to the mimic, although not significant. As shown in FIG. 4, TNF-α did not promote tube formation and miR-9 or miR-21 treatment did not reverse the effect of TNF-α on LEC as evident from the decreased branch lengths and nodes.

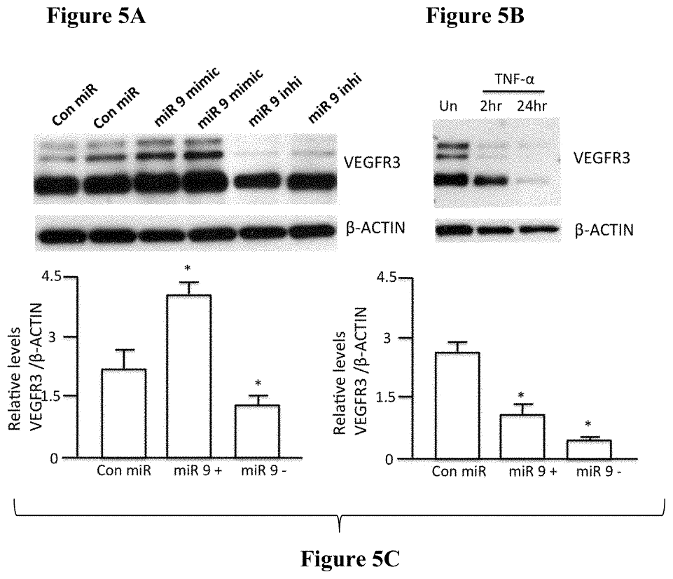

Since vascular endothelial growth factor (VEGF) receptor 3 (VEGFR3) is the main determinant of lymphangiogenesis and has been shown to be important for growth and survival signals in LECs, we then determined the effects of miR-9 on VEGFR3 expression. Overexpression of miR-9 significantly increased the expression of VEGFR3 in LECs, whereas miR-9 inhibitors decreased the relative levels of VEGFR3 (FIG. 5).

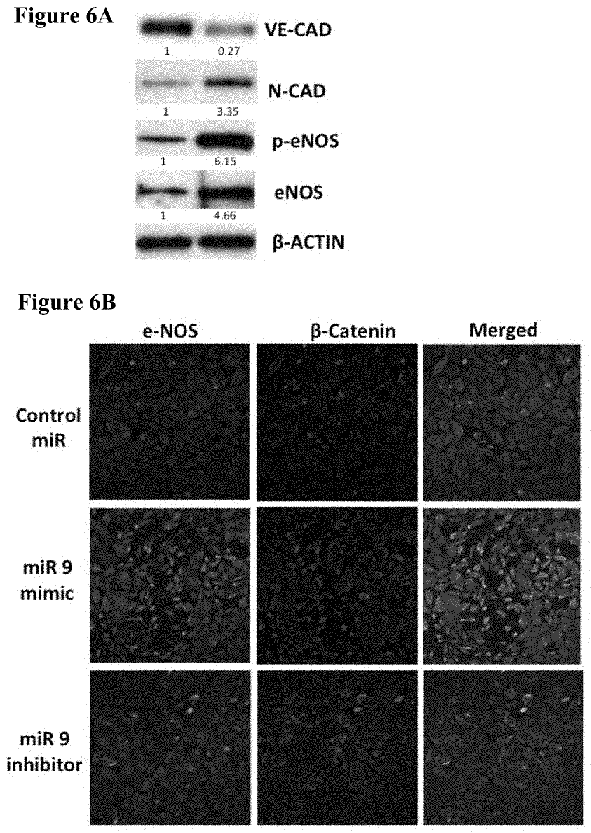

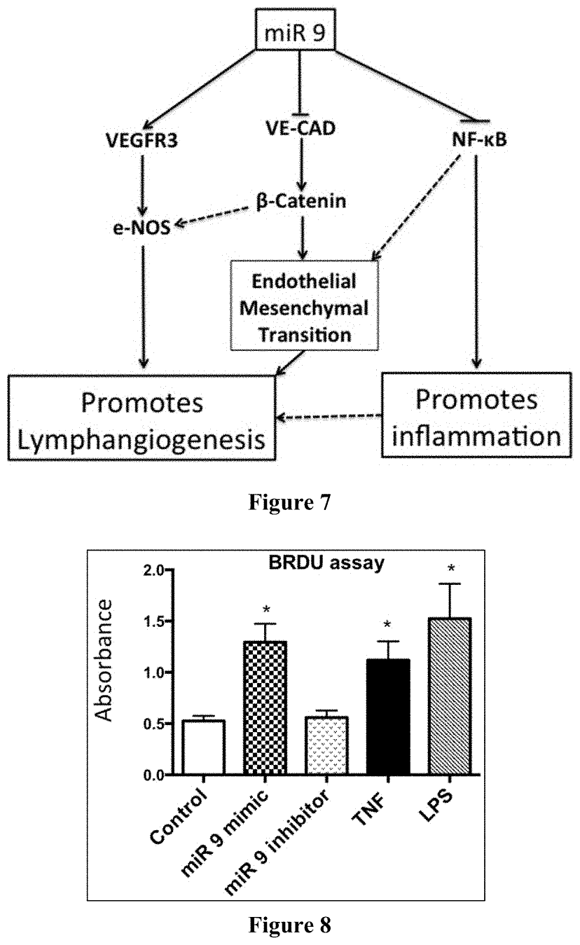

TNF-α decreased VEGFR3 expression in a time-dependent manner with significant decrease by 2 hr and almost 75% by 24 hrs. To further delineate the molecular mechanisms regulating miR-9 mediated increase of VEGFR3 expression and subsequent LEC tube formation we investigated the effects of miR-9 on the expression patterns of VE-Cadherin, 3-Catenin, e-NOS and p-eNOS. These molecules have been reported in various studies to regulate VEGFR3 expression and/or lymphangiogenesis as well as being implicated in EMT or EndMT (Lahdenranta, Hagendoorn et al. 2009; Lohela, Bry et al. 2009; Ma, Young et al. 2010). As shown in FIG. 6, miR-9 markedly decreased the expression of VE-Cadherin while increasing the levels of N-Cadherin. Also, miR-9 mimics increased the levels of e-NOS and p-eNOS protein levels in LECs. Immunofluorescence analysis of LECs showed an increased migration of β-catenin from the cell junctions into the cytoplasm and nucleus in LECs overexpressing miR-9, and an increased eNOS expression in the mimic-transfected cells when compared to the control miR- and the miR-9 inhibitor (FIG. 6).

### Example 4—Methods of Treating Inflammation-Mediated Lymphatic Diseases Using miR-9 Agonists

miR-9 mimics induce tube formation; thus miR-9 expression can be increased in pathologies that would benefit from tube formation and inhibition of inflammation, such as lymphedema and IBO. In one embodiment of the present invention, the method of treating an inflammation-mediated lymphatic disease comprises administering to a subject in need thereof a pharmaceutically effective amount of an agonist of miR-9. The agonist of miR-9 can be a polynucleotide comprising pri-miRNA, pre-miRNA or a mature miRNA sequence of miR-9. In another embodiment, the agonist can be an expression vector capable of expressing miR-9 in the subject.

### Example 5—Methods of Treating Inflammation-Mediated Lymphatic Diseases Using miR-9 Antagonists

Treatment of certain inflammation-mediated lymphatic diseases, for example, cancer metastasis, may comprise reducing lymphatic tube formation. Certain embodiments of the present invention provide a method of treating an inflammation-mediated lymphatic disease, the method comprising administering to a subject in need thereof a pharmaceutically effective amount of an antagonist of miR-9. The antagonist of miR-9 can be a polynucleotide capable of hybridizing with pri-miR-9, pre-miR-9 or mature miR-9 via a sequence which is complementary or substantially complementary to the sequence of miR-9. Typically, a sequence which is about 70%, about 75%, about 80%, about 85%, about 90%, about 95%, or about 100% complementary to target sequence is capable of hybridizing with the target sequence. In another embodiment, the antagonist of miR-9 can be an expression vector capable of expressing a polynucleotide capable of hybridizing with pri-miR-9, pre-miR-9 or mature miR-9 via a sequence which is complementary or substantially complementary to the sequence of miR-9.

### Example 6—Profiles of miRNAs Differentially Expressed in LECs Exposed to a Proinflammatory Stimulus

Differential expressions of miRNAs are found to broadly be involved in regulation of pathways underlying inflammation (miR-9, miR-21), angiogenesis (miR-20a, miR-20b-5p, miR-21, miR-9, miR-145, miR-27a, miR-17-5p, miR-322, miR-19b), EMT/EndMT (miR-141, miR-200c, miR-136, miR-21, miR-9), cellular senescence (miR-34a, miR-34c) and cell proliferation (miR-203, miR-141, miR-17-5p). Furthermore, the target analyses indicate a group of miRNAs (miR-291a-3p, miR-397, miR-325-3p, miR-327, miR-760-5p, miR-448 and miR-878) have no documented role in endothelial biology and some of them have no validated target, suggesting that these miRNAs could have novel roles in the regulation of lymphatic endothelial functions.

While miR-9 decreases inflammation, it augments the lymphangiogenesis and EndMT pathways in LECs. Thus, our data has provided the first evidence of a set of regulatory miRNAs involved in different cellular pathways that may potentially modulate inflammation and lymphangiogenic signaling in the lymphatics. Here we have discussed how the miRNAs identified in this study correlate to various signaling mechanisms, specifically to cell proliferation, angiogenesis, and endothelial to mesenchymal transition (EndMT), which could be related to the responses of LECs under inflammatory stimuli.

miR-21 and miR-17-92 Cluster

We found a fairly large group of differentially expressed miRNAs that have been previously shown to be closely associated with regulation of angiogenesis. Several recent studies have provided detailed insights into how miRNAs regulate angiogenesis (Suarez and Sessa 2009; Toffanin, Sia et al. 2012) but only one miRNA to date has been implicated in the process of lymphangiogenesis (Jones, Li et al. 2012). One of the most well-studied miRNAs in endothelial inflammation and dysfunction, as well as angiogenesis, is miR-21, which is significantly up-regulated in LECs after prolonged exposure to inflammatory stimuli (Table 2). miR-21 regulates cell proliferation by suppressing PTEN, a potent negative regulator of the PI3K/Akt signaling pathway (Meng, Henson et al. 2007). Previous studies have shown that PTEN suppresses Akt signaling, which in turn decreases eNOS activity and VCAM-1 expression in vascular endothelial cells stimulated by TNF-α (Tsoyi, Jang et al. 2010). These findings suggest that miR-21 promotes inflammation in endothelial cells. However, miR-21 has a very complex relationship with NF-κB signaling as it enhances NF-κB through AKT activation, whereas other studies have shown miR-21 is a trans-activation target of NF-κB (Iliopoulos, Jaeger et al. 2010; Young, Santhanam et al. 2010). The scenario is no less complicated with respect to angiogenesis as miR-21 has been shown to induce tumor angiogenesis through activation of the AKT and ERK pathways (Liu, Li et a. 2011), while it displays an antiangiogenic role in vascular endothelial cells by reducing endothelial cell proliferation, migration and tube formation by targeting Rho B (Sabatel, Malvaux et al. 2011). The latter study is consistent with our finding that miR-21 mimics reduced tube formation in LECs (FIG. 4), suggesting that it possibly has an antilymphangiogenic role.

Several miRNAs identified in this study (miR-17-5p, miR-19a, miR-19b-1 and miR-20a) belong to the miR-17-92 cluster that comprises seven miRNAs, transcribed as a polycistronic unit and significantly amplified in B-cell lymphoid malignancies (Tanzer and Stadler 2004; Inomata, Tagawa et al. 2009). Members of this cluster have been shown to exhibit a cell-intrinsic antiangiogenic effect in endothelial cells as well as to regulate inflammation (Doebele, Bonauer et al. 2010; Philippe, Alsaleh et al. 2013). miR-17-5p has also been induced by TNF-α in HUVECs and is up-regulated in a number of inflammatory disorders (Suarez and Sessa 2009). miR-17-5p and miR-20a have been shown to be associated with cellular proliferation and apoptosis by targeting the E2F family of proteins (Cloonan, Brown et al. 2008). Furthermore, a miR-19 regulon has been shown to positively control NF-κB signaling by suppressing negative regulators of NF-κB, and thus targeting this miRNA, and linked family members could regulate the activity of NF-κB signaling in inflammation (Gantier, Stunden et a. 2012).

miR-141, miR-136, miR-205 and miR-200c in EndMT of LECs

Evidence from recent studies suggests that EndMT is an important contributor to cardiac and vascular development as well as to pathophysiological vascular remodeling and tissue remodeling (Arciniegas, Frid et al. 2007). EndMT bears close similarity to the aberrant cellular phenotypic switching or EMT that underlies a number of adult pathological conditions including fibrosis, wound repair, inflammation, and cancer metastasis and is characterized by loss of E-cadherin, 3-catenin relocalization, and acquisition of elongated cell shape (Arciniegas, Frid et al. 2007; Kovacic, Mercader et al. 2012). It has been recently demonstrated that lymphangiogenesis is an important feature in the progression of kidney fibrosis, although the exact molecular mechanisms mediating these processes are unclear. The number of lymphatic vessels has been shown to be increased in areas of fibrosis than inflammation. and has been shown to be closely correlated with severity of fibrosis and tissue injury (El-Chemaly, Malide et al. 2009; Sakamoto, Ito et al. 2009; Vass, Shrestha et al. 2012). Also, significantly, TGFβ (an established EndMT inducer and a key mediator for tissue fibrosis) stimulates VEGFC and lymphangiogenesis (Suzuki, Ito et al. 2012). It is notable that in this study we found a number of miRNAs that have been implicated either in EndMT or EMT to be differentially expressed in LECs in response to TNF-α (Table 3, FIG. 1).

Magenta et al. (Magenta, Cencioni et al. 2011) have shown that in response to oxidative stress miR-200c was the most highly expressed in vascular endothelial cells. miR-200c overexpression caused HUVECs' growth arrest, apoptosis and senescence, and was partially rescued by miR-200c inhibition. The pro-survival protein ZEB1 has been identified as a direct target of miR-200c and ZEB1 is a key molecule involved in the process of EMT and EndMT that directly suppresses E-Cadherin (Liu, El-Naggar et al. 2008; Magenta, Cencioni et al. 2011). miR-141 also directly targets ZEB1. However, Ulrike Burk et al. (Burk, Schubert et al. 2008) have shown that ZEB1 directly suppresses transcription of microRNA-200 family members miR-141 and miR-200c, and triggers an microRNA-mediated feed-forward loop that stabilizes EMT and promotes invasion. Down-regulation of miR-141 and miR-205 have been implicated in EMT progression, whereas up-regulation of miR-21 and miR-9 have been linked to EndMT and EMT, respectively (Kumarswamy, Volkmann et al. 2012; Lu, Huang et al. 2012). The expression patterns of miR-141, miR-205, miR-9 and miR-21 miRNAs that we observed in inflamed LECs are similar, as discussed above (Table 2, FIG. 1), suggesting that TNF-α would also induce EndMT in the LECs by activation and inhibition of specific miRNAs. Our data supports this as TNF-α induces ZEB1, decreases the expression levels of VE-Cadherin and increases N-Cadherin in a time-dependent manner (FIG. 2), which is characteristic of EMT/EndMT progression.

Role of miR-9 in Lymphangiogenesis and Inflammation

Among the different miRNAs induced by TNF-α in LEC, one that stood out in our screening, was miR-9, which has been previously linked to progression of tumor metastasis and angiogenesis (Ma, Young et al. 2010; Zhuang, Wu et al. 2012). Tumor secreted miR-9 is also involved in endothelial cell proliferation, migration and increased angiogenesis through activation of the JAK/STAT pathway (Ma, Young et al. 2010; Zhuang, Wu et al. 2012). Besides, it has also been shown that miR-9 targets E-Cadherin to promote cancer metastasis through an EMT-like mechanism (Ma, Young et al. 2010; Zhuang, Wu et al. 2012). Thus, as this miRNA seemed to have an important role in angiogenesis, EMT and inflammation, we focused on its functional role in the lymphatics.

TNF-α and LPS have been shown to up-regulate miR-9 in monocytes and neutrophils, and monoclonal antibodies against TNF-α were found to completely abrogate miR-9 expression in these cells (Bazzoni, Rossato et al. 2009). We found that TNF-α significantly increased miR-9 expression in LECs at both 24 hr and 96 hr. Furthermore TNF-α activates the expression of both p-I-κB and p-NF-κB, and also causes nuclear translocation of NF-κB (FIG. 2), thereby initiating the onset of inflammatory signaling in the lymphatics. It has been proposed that as NF-κB is a key regulator of inflammation, the NF-κB levels are likely to be a strictly controlled and timely regulated event for the proper progression of the inflammatory response, and several miRNAs have been identified that activate or inhibit NF-κB expression (Bazzoni, Rossato et al. 2009; Ma, Becker Buscaglia et al. 2011; Sun, Icli et al. 2012). We identify a TNF-α induced miRNA, miR-9, as a negative regulator of NF-κB in LECs (FIG. 3), indicating a potential feedback regulation. Thus, it is conceivable that the same inflammatory stimuli activate miRNA with opposing functions to maintain the balance between inflammation progression and resolution. This is supported by the data presented in this study, as TNF-α also induces miR-19 in LECs, an miRNA that is known to positively regulate NF-κB (Gantier, Stunden et a. 2012).

The VEGF family VEGF-C, VEGF-A, and VEGF-D have also been significantly implicated in inflammatory lymphangiogenesis (Kubo, Cao et al. 2002; Cursiefen, Chen et al. 2004; Baluk, Tammela et al. 2005; Watari, Nakao et al. 2008; Kim, Koh et al. 2009). Inflammatory signals have been shown to induce VEGF-C expression (Ristimaki, Narko et al. 1998). Activation of the NF-κB pathway in LECs up-regulates Prox1 and VEGFR-3, increasing the sensitivity of pre-existing lymphatic vessels to VEGF-C and VEGF-D produced by leukocytes and promoting lymphangiogenesis (Flister, Wilber et al. 2010). However, in contrast, during acute skin inflammation in mice, VEGFR-3 mRNA and protein is significantly decreased in inflamed lymphatics. This is consistent with the presented data that TNF-α decreased VEGFR3 expression and tube formation in LECs (FIGS. 4 and 5). Inflammatory cytokines IL-1β has also been shown to inhibit lymphatic tube formation in vitro by induction of miR-1236, which negatively targets VEGFR3 (Jones, Li et al. 2012). Although several studies have shown that TNF-α does not promote LEC tube formation and also suppresses lymphangiogenesis in murine and human arthritic joints (Polzer, Baeten et al. 2008; Chaitanya, Franks et al. 2010; Jones, Li et al. 2012), conflicting evidence about its in vivo roles exists (Baluk, Yao et al. 2009). It is believed that inflammatory cytokines such as IL-1β and possibly TNF-α contribute to initial lymphangiogenesis, in part, by inducing expression of VEGFs and adhesion molecules such as ICAM-1, which in turn recruits inflammatory cells that express lymphangiogenic factors (Baluk, Yao et al. 2009; Jones, Li et al. 2012).

VEGFR3 has been shown to induce e-NOS, a major lymphangiogenic molecule (Lahdenranta, Hagendoorn et al. 2009; Coso, Zeng et al. 2012). This would also explain why even though TNF-α seems to promote EndMT like phenomenon in LECs, it does not promote LEC tube formation. It is interesting that miR-9 mimics significantly up-regulated expression of VEGFR3 as well as also induced e-NOS and p-eNOS levels in LECs (FIG. 6). We thus propose that miR-9, which is induced by TNF-α, maintains this balance of inflammatory lymphangiogenesis as it increases VEGFR3 and eNOS expression (FIGS. 5 and 6) and induces LEC tube formation, while inhibiting NF-κB mediated regulation of the inflammatory process (FIGS. 3 and 4). It is also important to note that miR-9 or miR-21 treatment did not reverse the effect of TNF-α on LEC.

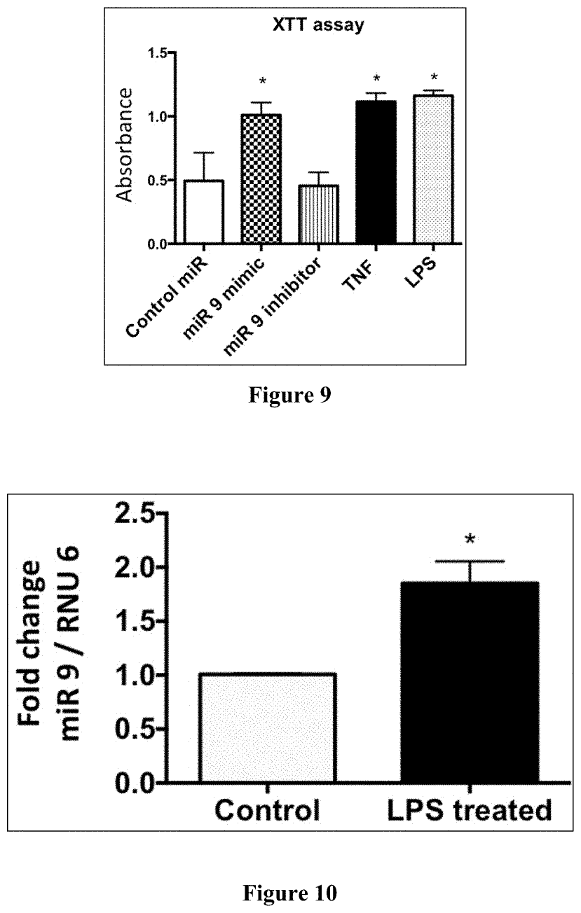

The data presented in FIGS. 8 and 9 show that miR-9 is involved in lymphatic endothelial cell (LEC) proliferation, which is one of the key phenotypes in the lymphangiogenesis process. Thus these data support our idea that miR-9 is involved in the lymphangiogenesis process. In addition, FIG. 10 provides evidence that, under inflammatory conditions in an in vivo model, miR-9 is up regulated, correlating with in vitro cell culture data.

This can be explained by our own findings that TNF-α does not promote LEC tube formation and possibly does so by inducing a set of miRNA with pro-lymphangiogenic and anti-lymphangiogenic functions (Table 3). Taken together, our results demonstrate for the first time that inflamed LECs express a specific profile of miRNAs that regulate several critical pathways underlying inflammation, angiogenesis, EMT/EndMT, viability, cell proliferation, and cellular senescence.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

1. Adams, R. H. and K. Alitalo (2007). “Molecular regulation of angiogenesis and lymphangiogenesis.” Nat Rev Mol Cell Biol 8(6): 464-478.
2. Alexander, J. S., G. V. Chaitanya, et al. (2010). “Emerging roles of lymphatics in inflammatory bowel disease.” Annals of the New York Academy of Sciences 1207 (Suppl 1): E75-85.
3. Arciniegas, E., M. G. Frid, et al. (2007). “Perspectives on endothelial-to-mesenchymal transition: potential contribution to vascular remodeling in chronic pulmonary hypertension.” Am J Physiol Lung Cell Mol Physiol 293(1): L1-8.
4. Baluk, P., T. Tammela, et al. (2005). “Pathogenesis of persistent lymphatic vessel hyperplasia in chronic airway inflammation.” The Journal of Clinical Investigation 115(2): 247-257.
5. Baluk, P., L. C. Yao, et al. (2009). “TNF-alpha drives remodeling of blood vessels and lymphatics in sustained airway inflammation in mice.” The Journal of Clinical Investigation 119(10): 2954-2964.
6. Bazzoni, F., M. Rossato, et al. (2009). “Induction and regulatory function of miR-9 in human monocytes and neutrophils exposed to proinflammatory signals.” Proceedings of the National Academy of Sciences of the United States of America 106(13): 5282-5287.
7. Bramsen et al. (2011). “Chemical Modification of Small Interfering RNA.” Methods in Molecular Biology, 721:77-103.
8. Burk, U., J. Schubert, et a. (2008). “A reciprocal repression between ZEB1 and members of the miR-200 family promotes EMT and invasion in cancer cells.” EMBO Rep 9(6): 582-589.
9. Chaitanya, G. V., S. E. Franks, et a. (2010). “Differential cytokine responses in human and mouse lymphatic endothelial cells to cytokines in vitro.” Lymphatic Research and Biology 8(3): 155-164.
10. Chen, S. Y., Y. Wang, et al. (2008). “The genomic analysis of erythrocyte microRNA expression in sickle cell diseases.” PLOS ONE 3(6): e2360.
11. Cloonan, N., M. K. Brown, et al. (2008). “The miR-17-5p microRNA is a key regulator of the GI/S phase cell cycle transition.” Genome Biology 9(8): R127.
12. Coso, S., Y. Zeng, et al. (2012). “Vascular endothelial growth factor receptor-3 directly interacts with phosphatidylinositol 3-kinase to regulate lymphangiogenesis.” PLOS ONE 7(6): e39558.
13. Cursiefen, C., L. Chen, et al. (2004). “VEGF-A stimulates lymphangiogenesis and hemangiogenesis in inflammatory neovascularization via macrophage recruitment.” The Journal of Clinical Investigation 113(7): 1040-1050.
14. Dellinger, M. T. and R. A. Brekken (2011). “Phosphorylation of Akt and ERK1/2 is required for VEGF-A/VEGFR2-induced proliferation and migration of lymphatic endothelium.” PLOS ONE 6(12): e28947.
15. Doebele, C., A. Bonauer, et al. (2010). “Members of the microRNA-17-92 cluster exhibit a cell-intrinsic antiangiogenic function in endothelial cells.” Blood 115(23): 4944-4950.
16. El-Chemaly, S., D. Malide, et al. (2009). “Abnormal lymphangiogenesis in idiopathic pulmonary fibrosis with insights into cellular and molecular mechanisms.” Proceedings of the National Academy of Sciences of the United States of America 106(10): 3958-3963.
17. Flister, M. J., A. Wilber, et al. (2010). “Inflammation induces lymphangiogenesis through up-regulation of VEGFR-3 mediated by NF-kappaB and Prox1.” Blood 115(2): 418-429.
18. Gantier, M. P., H. J. Stunden, et al. (2012). “A miR-9 regulon that controls NF-kappaB signaling.” Nucleic Acids Research 40(16): 8048-8058.
19. Hayes, H., E. Kossmann, et al. (2003). “Development and characterization of endothelial cells from rat microlymphatics.” Lymphatic Research and Biology 1(2): 101-119.
20. Hu, Z., J. Dong, et al. (2012). “Serum microRNA profiling and breast cancer risk: the use of miR-484/191 as endogenous controls.” Carcinogenesis 33(4): 828-834.
21. Huggenberger, R., S. S. Siddiqui, et al. (2011). “An important role of lymphatic vessel activation in limiting acute inflammation.” Blood 117(17): 4667-4678.
22. Iliopoulos, D., S. A. Jaeger, et al. (2010). “STAT3 activation of miR-21 and miR-181b-1 via PTEN and CYLD are part of the epigenetic switch linking inflammation to cancer.” Mol Cell 39(4): 493-506.
23. Inomata, M., H. Tagawa, et al. (2009). “MicroRNA-17-92 down-regulates expression of distinct targets in different B-cell lymphoma subtypes.” Blood 113(2): 396-402.
24. Ji, R. C. (2007). “Lymphatic endothelial cells, inflammatory lymphangiogenesis, and prospective players.” Current Medicinal Chemistry 14(22): 2359-2368.
25. Johnson, L. A., S. Clasper, et al. (2006). “An inflammation-induced mechanism for leukocyte transmigration across lymphatic vessel endothelium.” J Exp Med 203(12): 2763-2777.
26. Johnson, L. A. and D. G. Jackson (2010). “Inflammation-induced secretion of CCL21 inlymphatic endothelium is a key regulator of integrin-mediated dendritic cell transmigration.” Int Immunol 22(10): 839-849.
27. Jones, D., Y. Li, et al. (2012). “Mirtron microRNA-1236 inhibits VEGFR-3 signaling during inflammatory lymphangiogenesis.” Arteriosclerosis, Thrombosis, and Vascular Biology 32(3): 633-642.
28. Kazenwadel, J., M. Z. Michael, et al. (2010). “Prox1 expression is negatively regulated by miR-181 in endothelial cells.” Blood 116(13): 2395-2401.
29. Kim, H., R. P. Kataru, et al. (2012). “Regulation and implications of inflammatory lymphangiogenesis.” Trends in Immunology 33(7): 350-356.
30. Kim, K. E., Y. J. Koh, et al. (2009). “Role of CD11b+ macrophages in intraperitoneal lipopolysaccharide-induced aberrant lymphangiogenesis and lymphatic function in the diaphragm.” The American Journal of Pathology 175(4): 1733-1745.
31. Kim, V. N. (2005). “MicroRNA biogenesis: coordinated cropping and dicing.” Nat Rev Mol Cell Biol 6(5): 376-385.
32. Kovacic, J. C., N. Mercader, et al. (2012). “Epithelial-to-mesenchymal and endothelial-to-mesenchymal transition: from cardiovascular development to disease.” Circulation 125(14): 1795-1808.
33. Kubo, H., R. Cao, et al. (2002). “Blockade of vascular endothelial growth factor receptor-3 signaling inhibits fibroblast growth factor-2-induced lymphangiogenesis in mouse cornea.” Proceedings of the National Academy of Sciences of the United States of America 99(13): 8868-8873.
34. Kuehbacher, A., C. Urbich, et al. (2007). “Role of Dicer and Drosha for endothelial microRNA expression and angiogenesis.” Circulation Research 101(1): 59-68.
35. Kumarswamy, R., I. Volkmann, et al. (2012). “Transforming growth factor-beta-induced endothelial-to-mesenchymal transition is partly mediated by microRNA-21.” Arteriosclerosis, Thrombosis, and Vascular Biology 32(2): 361-369.
36. Lahdenranta, J., J. Hagendoorn, et al. (2009). “Endothelial nitric oxide synthase mediates lymphangiogenesis and lymphatic metastasis.” Cancer Research 69(7): 2801-2808.
37. Lawrence, T. (2009). “The nuclear factor NF-kappaB pathway in inflammation.” Cold Spring Harb Perspect Biol 1(6): a001651.
38. Liu, L. Z., C. Li, et a. (2011). “MiR-21 induced angiogenesis through AKT and ERK activation and HIF-1alpha expression.” PLOS ONE 6(4): e19139.
39. Liu, Y., S. El-Naggar, et al. (2008). “Zeb1 links epithelial-mesenchymal transition and cellular senescence.” Development 135(3): 579-588.
40. Lohela, M., M. Bry, et al. (2009). “VEGFs and receptors involved in angiogenesis versus lymphangiogenesis.” Current Opinion in Cell Biology 21(2): 154-165.
41. Lu, M. H., C. C. Huang, et al. (2012). “Prospero homeobox 1 promotes epithelial-mesenchymal transition in colon cancer cells by inhibiting E-cadherin via miR-9.” Clin Cancer Res 18(23): 6416-6425.
42. Luo, Y., H. Zhou, et alt (2011). “The fungicide ciclopirox inhibits lymphatic endothelial cell tube formation by suppressing VEGFR-3-mediated ERK signaling pathway.” Oncogene 30(18): 2098-2107.
43. Ma, L., J. Young, et al. (2010). “miR-9, a MYC/MYCN-activated microRNA, regulates E-cadherin and cancer metastasis.” Nature Cell Biology 12(3): 247-256.
44. Ma, X., L. E. Becker Buscaglia, et al. (2011). “MicroRNAs in NF-kappaB signaling.” Mol Cell Biol 3(3): 159-166.
45. Magenta, A., C. Cencioni, et al. (2011). “miR-200c is upregulated by oxidative stress and induces endothelial cell apoptosis and senescence via ZEB1 inhibition.” Cell Death Differ 18(10): 1628-1639.
46. Meng, F., R. Henson, et al. (2007). “MicroRNA-21 regulates expression of the PTEN tumor suppressor gene in human hepatocellular cancer.” Gastroenterology 133(2): 647-658.
47. O'Connell, R. M., D. S. Rao, et al. (2012). “microRNA regulation of inflammatory responses.” Annu Rev Immunol 30: 295-312.
48. Pedrioli, D. M., T. Karpanen, et al. (2010). “miR-31 functions as a negative regulator of lymphatic vascular lineage-specific differentiation in vitro and vascular development in vivo.” Molecular and cellular biology 30(14): 3620-3634.
49. Pegu, A., S. Qin, et al. (2008). “Human lymphatic endothelial cells express multiple functional TLRs.” Journal of Immunology 180(5): 3399-3405.
50. Philippe, L., G. Alsaleh, et al. (2013). “The miR-17 approximately 92 Cluster: A Key Player in the Control of Inflammation during Rheumatoid Arthritis.” Frontiers in Immunology 4: 70.
51. Pober, J. S. and W. C. Sessa (2007). “Evolving functions of endothelial cells in inflammation.” Nat Rev Immunol 7(10): 803-815.
52. Podgrabinska, S., O. Kamalu, et al. (2009). “Inflamed lymphatic endothelium suppresses dendritic cell maturation and function via Mac-1/ICAM-1-dependent mechanism.” Journal of Immunology 183(3): 1767-1779.
53. Polzer, K., D. Baeten, et al. (2008). “Tumour necrosis factor blockade increases lymphangiogenesis in murine and human arthritic joints.” Annals of the Rheumatic Diseases 67(11): 1610-1616.
54. Ran, S. and K. E. Montgomery (2012). “Macrophage-Mediated Lymphangiogenesis: The Emerging Role of Macrophages as Lymphatic Endothelial Progenitors.” Cancers (Basel) 4(3): 618-657.
55. Randolph, G. J., V. Angeli, et al. (2005). “Dendritic-cell trafficking to lymph nodes through lymphatic vessels.” Nat Rev Immunol 5(8): 617-628.
56. Ristimaki, A., K. Narko, et al. (1998). “Proinflammatory cytokines regulate expression of the lymphatic endothelial mitogen vascular endothelial growth factor-C.” J Biol Chem 273(14): 8413-8418.
57. Sabatel, C., L. Malvaux, et al. (2011). “MicroRNA-21 exhibits antiangiogenic function by targeting RhoB expression in endothelial cells.” PLOS ONE 6(2): e16979.
58. Sakamoto, I., Y. Ito, et al. (2009). “Lymphatic vessels develop during tubulointerstitial fibrosis.” Kidney Int 75(8): 828-838.
59. Sawa, Y., Y. Sugimoto, et al. (2007). “Effects of TNF-alpha on leukocyte adhesion molecule expressions in cultured human lymphatic endothelium.” J Histochem Cytochem 55(7): 721-733.
60. Sawa, Y. and E. Tsuruga (2008). “The expression of E-selectin and chemokines in the cultured human lymphatic endothelium with lipopolysaccharides.” J Anat 212(5): 654-663.
61. Suarez, Y., C. Fernandez-Hernando, et al. (2007). “Dicer dependent microRNAs regulate gene expression and functions in human endothelial cells.” Circulation Research 100(8): 1164-1173.
62. Suarez, Y. and W. C. Sessa (2009). “MicroRNAs as novel regulators of angiogenesis.” Circulation Research 104(4): 442-454.
63. Sun, X., B. Icli, et al. (2012). “MicroRNA-181b regulates NF-kappa B-mediated vascular inflammation.” The Journal of Clinical Investigation 122(6): 1973-1990.
64. Suzuki, Y., Y. Ito, et al. (2012). “Transforming growth factor-beta induces vascular endothelial growth factor-C expression leading to lymphangiogenesis in rat unilateral ureteral obstruction.” Kidney Int 81(9): 865-879.
65. Tanzer, A. and P. F. Stadler (2004). “Molecular evolution of a microRNA cluster.” J Mol Biol 339(2): 327-335.
66. Toffanin, S., D. Sia, et al. (2012). “microRNAs: new ways to block tumor angiogenesis?” Journal of Hepatology 57(3): 490-491.
67. Tsoyi, K., H. J. Jang, et al. (2010). “PTEN differentially regulates expressions of ICAM-1 and VCAM-1 through PI3K/Akt/GSK-3beta/GATA-6 signaling pathways in TNF-alpha-activated human endothelial cells.” Atherosclerosis 213(1): 115-121.
68. Urbich, C., A. Kuehbacher, et al. (2008). “Role of microRNAs in vascular diseases, inflammation, and angiogenesis.” Cardiovascular Research 79(4): 581-588.
69. Vass, D. G., B. Shrestha, et al. (2012). “Inflammatory lymphangiogenesis in a rat transplant model of interstitial fibrosis and tubular atrophy.” Transpl Int 25(7): 792-800.
70. Vigl, B., D. Aebischer, et al. (2011). “Tissue inflammation modulates gene expression of lymphatic endothelial cells and dendritic cell migration in a stimulus-dependent manner.” Blood 118(1): 205-215.
71. von der Weid, P. Y. and M. Muthuchamy (2010). “Regulatory mechanisms in lymphatic vessel contraction under normal and inflammatory conditions.” Pathophysiology: the official journal of the International Society for Pathophysiology/ISP 17(4): 263-276.
72. Watari, K., S. Nakao, et al. (2008). “Role of macrophages in inflammatory lymphangiogenesis: Enhanced production of vascular endothelial growth factor C and D through NF-kappaB activation.” Biochemical and Biophysical Research Communications 377(3): 826-831.
73. Wigle, J. T., N. Harvey, et al. (2002). “An essential role for Prox1 in the induction of the lymphatic endothelial cell phenotype.” The EMBO Journal 21(7): 1505-1513.
74. Wu, F., Z. Yang, et al. (2009). “Role of specific microRNAs for endothelial function and angiogenesis.” Biochemical and Biophysical Research Communications 386(4): 549-553.
75. Young, M. R., A. N. Santhanam, et al. (2010). “Have tumor suppressor PDCD4 and its counteragent oncogenic miR-21 gone rogue?” Mol Interv 10(2): 76-79.
76. Zgraggen, S., A. M. Ochsenbein, et al. (2013). “An important role of blood and lymphatic vessels in inflammation and allergy.” J Allergy (Cairo) 2013: 672381.
77. Zhuang, G., X. Wu, et al. (2012). “Tumour-secreted miR-9 promotes endothelial cell migration and angiogenesis by activating the JAK-STAT pathway.” The EMBO Journal 31(17): 3513-3523.

We claim:

1. A method of treating an inflammation mediated lymphatic disease in a mammal, the method comprising:
(a) detecting the level of expression of one or more miRNAs belonging to a profile of differentially expressed miRNAs in a lymphatic cell under a proinflammatory stimulus in:
A) a lymphatic cell obtained from the mammal, and
B) a control cell,
wherein a differential expression of the one or more miRNAs in the lymphatic cell obtained from the mammal as compared to the control cell is indicative of the presence of the inflammation mediated lymphatic disease in the mammal; and
(b) administering an effective amount of a therapeutic agent to the mammal to treat the inflammation mediated lymphatic disease.

2. The method according to claim 1, the one or more miRNAs belonging to a profile of differentially expressed miRNAs in a lymphatic cell under a proinflammatory stimulus are selected from miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p & miR-19b, miR-101, miR-144, miR-20a, miR448, miR-760-5p, miR-136, miR-141, miR-291a-3p, miR-327, miR-495, miR-136, miR-144, miR-145, miR-205, or a combination thereof.

3. A microarray chip corresponding to one or more of differentially expressed miRNAs in a lymphatic vessel cell under a proinflammatory stimulus, the microarray chip consisting essentially of oligonucleotides corresponding to one or more of:
a) miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p & miR-19b, miR-101, miR-144, miR-20a, miR448, miR-760-5p, miR-136, miR-141, miR-291a-3p, miR-327, miR-495, miR-136, miR-144, miR-145, and miR-205,
b) miR-181, miR-221, miR-222, miR-93, miR-200c, miR-17-5p, miR-203, miR-20a, miR-20b-5p, miR-21, miR-325-3p, miR-9, miR-27a, miR-322, miR-878, miR-19a, miR-497, miR-34c, miR-384-5p, and miR-19b, or
c) miR-101, miR-144, miR-20a, miR448, miR-760-5p, miR-136, miR-141, miR-291a-3p, miR-327, miR-495, miR-136, miR-144, miR-145 & miR-205.

4. The microarray chip according to claim 3, said microarray chip consisting essentially of oligonucleotides corresponding to:
a) miR-9 and miR-21,
b) miR-20a, miR-20b-5p, miR-21, miR-9, miR-145, miR-27a, miR-17-5p, miR-322, and miR-19b,
c) miR-141, miR-200c, miR-136, miR-21, and miR-9,
d) miR-34a, miR-34c, or
e) miR-203, miR-141, and miR-17-5p.

5. A method of identifying an agent as an activator or inhibitor of a miRNA, wherein the miRNA belongs to a profile of miRNAs differentially expressed in a lymphatic vessel cell under a proinflammatory stimulus, the method comprising:
a) culturing a first lymphatic vessel cell in the presence of the miRNA and in the absence of the agent,
b) culturing a second lymphatic vessel cell in the presence of the miRNA and in the presence of the agent,
c) determining the expression and/or activity of a target gene in the first and the second lymphatic cell,
d) comparing the expression and/or activity of the target gene in the first and the second lymphatic cell, and
e) identifying the agent as the inhibitor or the activator of the miRNA,
wherein the inhibitor of the miRNA negates the effect of the miRNA on the expression and/or activity of the target gene and the activator of the miRNA enhances the effect of the miRNA on the expression and/or activity of target gene.


SEQUENCE LISTING (150 sequences)

SEQ ID NO: 1 - DNA, 86 bp, Homo sapiens
  misc_feature: Pre-miRNA; hsa-miR-144-5p
uggggcccuggcugggauaucaucauauacuguaaguuugcgaugagacacuacaguauagaugauguacuaguccgggcaccccc

SEQ ID NO: 2 - DNA, 86 bp, Homo sapiens; identical to SEQ ID NO: 1
  misc_feature: Pre-miRNA; hsa-miR-144-3p

SEQ ID NO: 3 - DNA, 71 bp, Homo sapiens
  misc_feature: Pre-miRNA; hsa-miR-20a-5p
guagcacuaaagugcuuauagugcagguaguguuuaguuaucuacugcauuaugagcacuuaaaguacugc

SEQ ID NO: 4 - DNA, 71 bp, Homo sapiens; identical to SEQ ID NO: 3
  misc_feature: Pre-miRNA; hsa-miR-20a-3p

SEQ ID NO: 5 - DNA, 111 bp, Homo sapiens
  misc_feature: Pre-miRNA; hsa-miR-448
gccgggagguugaacauccugcauagugcugccaggaaaucccuauuucauauaagagggggcuggcugguugcauauguaggaugucccaucucccagcccacuucguca

SEQ ID NO: 6 - DNA, 68 bp, Homo sapiens
  misc_feature: Pre-miRNA; hsa-miR-200c-5p
cccucgucuuacccagcaguguuugggugcgguugggagucucuaauacugccggguaaugauggagg

SEQ ID NO: 7 - DNA, 68 bp, Homo sapiens; identical to SEQ ID NO: 6
  misc_feature: Pre-miRNA; hsa-miR-200c-3p

SEQ ID NO: 8 - DNA, 80 bp, Homo sapiens
  misc_feature: Pre-miRNA; hsa-miR-760
ggcgcgucgccccccucaguccaccagagcccggauaccucagaaauucggcucugggucuguggggagcgaaaugcaac

SEQ ID NO: 9 - DNA, 82 bp, Homo sapiens
  misc_feature: Pre-miRNA; hsa-miR-136-5p
ugagcccucggaggacuccauuuguuuugaugauggauucuuaugcuccaucaucgucucaaaugagucuucagaggguucu

SEQ ID NO: 10 - DNA, 82 bp, Homo sapiens; identical to SEQ ID NO: 9
  misc_feature: Pre-miRNA; hsa-miR-136-3p

SEQ ID NO: 11 - DNA, 95 bp, Homo sapiens
  misc_feature: Pre-miRNA; hsa-miR-141-5p
cggccggcccuggguccaucuuccaguacaguguuggauggucuaauugugaagcuccuaacacugucugguaaagauggcucccggguggguuc

SEQ ID NO: 12 - DNA, 95 bp, Homo sapiens; identical to SEQ ID NO: 11
  misc_feature: Pre-miRNA; hsa-miR-141-3p

SEQ ID NO: 13 - DNA, 84 bp, Homo sapiens
  misc_feature: Pre-miRNA; hsa-miR-17-5p
gucagaauaaugucaaagugcuuacagugcagguagugauaugugcaucuacugcagugaaggcacuuguagcauuauggugac

SEQ ID NO: 14 - DNA, 84 bp, Homo sapiens; identical to SEQ ID NO: 13
  misc_feature: Pre-miRNA; hsa-miR-17-3p

SEQ ID NO: 15 - DNA, 110 bp, Homo sapiens
  misc_feature: Pre-miRNA; hsa-miR-203a
guguuggggacucgcgcgcuggguccagugguucuuaacaguucaacaguucuguagcgcaauugugaaauguuuaggaccacuagacccggcgggcgcggcgacagcga

SEQ ID NO: 16 - DNA, 86 bp, Homo sapiens
  misc_feature: Pre-miRNA; hsa-miR-203b-5p
gcgcccgccgggucuagugguccuaaacauuucacaauugcgcuacagaacuguugaacuguuaagaaccacuggacccagcgcgc

SEQ ID NO: 17 - DNA, 86 bp, Homo sapiens; identical to SEQ ID NO: 16
  misc_feature: Pre-miRNA; hsa-miR-203b-3p

SEQ ID NO: 18 - DNA, 69 bp, Homo sapiens
  misc_feature: Pre-miRNA; hsa-miR-20b-5p
aguaccaaagugcucauagugcagguaguuuuggcaugacucuacuguaguaugggcacuuccaguacu

SEQ ID NO: 19 - DNA, 69 bp, Homo sapiens; identical to SEQ ID NO: 18
  misc_feature: Pre-miRNA; hsa-miR-20b-3p

SEQ ID NO: 20 - DNA, 72 bp, Homo sapiens
  misc_feature: Pre-miRNA; hsa-miR-21-5p
ugucggguagcuuaucagacugauguugacuguugaaucucauggcaacaccagucgaugggcugucugaca

SEQ ID NO: 21 - DNA, 72 bp, Homo sapiens; identical to SEQ ID NO: 20
  misc_feature: Pre-miRNA; hsa-miR-21-3p

SEQ ID NO: 22 - DNA, 98 bp, Homo sapiens
  misc_feature: Pre-miRNA; hsa-miR-325-3p
auacagugcuugguuccuaguagguguccaguaaguguuugugacauaauuuguuuauugaggaccuccuaucaaucaagcacugugcuaggcucugg

SEQ ID NO: 23 - DNA, 89 bp, Homo sapiens
  misc_feature: Pre-miRNA; hsa-miR-9-1-5p
cgggguugguuguuaucuuugguuaucuagcuguaugagugguguggagucuucauaaagcuagauaaccgaaaguaaaaauaacccca

SEQ ID NO: 24 - DNA, 89 bp, Homo sapiens; identical to SEQ ID NO: 23
  misc_feature: Pre-miRNA; hsa-miR-9-1-3p

SEQ ID NO: 25 - DNA, 87 bp, Homo sapiens
  misc_feature: Pre-miRNA; hsa-miR-9-2-5p
ggaagcgaguuguuaucuuugguuaucuagcuguaugaguguauuggucuucauaaagcuagauaaccgaaaguaaaaacuccuuca

SEQ ID NO: 26 - DNA, 87 bp, Homo sapiens; identical to SEQ ID NO: 25
  misc_feature: Pre-miRNA; hsa-miR-9-2-3p

SEQ ID NO: 27 - DNA, 90 bp, Homo sapiens
  misc_feature: Pre-miRNA; hsa-miR-9-3-5p
ggaggcccguuucucucuuugguuaucuagcuguaugagugccacagagccgucauaaagcuagauaaccgaaaguagaaaugauucuca

SEQ ID NO: 28 - DNA, 90 bp, Homo sapiens; identical to SEQ ID NO: 27
  misc_feature: Pre-miRNA; hsa-miR-9-3-3p

SEQ ID NO: 29 - DNA, 78 bp, Homo sapiens
  misc_feature: Pre-miRNA; hsa-miR-27a-5p
cugaggagcagggcuuagcugcuugugagcaggguccacaccaagucguguucacaguggcuaaguuccgccccccag

SEQ ID NO: 30 - DNA, 78 bp, Homo sapiens; identical to SEQ ID NO: 29
  misc_feature: Pre-miRNA; hsa-miR-27a-3p

SEQ ID NO: 31 - DNA, 95 bp, Homo sapiens
  misc_feature: Pre-miRNA; rno-miR-322-5p
ccucgcugacuccgaagggaugcagcagcaauucauguuuuggaguauugccaagguucaaaacaugaagcgcugcaacaccccuucgugggaaa

SEQ ID NO: 32 - DNA, 95 bp, Homo sapiens; identical to SEQ ID NO: 31
  misc_feature: Pre-miRNA; rno-miR-322-3p

SEQ ID NO: 33 - DNA, 77 bp, Homo sapiens
  misc_feature: Pre-miRNA; rno-miR-878-5p
ugcagugcuuuaucuaguuggcugucagucacgugaaacucaagugcaugacaccauacuggguagaggagggcuca

SEQ ID NO: 34 - DNA, 82 bp, Homo sapiens
  misc_feature: Pre-miRNA; hsa-miR-19a-5p
gcaguccucuguuaguuuugcauaguugcacuacaagaagaauguaguugugcaaaucuaugcaaaacugaugguggccugc

SEQ ID NO: 35 - DNA, 82 bp, Homo sapiens; identical to SEQ ID NO: 34
  misc_feature: Pre-miRNA; hsa-miR-19a-3p

SEQ ID NO: 36 - DNA, 82 bp, Homo sapiens
  misc_feature: Pre-miRNA; rno-miR-291a-5p
ccgguguaguagccaucaaaguggaggcccucucuugggcccgagcuagaaagugcuuccacuuugugugccacugcauggg

SEQ ID NO: 37 - DNA, 82 bp, Homo sapiens; identical to SEQ ID NO: 36
  misc_feature: Pre-miRNA; rno-miR-291a-3p

SEQ ID NO: 38 - DNA, 94 bp, Homo sapiens
  misc_feature: Pre-miRNA; rno-miR-327
gucugaugcccucauccuugaggggcaugaggguagucaguagccugaugucccucuugauggcacuucggacauguuggaauggcuugugagg

SEQ ID NO: 39 - DNA, 82 bp, Homo sapiens
  misc_feature: Pre-miRNA; hsa-miR-495-5p
ugguaccugaaaagaaguugcccauguuauuuucgcuuuauaugugacgaaacaaacauggugcacuucuuuuucgguauca

SEQ ID NO: 40 - DNA, 82 bp, Homo sapiens; identical to SEQ ID NO: 39
  misc_feature: Pre-miRNA; hsa-miR-495-3p

SEQ ID NO: 41 - DNA, 88 bp, Homo sapiens
  misc_feature: Pre-miRNA; hsa-miR-145-5p
caccuuguccucacgguccaguuuucccaggaaucccuuagaugcuaagauggggauuccuggaaauacuguucuugaggucaugguu

SEQ ID NO: 42 - DNA, 88 bp, Homo sapiens; identical to SEQ ID NO: 41
  misc_feature: Pre-miRNA; hsa-miR-145-3p

SEQ ID NO: 43 - DNA, 110 bp, Homo sapiens
  misc_feature: Pre-miRNA; hsa-miR-205-5p
aaagauccucagacaauccaugugcuucucuuguccuucauuccaccggagucugucucauacccaaccagauuucaguggagugaaguucaggaggcauggagcugaca

SEQ ID NO: 44 - DNA, 110 bp, Homo sapiens; identical to SEQ ID NO: 43
  misc_feature: Pre-miRNA; hsa-miR-205-3p

SEQ ID NO: 45 - DNA, 110 bp, Homo sapiens
  misc_feature: Pre-miRNA; hsa-miR-34a-5p
ggccagcugugaguguuucuuuggcagugucuuagcugguuguugugagcaauaguaaggaagcaaucagcaaguauacugcccuagaagugcugcacguuguggggccc

SEQ ID NO: 46 - DNA, 110 bp, Homo sapiens; identical to SEQ ID NO: 45
  misc_feature: Pre-miRNA; hsa-miR-34a-3p

SEQ ID NO: 47 - DNA, 112 bp, Homo sapiens
  misc_feature: Pre-miRNA; hsa-miR-497-5p
ccaccccgguccugcucccgccccagcagcacacugugguuuguacggcacuguggccacguccaaaccacacugugguguuagagcgagggugggggaggcaccgccgagg

SEQ ID NO: 48 - DNA, 112 bp, Homo sapiens; identical to SEQ ID NO: 47
  misc_feature: Pre-miRNA; hsa-miR-497-3p

SEQ ID NO: 49 - DNA, 77 bp, Homo sapiens
  misc_feature: Pre-miRNA; hsa-miR-34c-5p
agucuaguuacuaggcaguguaguuagcugauugcuaauaguaccaaucacuaaccacacggccagguaaaaagauu

SEQ ID NO: 50 - DNA, 77 bp, Homo sapiens; identical to SEQ ID NO: 49
  misc_feature: Pre-miRNA; hsa-miR-34c-3p

SEQ ID NO: 51 - DNA, 88 bp, Homo sapiens
  misc_feature: Pre-miRNA; hsa-miR-384-5p
uguuaaaucaggaauuuuaaacaauuccuagacaauauguauaauguucauaagucauuccuagaaauuguucauaaugccuguaaca

SEQ ID NO: 52 - DNA, 87 bp, Homo sapiens
  misc_feature: Pre-miRNA; hsa-miR-19b-1-5p
cacuguucuaugguuaguuuugcagguuugcauccagcugugugauauucugcugugcaaauccaugcaaaacugacugugguagug

SEQ ID NO: 53 - DNA, 87 bp, Homo sapiens; identical to SEQ ID NO: 52
  misc_feature: Pre-miRNA; hsa-miR-19b-1-3p

SEQ ID NO: 54 - DNA, 96 bp, Homo sapiens
  misc_feature: Pre-miRNA; hsa-miR-19b-1-5p
acauugcuacuuacaauuaguuuugcagguuugcauuucagcguauauauguauauguggcugugcaaauccaugcaaaacugauugugauaaugu

SEQ ID NO: 55 - DNA, 96 bp, Homo sapiens; identical to SEQ ID NO: 54
  misc_feature: Pre-miRNA; hsa-miR-19b-1-3p

SEQ ID NO: 56 - DNA, 110 bp, Homo sapiens
  misc_feature: Pre-miRNA; hsa-miR-181-a-1-5p
ugaguuuugagguugcuucagugaacauucaacgcugucggugaguuuggaauuaaaaucaaaaccaucgaccguugauuguacccuauggcuaaccaucaucuacucca

SEQ ID NO: 57 - DNA, 110 bp, Homo sapiens; identical to SEQ ID NO: 56
  misc_feature: Pre-miRNA; hsa-miR-181-a-1-3p

SEQ ID NO: 58 - DNA, 110 bp, Homo sapiens
  misc_feature: Pre-miRNA; hsa-miR-181-a-2-5p
agaagggcuaucaggccagccuucagaggacuccaaggaacauucaacgcugucggugaguuugggauuugaaaaaaccacugaccguugacuguaccuugggguccuua

SEQ ID NO: 59 - DNA, 110 bp, Homo sapiens; identical to SEQ ID NO: 58
  misc_feature: Pre-miRNA; hsa- miR-181-a-2-3p

SEQ ID NO: 60 - DNA, 110 bp, Homo sapiens
  misc_feature: Pre-miRNA; hsa- miR-181-b-1-5p
ccugugcagagauuauuuuuuaaaaggucacaaucaacauucauugcugucgguggguugaacuguguggacaagcucacugaacaaugaaugcaacuguggccccgcuu

SEQ ID NO: 61 - DNA, 110 bp, Homo sapiens; identical to SEQ ID NO: 60
  misc_feature: Pre-miRNA; hsa- miR-181-b-1-3p

SEQ ID NO: 62 - DNA, 89 bp, Homo sapiens
  misc_feature: Pre-miRNA; hsa- miR-181-b-2-5p
cugauggcugcacucaacauucauugcugucgguggguuugagucugaaucaacucacugaucaaugaaugcaaacugcggaccaaaca

SEQ ID NO: 63 - DNA, 110 bp, Homo sapiens
  misc_feature: Pre-miRNA; hsa- miR-181-c-5p
cggaaaauuugccaaggguuugggggaacauucaaccugucggugaguuugggcagcucaggcaaaccaucgaccguugaguggacccugaggccuggaauugccauccu

SEQ ID NO: 64 - DNA, 110 bp, Homo sapiens; identical to SEQ ID NO: 63
  misc_feature: Pre-miRNA; hsa- miR-181-c-3p

SEQ ID NO: 65 - DNA, 137 bp, Homo sapiens
  misc_feature: Pre-miRNA; hsa- miR-181-d-5p
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 66 - DNA, 137 bp, Homo sapiens; identical to SEQ ID NO: 65
  misc_feature: Pre-miRNA; hsa- miR-181-d-3p

SEQ ID NO: 67 - DNA, 110 bp, Homo sapiens
  misc_feature: Pre-miRNA; hsa- miR-221-5p
ugaacauccaggucuggggcaugaaccuggcauacaauguagauuucuguguucguuaggcaacagcuacauugucugcuggguuucaggcuaccuggaaacauguucuc

SEQ ID NO: 68 - DNA, 110 bp, Homo sapiens; identical to SEQ ID NO: 67
  misc_feature: Pre-miRNA; hsa- miR-221-3p

SEQ ID NO: 69 - DNA, 110 bp, Homo sapiens
  misc_feature: Pre-miRNA; hsa- miR-221-5p
gcugcuggaagguguagguacccucaauggcucaguagccaguguagauccugucuuucguaaucagcagcuacaucuggcuacugggucucugauggcaucuucuagcu

SEQ ID NO: 70 - DNA, 110 bp, Homo sapiens; identical to SEQ ID NO: 69
  misc_feature: Pre-miRNA; hsa- miR-221-3p

SEQ ID NO: 71 - DNA, 80 bp, Homo sapiens
  misc_feature: Pre-miRNA; hsa- miR-93-5p
cugggggcuccaaagugcuguucgugcagguagugugauuacccaaccuacugcugagcuagcacuucccgagcccccgg

SEQ ID NO: 72 - DNA, 80 bp, Homo sapiens; identical to SEQ ID NO: 71
  misc_feature: Pre-miRNA; hsa- miR-93-3p

SEQ ID NO: 73 - DNA, 75 bp, Homo sapiens
  misc_feature: Pre-miRNA; hsa- miR-101-1-5p
ugcccuggcucaguuaucacagugcugaugcugucuauucuaaagguacaguacugugauaacugaaggauggca

SEQ ID NO: 74 - DNA, 75 bp, Homo sapiens; identical to SEQ ID NO: 73
  misc_feature: Pre-miRNA; hsa- miR-101-1-3p

SEQ ID NO: 75 - DNA, 79 bp, Homo sapiens
  misc_feature: Pre-miRNA; hsa- miR-101-2-3p
acuguccuuuuucgguuaucaugguaccgaugcuguauaucugaaagguacaguacugugauaacugaagaaugguggu

SEQ ID NO: 76 - DNA, 22 bp, Homo sapiens
  misc_feature: Mature miRNA; hsa-miR-144-5p
ggauaucaucauauacuguaag

SEQ ID NO: 77 - DNA, 20 bp, Homo sapiens
  misc_feature: Mature miRNA; hsa-miR-144-3p
uacaguauagaugauguacu

SEQ ID NO: 78 - DNA, 23 bp, Homo sapiens
  misc_feature: Mature miRNA; hsa-miR-20a-5p
uaaagugcuuauagugcagguag

SEQ ID NO: 79 - DNA, 22 bp, Homo sapiens
  misc_feature: Mature miRNA; hsa-miR-20a-3p
acugcauuaugagcacuuaaag

SEQ ID NO: 80 - DNA, 22 bp, Homo sapiens
  misc_feature: Mature miRNA; hsa-miR-448
uugcauauguaggaugucccau

SEQ ID NO: 81 - DNA, 22 bp, Homo sapiens
  misc_feature: Mature miRNA; hsa-miR-200c-5p
cgucuuacccagcaguguuugg

SEQ ID NO: 82 - DNA, 23 bp, Homo sapiens
  misc_feature: Mature miRNA; hsa-miR-200c-3p
uaauacugccggguaaugaugga

SEQ ID NO: 83 - DNA, 20 bp, Homo sapiens
  misc_feature: Mature miRNA; hsa-miR-760
cggcucugggucugugggga

SEQ ID NO: 84 - DNA, 23 bp, Homo sapiens
  misc_feature: Mature miRNA; hsa-miR-136-5p
acuccauuuguuuugaugaugga

SEQ ID NO: 85 - DNA, 22 bp, Homo sapiens
  misc_feature: Mature miRNA; hsa-miR-136-3p
caucaucgucucaaaugagucu

SEQ ID NO: 86 - DNA, 22 bp, Homo sapiens
  misc_feature: Mature miRNA; hsa-miR-141-5p
caucuuccaguacaguguugga

SEQ ID NO: 87 - DNA, 22 bp, Homo sapiens
  misc_feature: Mature miRNA; hsa-miR-141-3p
uaacacugucugguaaagaugg

SEQ ID NO: 88 - DNA, 23 bp, Homo sapiens
  misc_feature: Mature miRNA; hsa-miR-17-5p
caaagugcuuacagugcagguag

SEQ ID NO: 89 - DNA, 22 bp, Homo sapiens
  misc_feature: Mature miRNA; hsa-miR-17-3p
acugcagugaaggcacuuguag

SEQ ID NO: 90 - DNA, 22 bp, Homo sapiens
  misc_feature: Mature miRNA; hsa-miR-203a
gugaaauguuuaggaccacuag

SEQ ID NO: 91 - DNA, 22 bp, Homo sapiens
  misc_feature: Mature miRNA; hsa-miR-203b-5p
uagugguccuaaacauuucaca

SEQ ID NO: 92 - DNA, 23 bp, Homo sapiens
  misc_feature: Mature miRNA; hsa-miR-203b-3p
uugaacuguuaagaaccacugga

SEQ ID NO: 93 - DNA, 23 bp, Homo sapiens
  misc_feature: Mature miRNA; hsa-miR-20b-5p
caaagugcucauagugcagguag

SEQ ID NO: 94 - DNA, 22 bp, Homo sapiens
  misc_feature: Mature miRNA; hsa-miR-20b-3p
acuguaguaugggcacuuccag

SEQ ID NO: 95 - DNA, 22 bp, Homo sapiens
  misc_feature: Mature miRNA; hsa-miR-21-5p
uagcuuaucagacugauguuga

SEQ ID NO: 96 - DNA, 21 bp, Homo sapiens
  misc_feature: Mature miRNA; hsa-miR-21-3p
caacaccagucgaugggcugu

SEQ ID NO: 97 - DNA, 23 bp, Homo sapiens
  misc_feature: Mature miRNA; hsa-miR-325-3p
ccuaguagguguccaguaagugu

SEQ ID NO: 98 - DNA, 23 bp, Homo sapiens
  misc_feature: Mature miRNA; hsa-miR-9-1-5p
ucuuugguuaucuagcuguauga

SEQ ID NO: 99 - DNA, 22 bp, Homo sapiens
  misc_feature: Mature miRNA; hsa-miR-9-1-3p
auaaagcuagauaaccgaaagu

SEQ ID NO: 100 - DNA, 23 bp, Homo sapiens
  misc_feature: Mature miRNA; hsa-miR-9-2-5p
ucuuugguuaucuagcuguauga

SEQ ID NO: 101 - DNA, 22 bp, Homo sapiens
  misc_feature: Mature miRNA; hsa-miR-9-2-3p
auaaagcuagauaaccgaaagu

SEQ ID NO: 102 - DNA, 23 bp, Homo sapiens
  misc_feature: Mature miRNA; hsa-miR-9-3-5p
ucuuugguuaucuagcuguauga

SEQ ID NO: 103 - DNA, 22 bp, Homo sapiens
  misc_feature: Mature miRNA; hsa-miR-9-3-3p
auaaagcuagauaaccgaaagu

SEQ ID NO: 104 - DNA, 22 bp, Homo sapiens
  misc_feature: Mature miRNA; hsa-miR-27a-5p
agggcuuagcugcuugugagca

SEQ ID NO: 105 - DNA, 21 bp, Homo sapiens
  misc_feature: Mature miRNA; hsa-miR-27a-3p
uucacaguggcuaaguuccgc

SEQ ID NO: 106 - DNA, 22 bp, Homo sapiens
  misc_feature: Mature miRNA; rno-miR-322-5p
cagcagcaauucauguuuugga

SEQ ID NO: 107 - DNA, 20 bp, Homo sapiens
  misc_feature: Mature miRNA; rno-miR-322-5p
aaacaugaagcgcugcaaca

SEQ ID NO: 108 - DNA, 22 bp, Homo sapiens
  misc_feature: Mature miRNA; rno-miR-878-5p
gcaugacaccauacuggguaga

SEQ ID NO: 109 - DNA, 22 bp, Homo sapiens
  misc_feature: Mature miRNA; hsa-miR-19a-5p
aguuuugcauaguugcacuaca

SEQ ID NO: 110 - DNA, 23 bp, Homo sapiens
  misc_feature: Mature miRNA; hsa-miR-19a-3p
ugugcaaaucuaugcaaaacuga

SEQ ID NO: 111 - DNA, 22 bp, Homo sapiens
  misc_feature: Mature miRNA; rno-miR-291a-5p
caucaaaguggaggcccucucu

SEQ ID NO: 112 - DNA, 23 bp, Homo sapiens
  misc_feature: Mature miRNA; rno-miR-291a-3p
aaagugcuuccacuuugugugcc

SEQ ID NO: 113 - DNA, 19 bp, Homo sapiens
  misc_feature: Mature miRNA; rno-miR-327
ccuugaggggcaugagggu

SEQ ID NO: 114 - DNA, 22 bp, Homo sapiens
  misc_feature: Mature miRNA; hsa-miR-495-5p
gaaguugcccauguuauuuucg

SEQ ID NO: 115 - DNA, 22 bp, Homo sapiens
  misc_feature: Mature miRNA; hsa-miR-495-3p
aaacaaacauggugcacuucuu

SEQ ID NO: 116 - DNA, 23 bp, Homo sapiens
  misc_feature: Mature miRNA; hsa-miR-145-5p
guccaguuuucccaggaaucccu

SEQ ID NO: 117 - DNA, 22 bp, Homo sapiens
  misc_feature: Mature miRNA; hsa-miR-145-3p
ggauuccuggaaauacuguucu

SEQ ID NO: 118 - DNA, 22 bp, Homo sapiens
  misc_feature: Mature miRNA; hsa-miR-205-5p
uccuucauuccaccggagucug

SEQ ID NO: 119 - DNA, 21 bp, Homo sapiens
  misc_feature: Mature miRNA; hsa-miR-205-3p
gauuucaguggagugaaguuc

SEQ ID NO: 120 - DNA, 22 bp, Homo sapiens
  misc_feature: Mature miRNA; hsa-miR-34a-5p
uggcagugucuuagcugguugu

SEQ ID NO: 121 - DNA, 22 bp, Homo sapiens
  misc_feature: Mature miRNA; hsa-miR-34a-3p
caaucagcaaguauacugcccu

SEQ ID NO: 122 - DNA, 21 bp, Homo sapiens
  misc_feature: Mature miRNA; hsa-miR-497-5p
cagcagcacacugugguuugu

SEQ ID NO: 123 - DNA, 22 bp, Homo sapiens
  misc_feature: Mature miRNA; hsa-miR-497-3p
caaaccacacugugguguuaga

SEQ ID NO: 124 - DNA, 23 bp, Homo sapiens
  misc_feature: Mature miRNA; hsa-miR-34c-5p
aggcaguguaguuagcugauugc

SEQ ID NO: 125 - DNA, 22 bp, Homo sapiens
  misc_feature: Mature miRNA; hsa-miR-34c-3p
aaucacuaaccacacggccagg

SEQ ID NO: 126 - DNA, 20 bp, Homo sapiens
  misc_feature: Mature miRNA; hsa-miR-384-5p
auuccuagaaauuguucaua

SEQ ID NO: 127 - DNA, 23 bp, Homo sapiens
  misc_feature: Mature miRNA; hsa-miR-19b-1-5p
aguuuugcagguuugcauccagc

SEQ ID NO: 128 - DNA, 23 bp, Homo sapiens
  misc_feature: Mature miRNA; hsa-miR-19b-1-3p
ugugcaaauccaugcaaaacuga

SEQ ID NO: 129 - DNA, 22 bp, Homo sapiens
  misc_feature: Mature miRNA; hsa-miR-19b-1-5p
aguuuugcagguuugcauuuca

SEQ ID NO: 130 - DNA, 23 bp, Homo sapiens
  misc_feature: Mature miRNA; hsa-miR-19b-1-3p
ugugcaaauccaugcaaaacuga

SEQ ID NO: 131 - DNA, 23 bp, Homo sapiens
  misc_feature: Mature miRNA; hsa-miR-181-a-1-5p
aacauucaacgcugucggugagu

SEQ ID NO: 132 - DNA, 22 bp, Homo sapiens
  misc_feature: Mature miRNA; hsa-miR-181-a-1-3p
accaucgaccguugauuguacc

SEQ ID NO: 133 - DNA, 23 bp, Homo sapiens
  misc_feature: Mature miRNA; hsa-miR-181-a-2-5p
aacauucaacgcugucggugagu

SEQ ID NO: 134 - DNA, 22 bp, Homo sapiens
  misc_feature: Mature miRNA; hsa- miR-181-a-2-3p
accacugaccguugacuguacc

SEQ ID NO: 135 - DNA, 23 bp, Homo sapiens
  misc_feature: Mature miRNA; hsa- miR-181-b-1-5p
aacauucauugcugucggugggu

SEQ ID NO: 136 - DNA, 21 bp, Homo sapiens
  misc_feature: Mature miRNA; hsa- miR-181-b-1-3p
cucacugaacaaugaaugcaa

SEQ ID NO: 137 - DNA, 23 bp, Homo sapiens
  misc_feature: Mature miRNA; hsa- miR-181-b-2-5p
aacauucauugcugucggugggu

SEQ ID NO: 138 - DNA, 22 bp, Homo sapiens
  misc_feature: Mature miRNA; hsa- miR-181-c-5p
aacauucaaccugucggugagu

SEQ ID NO: 139 - DNA, 22 bp, Homo sapiens
  misc_feature: Mature miRNA; hsa- miR-181-c-3p
aaccaucgaccguugaguggac

SEQ ID NO: 140 - DNA, 23 bp, Homo sapiens
  misc_feature: Mature miRNA; hsa- miR-181-d-5p
aacauucauuguugucggugggu

SEQ ID NO: 141 - DNA, 21 bp, Homo sapiens
  misc_feature: Mature miRNA; hsa- miR-181-d-3p
ccaccgggggaugaaugucac

SEQ ID NO: 142 - DNA, 22 bp, Homo sapiens
  misc_feature: Mature miRNA; hsa- miR-221-5p
accuggcauacaauguagauuu

SEQ ID NO: 143 - DNA, 23 bp, Homo sapiens
  misc_feature: Mature miRNA; hsa- miR-221-3p
agcuacauugucugcuggguuuc

SEQ ID NO: 144 - DNA, 22 bp, Homo sapiens
  misc_feature: Mature miRNA; hsa- miR-221-5p
cucaguagccaguguagauccu

SEQ ID NO: 145 - DNA, 21 bp, Homo sapiens
  misc_feature: Mature miRNA; hsa- miR-221-3p
agcuacaucuggcuacugggu

SEQ ID NO: 146 - DNA, 23 bp, Homo sapiens
  misc_feature: Mature miRNA; hsa- miR-93-5p
caaagugcuguucgugcagguag

SEQ ID NO: 147 - DNA, 22 bp, Homo sapiens
  misc_feature: Mature miRNA; hsa- miR-93-3p
acugcugagcuagcacuucccg

SEQ ID NO: 148 - DNA, 22 bp, Homo sapiens
  misc_feature: Mature miRNA; hsa- miR-101-1-5p
caguuaucacagugcugaugcu

SEQ ID NO: 149 - DNA, 21 bp, Homo sapiens
  misc_feature: Mature miRNA; hsa- miR-101-1-3p
uacaguacugugauaacugaa

SEQ ID NO: 150 - DNA, 21 bp, Homo sapiens
  misc_feature: Mature miRNA; hsa- miR-101-2-3p
uacaguacugugauaacugaa